(19) United States
(12) Patent Application Publication
(10) Pub. No.: US 2022/0000932 A1
(43) Pub. Date: Jan. 6, 2022

(54) USE OF EXTRACELLULAR VESICLES IN COMBINATION WITH TISSUE PLASMINOGEN ACTIVATOR AND/OR THROMBECTOMY TO TREAT STROKE

(71) Applicant: Henry Ford Health System, Detroit, MI (US)

(72) Inventors: Zhenggang Zhang, Troy, MI (US); Li Zhang, Troy, MI (US); Michael Chopp, Southfield, MI (US); Benjamin Buller, Bloomfield Hills, MI (US)

(73) Assignee: Henry Ford Health System, Detroit, MI (US)

(21) Appl. No.: 17/280,794

(22) PCT Filed: Sep. 27, 2019
(86) PCT No.: PCT/US2019/053452

§ 371 Date: Mar. 26, 2021

Related U.S. Application Data
(60) Provisional application No. 62/738,465, filed on Sep. 28, 2018.

(51) Int. Cl. A61K 35/28; A61K 35/30; A61K 38/49; A61K 31/7125

(52) U.S. Cl. CPC A61K 35/28; A61K 31/7125; A61K 38/49; A61K 35/30

(57) ABSTRACT

Some embodiments comprise a method and kit for the treatment and prevention of stroke by administering a therapeutically effective combination of mammalian exosomes and/or microvesicles, collectively referred to as extracellular vesicles, and Tissue Plasminogen Activator (tPA), and/or a thrombectomy procedure, to a subject in need thereof. Some embodiments comprise a method and kit for the treatment and prevention of cerebrovascular injury caused by a stroke by administering a therapeutically effective combination of mammalian exosomes, Tissue Plasminogen Activator (tPA), and/or a thrombectomy procedure, to a subject in need thereof. Some embodiments also comprise the administration a therapeutically effective amount of a combination comprising mammalian exosomes and Tissue Plasminogen Activator (tPA) to a subject in need thereof; the mammalian exosomes containing one or more microRNAs selected from miR-19a, miR-21, and miR-146a.

### RELATED APPLICATIONS

This application claims priority to, and the benefit of, U.S. Provisional Application No. 62/738,465, filed Sep. 28, 2018, the contents of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Sep. 26, 2019, is named “NEUX-009_001WO_SeqList.txt” and is 3.84 KB in size.

### TECHNICAL FIELD

Without limitation, some embodiments comprise methods, systems, and compositions relating to the treatment of stroke with a therapeutically effective combination of mammalian extracellular vesicles (i.e. exosomes and/or microvesicles) and tissue plasminogen activator (tPA), and/or thrombectomy.

### BACKGROUND

Stroke is the fifth leading cause of death and the first cause of disability worldwide. Large cerebral vessel occlusion which constitutes approximately 25% of ischemic strokes is the most disabling and life-threatening form of ischemic stroke. Stroke is a prominent cause of mortality and long-term disability and is accompanied by unusually high social and medical costs. The major causes of death in stroke-related mortalities are a consequence of neurological damage and/or cardiovascular complications.

Acute ischemic stroke is the sudden blockage of adequate blood flow to a section of the brain, usually caused by thrombus or other emboli lodging or forming in one of the blood vessels supplying the brain. If this blockage is not quickly resolved, the ischemia may lead to permanent neurologic deficit or death. The timeframe for effective treatment of stroke in the United States is within 3 hours for intravenous (IV) thrombolytic therapy and within 24 hours for site-directed intra-arterial thrombolytic therapy or interventional recanalization of a blocked cerebral artery. Reperfusing the ischemic brain after this time period has no overall benefit to the patient, and may in fact cause harm due to the increased risk of intracranial hemorrhage from fibrinolytic use. Even within this time period, there is strong evidence that the shorter the time period between onset of symptoms and treatment, the better the results. Unfortunately, the ability to recognize symptoms, deliver patients to stroke treatment sites, and finally to treat these patients within this timeframe is rare. Despite treatment advances, stroke remains the third leading cause of death in the United States.

Endovascular treatment of acute stroke is comprised of either the intra-arterial administration of thrombolytic drugs such as tissue plasminogen activator (tPA), mechanical removal of the blockage, or a combination of the two. As mentioned above, these interventional treatments must occur within hours of the onset of symptoms. Both intra-arterial (IA) thrombolytic therapy and interventional thrombectomy involve accessing the blocked cerebral artery via endovascular techniques and devices.

Like IV thrombolytic therapy, IA thrombolytic therapy alone has the limitation in that it may take several hours of infusion to effectively dissolve the clot. Mechanical therapies have involved capturing and removing the clot, dissolving the clot, disrupting and suctioning the clot, and/or creating a flow channel through the clot. However, this is at the expense of an increase in the rate of symptomatic intracranial hemorrhage to 10%. To improve the rate of recanalization, expand the time window, and reduce the risk of symptomatic intracranial hemorrhage, mechanical thrombectomy was introduced, with initial approval of the Merci clot retriever, a corkscrew-like device, and then subsequently with approval of the Penumbra thromboaspiration system. Both devices are associated with a high rate of recanalization (total, partial, and complete). However, time to recanalization was on average 45 minutes, with a low rate of complete clot resolution, given that the majority of patients achieved only partial recanalization. More recently, retrievable stents have shown promise in reducing the time to recanalization, and they achieve a higher rate of complete clot resolution with improved feasibility. The retrievable stent can be opened within the clot to engage it within the stent struts, and subsequently it is retrieved by pulling it under flow arrest. The retrievable stents provide a new tool in the armamentarium of devices that can be used to achieve safe and timely clot removal. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Frank suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Standard intravenous thrombolysis with tissue plasminogen activator (tPA) for the treatment eligible patients results in only one third of patients experiencing early brain reperfusion. Thrombectomy performed within 6 hours of stroke onset is now also a standard of care for treatment of acute ischemic stroke with large vessel occlusion. Reperfusion of the ischemic lesion is closely associated with good clinical outcome. However, recanalization of the occluded large artery by thrombectomy only leads to 71% of patients achieving improved tissue reperfusion. In addition, due to unfavorably large ischemic lesion cores, many patients with large vessel occlusion are not eligible to receive tPA or endovascular therapy. Aging has been shown to potentiate secondary thrombosis and vascular damage in the ischemic brain after tPA treatment.

The inventors have demonstrated that tPA administered to aged rats 2 h after embolic middle cerebral artery occlusion (MCAO, an established animal model of stroke) does not increase mortality, but fails to reduce ischemic brain damage (cerebrovascular injury), and aggravates the neurovascular damage characterized by acute activation of vascular prothrombotic/proinflammatory signals. Thus, there is a compelling need to develop therapies to block ischemic core expansion, thereby to increase numbers of patients who would be eligible to receive tPA and thrombectomy, and importantly, to augment tissue reperfusion, and thereby achieve improved functional outcome. Various embodiments of the present disclosure address and resolve these pressing needs.

### SUMMARY OF THE INVENTION

The present disclosure provides a method for treating stroke in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the treatment of stroke, wherein the combination of mammalian exosomes and tPA is for administration to a subject in need thereof in a therapeutically effective amount. The present disclosure provides a combination comprising mammalian exosomes and tPA for the manufacture of a medicament for the treatment of stroke, wherein the combination of mammalian exosomes and tPA is for administration to a subject in need thereof in a therapeutically effective amount.

The present disclosure provides a method for reducing the expansion of an ischemic core after stroke in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the reduction of the expansion of an ischemic core after stroke in a subject, wherein the combination of mammalian exosomes and tPA is for administration to a subject in need thereof in a therapeutically effective amount. The present disclosure provides a combination comprising mammalian exosomes and tPA for the manufacture of a medicament for the reduction of the expansion of an ischemic core after stroke in a subject, wherein the combination of mammalian exosomes and tPA is for administration to a subject in need thereof in a therapeutically effective amount.

The present disclosure provides a method for treating or preventing cerebrovascular injury in a subject, the method comprising administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of cerebrovascular injury, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of cerebrovascular injury, wherein the combination of mammalian exosomes and tPA is for administration to a subject in need thereof in a therapeutically effective amount.

The present disclosure provides a method for treating or preventing cerebrovascular injury in a subject, the method comprising administering a therapeutically effective amount of mammalian exosomes to and performing a thrombectomy on a subject in need thereof. The present disclosure provides mammalian exosomes for use in the treatment or prevention of cerebrovascular injury in a subject, wherein the treatment or prevention further comprises performing a thrombectomy. The present disclosure provides mammalian exosomes for the manufacture of a medicament for the treatment or prevention of stroke, wherein the mammalian exosomes are for administration to a subject in need thereof in a therapeutically effective amount, and wherein the treatment further comprises performing a thrombectomy.

The present disclosure provides a method for treating stroke in a subject, the method comprising administering a therapeutically effective amount of mammalian exosomes to and performing a thrombectomy on a subject in need thereof. The present disclosure provides mammalian exosomes for use in the treatment of stroke in a subject, wherein the treatment further comprises performing a thrombectomy, and wherein the mammalian exosomes are for administration to the subject in a therapeutically effective amount. The present disclosure provides mammalian exosomes for the manufacture of a medicament for the treatment of stroke, wherein the mammalian exosomes are for administration to a subject in need thereof in a therapeutically effective amount, and wherein the treatment further comprises performing a thrombectomy, and wherein the mammalian exosomes are for administration to the subject in a therapeutically effective amount.

The present disclosure provides a method for treating or preventing secondary thrombosis in downstream brain microvessels in a subject, the method comprising: administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of secondary thrombosis in downstream brain microvessels, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of secondary thrombosis in downstream brain microvessels, wherein the combination of mammalian exosomes and tPA is for administration to a subject in need thereof in a therapeutically effective amount.

The present disclosure provides a method for treating or preventing blood brain barrier impairment in a subject, the method comprising: administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of blood brain barrier impairment, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of blood brain barrier impairment, wherein the combination of mammalian exosomes and tPA is for administration to a subject in need thereof in a therapeutically effective amount.

The present disclosure provides a method of treating or preventing cerebrovascular injury in a subject, the method comprising administering a therapeutically effective combination of mammalian exosomes and Tissue Plasminogen Activator (tPA) to and performing a thrombectomy on a subject in need thereof. The present disclosure provides a combination comprising mammalian exosomes and Tissue Plasminogen Activator (tPA) for use in the treatment or prevention of cerebrovascular injury in a subject, wherein the treatment or prevention further comprises performing a thrombectomy, and wherein the mammalian exosomes and tPA are for administration to the subject in a therapeutically effective amount. The present disclosure provides mammalian exosomes for use in the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides tissue Plasminogen Activator (tPA) for use in the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides a combination comprising mammalian exosomes and Tissue Plasminogen Activator (tPA) for use in the manufacture of a medicament for the treatment or prevention of cerebrovascular injury in a subject, wherein the treatment or prevention further comprises performing a thrombectomy, and wherein the mammalian exosomes and tPA are for administration to the subject in a therapeutically effective amount. The present disclosure provides mammalian exosomes for use in the manufacture of a medicament for the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides tissue Plasminogen Activator (tPA) for use in the manufacture of a medicament for the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy.

The present disclosure provides a method of treating stroke in a subject, the method comprising administering a therapeutically effective combination of mammalian exosomes and tPA to and performing a thrombectomy on a subject in need thereof. The present disclosure provides a combination comprising mammalian exosomes and tPA for use in the treatment of stroke in a subject, wherein the treatment further comprises performing a thrombectomy, and wherein the mammalian exosomes and tPA are for administration to the subject in a therapeutically effective amount. The present disclosure provides mammalian exosomes for use in the treatment of stroke, the treatment comprising administering a combination comprising mammalian exosomes and tPA, and wherein the treatment further comprises performing a thrombectomy. The present disclosure provides tPA for use in the treatment of stroke, the treatment comprising administering a combination comprising mammalian exosomes and tPA, and wherein the treatment further comprises performing a thrombectomy. The present disclosure provides a combination comprising mammalian exosomes and tPA for use in the manufacture of a medicament for the treatment of stroke in a subject, wherein the treatment further comprises performing a thrombectomy, and wherein the mammalian exosomes and tPA are for administration to the subject in a therapeutically effective amount. The present disclosure provides mammalian exosomes for use in the manufacture of a medicament for the treatment of, the treatment comprising administering a combination comprising mammalian exosomes and tPA, and wherein the treatment further comprises performing a thrombectomy. The present disclosure provides tPA for use in the manufacture of a medicament for the treatment of stroke, the treatment comprising administering a combination comprising mammalian exosomes and tPA, and wherein the treatment further comprises performing a thrombectomy.

The present disclosure provides a method for treating or preventing of blood brain barrier leakage in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the treatment or prevention of blood brain barrier leakage, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of blood brain barrier leakage, wherein the combination of mammalian exosomes and tPA is for administration to a subject in need thereof in a therapeutically effective amount.

The present disclosure provides a method for treating stroke in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the treatment of stroke, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment of stroke, wherein the combination of mammalian exosomes and at least one thrombolytic agent is for administration to a subject in need thereof in a therapeutically effective amount.

The present disclosure provides a method for reducing the expansion of an ischemic core after stroke in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the reduction of the expansion of an ischemic core after stroke in a subject, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the reduction of the expansion of an ischemic core after stroke in a subject, wherein the combination of mammalian exosomes and at least one thrombolytic agent is for administration to a subject in need thereof in a therapeutically effective amount.

The present disclosure provides a method for treating or preventing cerebrovascular injury in a subject, the method comprising administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of cerebrovascular injury, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of cerebrovascular injury, wherein the combination of mammalian exosomes and at least one thrombolytic agent is for administration to a subject in need thereof in a therapeutically effective amount.

The present disclosure provides a method for treating or preventing cerebrovascular injury in a subject, the method comprising administering a therapeutically effective amount of mammalian exosomes to and performing a thrombectomy on a subject in need thereof. The present disclosure provides mammalian exosomes for use in the treatment or prevention of cerebrovascular injury in a subject, wherein the treatment or prevention further comprises performing a thrombectomy. The present disclosure provides mammalian exosomes for the manufacture of a medicament for the treatment or prevention of stroke, wherein the mammalian exosomes are for administration to a subject in need thereof in a therapeutically effective amount, and wherein the treatment further comprises performing a thrombectomy.

The present disclosure provides a method for treating stroke in a subject, the method comprising administering a therapeutically effective amount of mammalian exosomes to and performing a thrombectomy on a subject in need thereof. The present disclosure provides mammalian exosomes for use in the treatment of stroke in a subject, wherein the treatment further comprises performing a thrombectomy, and wherein the mammalian exosomes are for administration to the subject in a therapeutically effective amount. The present disclosure provides mammalian exosomes for the manufacture of a medicament for the treatment of stroke, wherein the mammalian exosomes are for administration to a subject in need thereof in a therapeutically effective amount, and wherein the treatment further comprises performing a thrombectomy, and wherein the mammalian exosomes are for administration to the subject in a therapeutically effective amount.

The present disclosure provides a method for treating or preventing secondary thrombosis in downstream brain microvessels in a subject, the method comprising: administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of secondary thrombosis in downstream brain microvessels, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of secondary thrombosis in downstream brain microvessels, wherein the combination of mammalian exosomes and at least one thrombolytic agent is for administration to a subject in need thereof in a therapeutically effective amount.

The present disclosure provides a method for treating or preventing blood brain barrier impairment in a subject, the method comprising: administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of blood brain barrier impairment, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of blood brain barrier impairment, wherein the combination of mammalian exosomes and at least one thrombolytic agent is for administration to a subject in need thereof in a therapeutically effective amount.

The present disclosure provides a method of treating or preventing cerebrovascular injury in a subject, the method comprising administering a therapeutically effective combination of mammalian exosomes and at least one thrombolytic agent to and performing a thrombectomy on a subject in need thereof. The present disclosure provides a combination comprising mammalian exosomes and at least one thrombolytic agent for use in the treatment or prevention of cerebrovascular injury in a subject, wherein the treatment or prevention further comprises performing a thrombectomy, and wherein the mammalian exosomes and at least one thrombolytic agent are for administration to the subject in a therapeutically effective amount. The present disclosure provides mammalian exosomes for use in the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides at least one thrombolytic agent for use in the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides a combination comprising mammalian exosomes and at least one thrombolytic agent for use in the manufacture of a medicament for the treatment or prevention of cerebrovascular injury in a subject, wherein the treatment or prevention further comprises performing a thrombectomy, and wherein the mammalian exosomes and at least one thrombolytic agent are for administration to the subject in a therapeutically effective amount. The present disclosure provides mammalian exosomes for use in the manufacture of a medicament for the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides at least one thrombolytic agent for use in the manufacture of a medicament for the treatment or prevention of cerebrovascular injury, the treatment or prevention comprising administering a combination comprising mammalian exosomes and at least one thrombolytic agent, and wherein the treatment or prevention further comprises performing a thrombectomy.

The present disclosure provides a method of treating stroke in a subject, the method comprising administering a therapeutically effective combination of mammalian exosomes and at least one thrombolytic agent to and performing a thrombectomy on a subject in need thereof. The present disclosure provides a combination comprising mammalian exosomes and at least one thrombolytic agent for use in the treatment of stroke in a subject, wherein the treatment further comprises performing a thrombectomy, and wherein the mammalian exosomes and at least one thrombolytic agent are for administration to the subject in a therapeutically effective amount. The present disclosure provides mammalian exosomes for use in the treatment of stroke, the treatment comprising administering a combination comprising mammalian exosomes and at least one thrombolytic agent, and wherein the treatment further comprises performing a thrombectomy. The present disclosure provides at least one thrombolytic agent for use in the treatment of stroke, the treatment comprising administering a combination comprising mammalian exosomes and at least one thrombolytic agent, and wherein the treatment further comprises performing a thrombectomy. The present disclosure provides a combination comprising mammalian exosomes and at least one thrombolytic agent for use in the manufacture of a medicament for the treatment of stroke in a subject, wherein the treatment further comprises performing a thrombectomy, and wherein the mammalian exosomes and at least one thrombolytic agent are for administration to the subject in a therapeutically effective amount. The present disclosure provides mammalian exosomes for use in the manufacture of a medicament for the treatment of, the treatment comprising administering a combination comprising mammalian exosomes and at least one thrombolytic agent, and wherein the treatment further comprises performing a thrombectomy. The present disclosure provides at least one thrombolytic agent for use in the manufacture of a medicament for the treatment of stroke, the treatment comprising administering a combination comprising mammalian exosomes and at least one thrombolytic agent, and wherein the treatment further comprises performing a thrombectomy.

The present disclosure provides a method for treating or preventing of blood brain barrier leakage in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the treatment or prevention of blood brain barrier leakage, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of blood brain barrier leakage, wherein the combination of mammalian exosomes and at least one thrombolytic agent is for administration to a subject in need thereof in a therapeutically effective amount.

Any of the preceding methods can further comprise performing a thrombectomy on the subject.

In some aspects of the preceding methods, treating, preventing or treating or preventing can comprise any one of the following or any combination of the following: (a) increasing proteolysis of fibrin in a clot and/or thrombus, (b) increasing the rate and extent of vessel recanalization, (c) increasing microvascular reperfusion without increasing brain hemorrhage, (d) reducing leakage of the blood-brain-barrier, (e) attenuating infarct expansion, (f) reducing prothrombotic procoagulant vascular conditions, (g) reducing vascular and/or cerebral brain cell inflammation, (h) reducing prothrombotic procoagulant vascular conditions and vascular and subsequent cerebral brain cell inflammation, (i) extending the therapeutic window for tPA treatment, (j), reducing the size of a clot or thrombus, (k) reducing adhesion molecules, (l) reducing vascular inflammation, (m) reducing procoagulant and/or prothrombotic conditions, (n) reducing the expansion of an ischemic core, (o) reducing infarct volume, (p) improving neurological outcome, (q) enhancing tissue perfusion, (r) extending the therapeutic window for treatment with at least one thrombolytic agent.

The subject can be a subject that has suffered a stroke. A subject can be a human. A stroke can be an ischemic stroke.

A therapeutically effective amount of the combination can provide prevention, amelioration or reduction of a symptom related to cerebrovascular injury.

A cerebrovascular injury can be one or more of: neuronal damage, residual clot persistence, microvascular hypoperfusion, blood-brain-barrier leakage, and ischemic lesion expansion.

A therapeutically effective amount of the mammalian exosomes can range from 0.0001 μg/kg to 1.0 mg/kg of a subject's body weight, or from 0.0007 μg/kg to 7.0 mg/kg of a subject's body weight.

A therapeutically effective amount of tPA can ranges from 0.6 mg/kg to 7.0 mg/kg of a subject's body weight, or from 0.6 mg/kg to 1.0 mg/kg of a subject's body weight.

A mammalian exosome can be an exosome containing at least one of the miRNAs miRNA-19a, miRNA-21, or miRNA-146a. miRNA-146a can be selectively overexpressed in a mammalian exosome over a level of miRNA-146a expression in naïve or control exosomes. Mammalian exosomes can be enriched with miR-146a. The concentration of miR-146a in the mammalian exosomes can be at least about twice, or about three times, or about four times, or about five times, or about six times, or about seven times, or about eight times, or about nine times, or about 10 times, or about 100 times, or about 1000 times the concentration of miR-146a in naïve or control exosomes.

Mammalian exosomes can be derived or isolated from stem cells, mesenchymal stromal cells, umbilical cord cells, endothelial cells, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, human embryonic kidney (HEK) cells or mastocytes.

A therapeutically effective amount of the mammalian exosomes comprises from about 1×10⁷ to about 1×10¹⁷ exosomes, or from about 1×10¹² to about 1×10¹⁵ exosomes.

Mammalian exosomes can be administered by intravenous injection, intra-arterial injection, subcutaneous injection, intramuscular injection, intraperitoneally, stereotactically, intranasally, mucosally, intravitreally, intrastriatally, or intrathecally. Mammalian exosomes can be administered by intravenous injection.

A therapeutically effective amount of a combination of mammalian exosomes and tPA can be administered after the onset of stroke symptoms. A therapeutically effective amount of a combination of mammalian exosomes and at least one thrombolytic agent can be administered after the onset of stroke symptoms. Mammalian exosomes can be administered after the onset of stroke symptoms. Mammalian exosomes can be administered 1 minute to 9 hours after the onset of stroke symptoms. Mammalian exosomes can be administered about 10 minutes to about 6 hours after the occurrence of stroke.

Mammalian exosomes can be administered about 10 minutes to about 12 hours after the occurrence of stroke, or about 10 minutes to about 24 hours after the occurrence of stroke, or about 10 minutes to about 48 hours after the occurrence of stroke, or about 10 minutes to about 36 hours after the occurrence of stroke, or about 10 minutes to about 72 hours after the occurrence of stroke, or about 10 minutes to about 4 days after the occurrence of stroke, or about 10 minutes to about 5 days after the occurrence of stroke, or about 10 minutes to about 6 days after the occurrence of stroke, or about 10 minutes to about 7 days after the occurrence of stroke, or about 10 minutes to about 8 days after the occurrence of stroke, or about 10 minutes to about 9 days after the occurrence of stroke, or about 10 minutes to about 10 days after the occurrence of stroke.

tPA can be administered after the onset of stroke symptoms. tPA can be administered about 1 minute to about 9 hours after the onset of stroke symptoms.

Mammalian exosomes and tPA can be administered concomitantly or sequentially. Administration of mammalian exosomes can increase the therapeutic window in which tPA may be administered. An increase of the therapeutic window in which tPA may be administered after the onset of stroke symptoms can be 6 hours to 12 hours.

An administration of a therapeutically effective combination can provide one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) diminishes leakage of the blood-brain-barrier, and (f) attenuates infarct expansion. An administration of a therapeutically effective combination can provide an extension of the therapeutic window for administering tPA to cause a measurable thrombolytic effect in the subject having the stroke.

An at least one thrombolytic agent can be administered after the onset of stroke symptoms. An at least one thrombolytic agent can be administered about 1 minute to about 9 hours after the onset of stroke symptoms.

Mammalian exosomes and An at least one thrombolytic agent can be administered concomitantly or sequentially. Administration of mammalian exosomes can increase the therapeutic window in which an at least one thrombolytic agent may be administered. An increase of the therapeutic window in which an at least one thrombolytic agent may be administered after the onset of stroke symptoms can be 6 hours to 12 hours.

An administration of a therapeutically effective combination can provide one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) diminishes leakage of the blood-brain-barrier, and (f) attenuates infarct expansion. An administration of a therapeutically effective combination can provide an extension of the therapeutic window for administering tPA to cause a measurable thrombolytic effect in the subject having the stroke.

A thrombectomy can be performed with a stent retriever, coil retriever, aspiration device, balloon maceration device, hydrodynamic device, acoustic energy device, spinning brush, or spinning wire device.

A therapeutically effective amount of mammalian exosomes can be administered, and a thrombectomy can be performed, after the onset of stroke symptoms. A thrombectomy can be performed after the onset of stroke symptoms. A thrombectomy can be performed 1 minute to 24 hours after the onset of stroke symptoms. Mammalian exosomes can be administered, and a thrombectomy can be performed, concomitantly or sequentially.

An administration of a therapeutically effective amount of mammalian exosomes and the performance of a thrombectomy in combination provides one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) diminishes leakage of the blood-brain-barrier, (f) attenuates infarct expansion, (g) reduces prothrombotic procoagulant vascular conditions, (h) reduces vascular and/or cerebral brain cell inflammation, and (i) reduces prothrombotic procoagulant vascular conditions and vascular and subsequent cerebral brain cell inflammation.

An administration of a therapeutically effective amount of mammalian exosomes in combination with tPA, and the performance of a thrombectomy in combination provides one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) diminishes leakage of the blood-brain-barrier, (f) attenuates infarct expansion, (g) reduces prothrombotic procoagulant vascular conditions, (h) reduces vascular and/or cerebral brain cell inflammation, and (i) reduces prothrombotic procoagulant vascular conditions and vascular and subsequent cerebral brain cell inflammation.

An administration of a therapeutically effective amount of mammalian exosomes in combination with tPA provides one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) diminishes leakage of the blood-brain-barrier, (f) attenuates infarct expansion, (g) reduces prothrombotic procoagulant vascular conditions, (h) reduces vascular and/or cerebral brain cell inflammation, and (i) reduces prothrombotic procoagulant vascular conditions and vascular and subsequent cerebral brain cell inflammation

An administration of a therapeutically effective combination can provide an extension of the therapeutic window for administering tPA to cause a measurable thrombolytic effect in the subject having the stroke.

An administration of a therapeutically effective combination can provide an extension of the therapeutic window for administering an at least one thrombolytic agent to cause a measurable thrombolytic effect in the subject having the stroke.

Mammalian exosomes containing or enriched with miRNAs miRNA-19a, miRNA-21, or miRNA-146a can comprise human endothelial cells, or endothelial cell progenitor cells. Human endothelial cells can comprise primary or tissue cultured cerebral endothelial cells (CEC).

The methods of the present disclosure can further comprises: (a) administration of a therapeutically effective dose of tPA prior to, or subsequent to the administration of the mammalian exosomes, or (b) a thrombectomy procedure performed prior to, or subsequent to the administration of the mammalian exosomes.

The present disclosure provides a composition comprising mammalian exosomes enriched with at least one miRNAs selected from the group consisting of: miRNA-19a, miRNA-21, and miRNA-146a.

miRNA-146a can be selectively overexpressed in the mammalian exosomes over a level of miRNA-146a expression in naïve or control exosomes. Mammalian exosomes can be human exosomes derived from a human cell culture. Human exosomes can be derived from human endothelial cells, or human endothelial cell progenitor cells.

The present disclosure provides a composition comprising a modified population of cells, wherein the cells overexpress miR-146a over the level of expression of said miRNA-146a in naïve or control cells. Cells can be modified through transient transfection with a miRNA-146a mimic. Control cells can be cells that have been transfected with a mimic control that does not express miRNA-146a. Cells can be human endothelial cells, or human endothelial cell progenitor cells.

Cells can overexpress miR-146a by least 2 fold, or by at least 3 fold, or by at least 5 fold, or by at least 10 fold as compared to the level of expression of said miRNA-146a in naïve or control cells. Cells can overexpress miR-146a by at least 5%, or by at least 10%, or by at least 25%, or by at least 50% when compared to the level of expression of said miRNA-146a in naïve or control cells.

The present disclosure provides a composition comprising a plurality of mammalian exosomes, wherein the mammalian exosomes comprise miR-146a. Mammalian exosomes can be enriched with miR-146a. The concentration of miR-146a in the mammalian exosomes can be at least about twice, or at least about three times, or at least about four times, or at least about five times, or at least about six times, or at least about seven time, or at least about eight times, or at least about nine times, or at least about 10 times, or at least about 100 times the concentration of miR-146a in naïve or control exosomes.

Mammalian exosomes can be derived from a mammalian cell. Mammalian exosomes can be derived or isolated from stem cells, mesenchymal stromal cells, umbilical cord cells, endothelial cells, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, human embryonic kidney (HEK) cells, spindle neurons, microglia, or mastocytes. Mammalian exosomes can be derived from human endothelial cells or human endothelial cell progenitor cells that have been transfected with a miRNA-146a mimic. Mammalian exosomes can be derived from human embryonic kidney (HEK) cells that have been transfected with a miRNA-146a mimic.

The present disclosure provides a composition comprising mammalian exosomes enriched with at least one miRNAs selected from the group consisting of: miRNA-19a, miRNA-21, and miRNA-146a. Mammalian exosomes can overexpress miR-146a by at least 2 fold, or by at least 3 fold, or by at least 5 fold, or by at least 10 fold as compared to a level of expression of said miRNA-146a in naïve or control cells. Mammalian exosomes can overexpress miR-146a by at least 5%, or by at least 10%, or by at least 25%, or by at least 50%, or by at least when compared to the level of expression of said miRNA-146a in naïve or control cells. Mammalian exosomes can overexpress miR-146a by at least 10%, or by at least 25%, or by at least 50% when compared to the level of expression of said miRNA-146a in naïve or control cells.

The present disclosure provides a kit comprising at least one therapeutically effective dose of mammalian exosomes of the present disclosure, at least one therapeutically effective dose of tPA, and a package insert comprising instructions for using the mammalian exosomes and tPA in combination to treat stroke in a subject in need thereof.

The present disclosure provides a kit comprising at least one therapeutically effective dose of mammalian exosomes of the present disclosure, at least one therapeutically effective dose of tPA, and a package insert comprising instructions for using the mammalian exosomes and tPA in combination to treat or prevent a cerebrovascular injury in a subject in need thereof.

The present disclosure provides a kit comprising at least one therapeutically effective dose of mammalian exosomes of the present disclosure, at least on therapeutically effective dose of tPA, and a package insert comprising instructions for using the mammalian exosomes and tPA in combination to treat or prevent secondary thrombosis in downstream brain microvessels in a subject.

The present disclosure provides a kit comprising at least one therapeutically effective dose of mammalian exosomes of the present disclosure, at least one therapeutically effective dose of tPA, and a package insert comprising instructions for using the mammalian exosomes and tPA in combination to treat or prevent a blood brain barrier impairment in a subject.

The present disclosure provides a kit comprising at least one therapeutically effective dose of mammalian exosomes of the present disclosure, at least one therapeutically effective dose of tPA, and a package insert comprising instructions for using the mammalian exosomes and tPA in combination to treat or prevent a cerebrovascular injury.

Cerebrovascular injury can be neuronal damage, residual clot persistence, microvascular hypoperfusion, blood-brain-barrier leakage, or ischemic lesion expansion. Cerebrovascular injury can be the presentation of symptoms consistent with is neuronal damage, residual clot persistence, microvascular hypoperfusion, blood-brain-barrier leakage, or ischemic lesion expansion.

The present disclosure provides a kit comprising at least one therapeutically effective dose of mammalian exosomes of the present disclosure, at least one therapeutically effective dose of tPA, at least one thrombectomy device, and a package insert comprising instructions for using the mammalian exosomes, tPA and the thrombectomy device in combination to treat or prevent a cerebrovascular injury.

The methods of the present disclosure can comprise any of the compositions or kits of the present disclosure.

In one aspect, the invention relates to a composition comprising a modified population of cells, wherein the cells overexpress miR-146a over the level of expression of said miRNA-146a in naïve or control cells. In one embodiment, the cells have been modified through transfection with a miRNA-146a mimic. In another embodiment, the control cells do not express miRNA-146a. In some embodiments, the cells are human endothelial cells, or human endothelial cell progenitor cells. In other embodiments, the cells are cerebral endothelial cells or mesenchymal stromal cells.

In another aspect, the invention relates to a composition comprising a population of mammalian exosomes enriched with miR-146a over the level of said miRNA-146a expression in naïve or control exosomes. In one embodiment, the exosomes are derived from human endothelial cells, or human endothelial cell progenitor cells that have been transfected with an miRNA-146a mimic.

In one embodiment, the cells provided herein overexpress miR-146a by at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 14 fold, at least 15 fold when compared to the level of expression of said miRNA-146a in naïve or control cells. In another embodiment, the cells provided herein overexpress miR-146a by at least 1%, at least 2%, at least 3%, at least 4%, at least 5%, at least 6%, at least 7%, at least 8%, at least 9%, at least 10%, at least 15%, at least 20%, at least 25%, at least 30%, at least 40%, at least 50%, at least 60%, at least 70%, at least 80%, at least 90%, at least 100%, at least 200%, at least 300% when compared to the level of expression of said miRNA-146a in naïve or control cells.

In one aspect, the invention relates to a composition comprising mammalian exosomes (also referred to herein as “exosomes”) enriched with at least one miRNAs selected from the group consisting of: miRNA-19a, miRNA-21, and miRNA-146a. In one embodiment, the miRNA-146a is selectively overexpressed in the mammalian exosomes over the level of miRNA-146a expression in naïve or control exosomes.

In one embodiment, the mammalian exosomes provided herein overexpress miR-146a by at least 2 to 10 fold over the level of said miRNA-146a expression in naïve or control exosomes. In another embodiment, the mammalian exosomes provided herein overexpress miR-146a by at least 10 to 50% over the level of the miRNA-146a expression in naïve or control exosomes.

In another aspect, the invention relates to a method for the treatment of stroke, the method comprising administering a therapeutically effective amount of a combination comprising mammalian exosomes and Tissue Plasminogen Activator (tPA) to a subject in need thereof. In one embodiment, the therapeutically effective amount of the combination provides prevention, amelioration or reduction of a symptom related to cerebrovascular injury.

In one aspect, the invention relates to a method for the treatment and prevention of cerebrovascular injury in a subject who has suffered a stroke, the method comprising administering a therapeutically effective amount of a combination of mammalian exosomes and Tissue Plasminogen Activator (tPA) to a subject in need thereof.

In another aspect, the invention relates to a method for the treatment and prevention of cerebrovascular injury in a subject who has suffered a stroke, the method comprising administering a therapeutically effective combination of mammalian exosomes along with performing a thrombectomy to a subject in need thereof.

In one embodiment, the cerebrovascular injury is neuronal damage, residual clot persistence, microvascular hypoperfusion, blood-brain-barrier leakage, or ischemic lesion expansion. In another embodiment, the stroke is an ischemic stroke. In yet another embodiment, the subject is a human.

In one aspect, the invention relates to a method for treating or preventing secondary thrombosis in downstream brain microvessels, and blood brain barrier impairment in a subject having suffered a stroke, the method comprising administering to the subject in need thereof, a therapeutically effective amount of mammalian exosomes containing or enriched with miRNAs miRNA-19a, miRNA-21, and miRNA-146a.

In one embodiment, the mammalian exosomes containing or enriched with miRNAs miRNA-19a, miRNA-21, or miRNA-146a comprise human endothelial cells, or endothelial cell progenitor cells. In another embodiment, the miRNA-146a is selectively overexpressed in the mammalian exosomes over the level of miRNA-146a expression in naïve or control exosomes.

Other features and advantages of the present invention will become apparent from the following detailed description examples and figures. It should be understood, however, that the detailed description and the specific examples while indicating embodiments of the invention are given by way of illustration only, since various changes and modifications within the spirit and scope of the invention will become apparent to those skilled in the art from this detailed description. Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF DRAWINGS

FIG. 1A shows representative images of CD31⁺ cultured primary cerebral endothelial cells. FIG. 2B shows representative images of ZO1⁺ cultured primary cerebral endothelial cells. FIG. 1C shows a TEM image of endothelial exosomes, scale bar=100 nm. FIG. 1D shows the results of nanopore-based measurement with qNano to measure the distribution of exosomal particles. FIG. 1E shows western blot analysis of the presence of endothelial protein CD31 and exosomal proteins Alix and CD63 in the CEC-exo, but not in supernatant.

FIG. 2A shows a series of graphs showing neurological function measured by modified neurological severity score (mNSS), adhesive removal and foot-fault tests in rats treated with saline, tPA, and the combination of CEC-exos and tPA after MCAO. FIG. 2B is a graph showing the incidence of gross hemorrhage. FIG. 2C is a series of images and graphs showing representative infarction on H&E-stained coronal sections and quantitative data of infarct volume of these rats 7 days after MCAO. All data presented as Mean±SE.

FIG. 3 is a graph showing the effect of CEC-exosomes on expansion of ischemic lesion volume as measured by ADC and T2.

FIG. 4A is a series of images showing brain sections (H&E) of infarction in young adult male and female rats after MCAO. FIG. 2B is a graph showing the quantitative data of infarction in young adult male and female rats after MCAO.

FIG. 5A shows a schematic representation of the model of embolic MCAO. FIG. 5B shows representative images of the residue embolus within the MCA and the intracranial segment of internal carotid artery ICA (the boxed area in FIG. 5A) in rats treated with saline, tPA, and the combination of CEC-exosomes and tPA at 24 h after MCAO. FIG. 5C shows coronal section of microvessels perfused with FITC-dextran in rats treated with saline, tPA, and the combination of CEC-exosomes and tPA at 24 h after MCAO. FIG. 5D is a graph showing residue clot size in rats treated with saline, tPA, and the combination of CEC-exosomes and tPA at 24 h after MCAO. FIG. 5E is a graph showing percentage of vascular areas perfused with FITC-dextran in rats treated with saline, tPA, and the combination of CEC-exosomes and tPA at 24 h after MCAO.

FIG. 6A shows representative coronal sections of Evans blue within brain and a graph quantifying the Evans blue staining. FIG. 6B shows representative confocal microscopic images of fibrin deposition (green) localized to outside of blood vessels (red) and a graph showing the quantitative data of parenchymal fibrin deposition in ischemic rats treated with saline, tPA alone, and CEC-exos in combination with tPA.

FIG. 7A shows representative images of MRA, CBF, ADC, and T2 from rats treated with tPA alone or tPA in combination with CEC-exosomes initiated 4 h after eMCAO. Before the treatments, the right MCA was occluded (arrows, before), CBF was reduced and ischemic lesion was evident in the territory supplied by occluded MCA. However, recanalization of the occluded MCA was detected at 2 h and 24 h after the combination treatment, which was associated with increased downstream CBF and reduced infarction (ADC, T2), whereas these changes were not detected in the rat treated with tPA alone. FIG. 7B is a graph showing infarct volume and FIG. 7C is a graph showing low CBF volume in rats treated with tPA alone or tPA in combination with CEC-exosomes initiated 4 h after eMCAO

FIG. 8A is a TEM images of emboli in cortical ischemic lesions of rates treated with tPA 24 h after MCAO. FIG. 8B is a TEM image of emboli in cortical ischemic lesions of rates treated with tPA 24 h after MCAO. FIG. 8C is a TEM image of downstream microvessels in cortical ischemic lesions of rates treated with tPA 24 h after MCAO. FIG. 8D is a TEM image of neurons in cortical ischemic lesions of rates treated with tPA 24 h after MCAO. FIG. 8E is a TEM image of emboli in cortical ischemic lesions of rates treated with tPA in combination with CEC-exosomes 24 h after MCAO. FIG. 8F is a TEM image of downstream microvessels in cortical ischemic lesions of rates treated with tPA in combination with CEC-exosomes 24 h after MCAO. FIG. 8G is a TEM image of neurons in cortical ischemic lesions of rates treated with tPA in combination with CEC-exosomes 24 h after MCAO. FIG. 8H is a TEM image of neurons in cortical ischemic lesions of rates treated with tPA in combination with CEC-exosomes 24 h after MCAO. Dense fibrin bundles (FIG. 8A) adhered with active granular platelets (FIG. 8B, arrowheads), red blood cell trapped capillary (FIG. 8C) and dead neuron (FIG. 8D) were present in rats treated with tPA, whereas few fibrin bundles (FIG. 8E), open lumen of downstream capillary covered by a pericyte (FIG. 8F) with intact tight junction (FIG. 8F, arrow), intact neurons (FIG. 8G), and synaptic complex at axon terminal (FIG. 8H arrow) were detected in rats treated with tPA and CEC-exosomes.

FIG. 9A is a series of graphs showing levels of miRNAs in cerebral endothelial cells harvested from microvessels of non-ischemic rats or ischemic rats treated with saline, tPA or tPA+CEC-exosomes 24 h after MCAO. FIG. 9B is a series western blot images showing levels of proteins in cerebral endothelial cells harvested from microvessels of non-ischemic rats or ischemic rats treated with saline, tPA or tPA+CEC-exosomes 24 h after MCAO. FIG. 9B is a series of graphs showing levels of proteins in cerebral endothelial cells harvested from microvessels of non-ischemic rats or ischemic rats treated with saline, tPA or tPA+CEC-exosomes 24 h after MCAO. RT-PCR data (FIG. 9A) show levels of miR-21 and -146a. FIG. 9B and FIG. 9C show representative Western blot and quantitative data of ICAM1, PAI1, TF, TLR4, NF-κB, and ZO1. vs non-stroke and saline, respectively. n=3 rats/group. *p<0.05 and #p<0.05

FIG. 10 shows a series of western blot images (left) and quantitation of the western blot images (right) of ICAM1, PAI1, and TF levels in plasma of non-ischemic rats or ischemic rats treated with saline, tPA or tPA+CEC-exos 24 h after MCAO. n=3 rats/group. *p<0.05 vs non-stroke.

FIG. 11A is an image of CECs transfected with CD63-GFP. FIG. 11B is a Western blot image of exosomes without GFP (Exo) and with CD63-GFP (Exo-GFP). FIG. 11C shows orthogonal confocal images showing GFP signals in cerebral endothelial cells 4 h after administration (IV) of CEC-exosomes/CD63-GFP. FIG. 11D shows orthogonal confocal images showing GFP signals in neurons 4 h after administration (IV) of CEC-exosomes/CD63-GFP.

FIG. 12A shows a TEM image and western blot analysis of clot-exosomes and assayed for the presence of exosomal proteins Alix and CD63. FIG. 12B shows an in vitro BBB assay and quantitative data of BBB permeability in endothelial cells treated with clot-exosomes alone or in the presence of CEC-exosomes (n=5/group). FIG. 12C shows a signaling network of miR-19a, -21 and -146a and their direct and indirect target genes in regulation of endothelial cell function (including promoting vascular injury and, thrombogenicity), based on Ingenuity Pathway Analysis (IPA).

FIG. 13 is a series of representative western blots images (left) and quantitation of the western blots of various individual proteins in endothelial cells treated with patient-derived exosomes (clot-exos) or with patient-derived exosomes in combination with CEC-exos (clot-exos/CEC-exos). n=3/group.

FIG. 14A-D show TEM images showing how an embolic MCAO model is performed placing a clot to the origin of the MCA that the placement of the clot induces platelet aggregation and PAI-1 upregulation within the clot and between border of the clot and the blood luminal surface. Scale Bar=40 μm for FIG. 14A and FIG. 14C, 10 μm for FIG. 14B and FIG. 14D.

FIG. 15A shows representative images of brain infarction 7 days after transient MCAO. FIG. 15B shows quantitative infarct volumes 7 days after transient MCAO. FIG. 15C shows neurological outcomes measured by mNSS, Adhesive remove test, and foot-fault test.

FIG. 16A is a graph showing infarct volume at baseline and 7 days after 1 hour transient MCAO. FIG. 16B is a series of graphs showing neurological outcomes at baseline and 7 days after 1 hour transient MCAO.

FIG. 17A shows western blot data of exosomal protein levels from individual subjects. N is a healthy subject and the individual stroke subjects are numbered from 1 to 9. FIG. 17B is a series of data plots of results of each protein and individual NIH stroke scores. Scores at discharge were subtracted from scores obtained prior to the thrombectomy. FIG. 17C shows correlation results of each protein and individual NIH stroke scores.

FIG. 18A shows a TEM image and western blot analysis of clot-exos and exosomal proteins Alix and CD63. FIG. 18B is a schematic showing an in vitro BBB permeability assay (B). FIG. 18C is a graph showing the quantitative data of BBB permeability in endothelial cells treated with CEC-exosomes alone (Endo exos), stroke patient derived exosomes alone (Clot exos), or stroke patient derived exosomes along with CEC-exos (Endo exos with clot exos).

FIG. 19A is a graph showing quantitative RT-PCR analysis of levels of miR-146a, -125b, and 18a in human primary cerebral endothelial cells (hBMVs) after transfection with miR-146a mimics and their negative control. FIG. 19B is a graph showing quantitative RT-PCR analysis of levels of miR-146a, -125b, and 18a in CEC-exosomes after transfection with miR-146a mimics and their negative control. FIG. 19C is a graph showing quantitative data of an in vitro BBB permeability assay demonstrating the effect tailored CEC-exos miR-146a have on BBB leakage. FIG. 19C is a graph showing quantitative data of an in vitro BBB permeability assay demonstrating the effect tailored MSC-exos-miR-146a have on BBB leakage.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Each example and embodiment of the disclosure is to be applied mutatis mutandis to each and every other example or embodiment unless specifically stated otherwise.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any of said steps or features.

The present disclosure is not to be limited in scope by the specific examples described herein, which are intended for the purpose of exemplification only. Functionally-equivalent compositions and methods are clearly within the scope of the disclosure.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, formulation and medical treatments in cardiology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); Textbook of Interventional Cardiology, 7th Edition, Authors: Eric J. Topol & Paul S. Teirstein; and Animal Cell Culture: Practical Approach, Third Edition (John R. W. Masters, ed., 2000), ISBN 0199637970, whole of text; each of these references are incorporated herein by reference in their entireties.

The present disclosure provides methods for the treatment of stroke that involves administering a therapeutically effective amount of a combination comprising mammalian extracellular vesicles (which include exosomes and/or microvesicles) and Tissue Plasminogen Activator (tPA) to a subject in need thereof.

As used herein, the term “treat” or “treating” or “treatment” refers to clinical intervention designed to alter the natural course or outcome of a pathological condition affecting an individual undergoing said treatment. Desirable effects of treatment include decreasing the rate of progression, ameliorating or palliating the pathological state, and remission or improved prognosis of a particular disease, disorder, or condition. For example, an individual is successfully “treated”, if one or more symptoms associated with a particular disease, disorder, or condition are diminished, mitigated or eliminated. Furthermore, the terms “to treat” or “treatment” according to this disclosure include the treatment of symptoms of cerebrovascular injury, disorder or disease, the prevention or the prophylaxis of the symptoms of cerebrovascular injury resulting from ischemic stroke, the prevention or prophylaxis causing the symptoms of cerebrovascular injury, disorder or disease, as well as the prevention or the prophylaxis of the consequences causing the symptoms.

“Prevent” refers to delaying or forestalling the onset or development of a disease, development of one or more symptoms associated with such disease, disorder, or condition for a period of time from minutes to indefinitely. Prevent also means reducing risk of developing a disease, disorder, or condition. “Prevent” or “preventing” or “prevention” shall be taken to mean administering an amount of mammalian exosomes and/or microvesicles, or cargo constituents from exosomes and/or microvesicles, or soluble factors derived therefrom, along with tPA and/or performing a thrombectomy procedure, to effectuate the stopping or hindering or delaying of the development or progression of a disease, disorder or condition, and/or the corresponding symptoms e.g. cerebrovascular injury following a stroke. “Prevent” or “preventing” or “prevention” refers to prevention or delay of the onset of the disease, disorder or condition, and/or a decrease in the level of discomfort, general malaise, or persistence of the symptoms of a given disease, disorder, or condition, in a subject relative to the symptoms that would develop and/or persist in the absence of the methods of the invention. The prevention can be complete, e.g., the total absence of disease, disorder, or conditions, and/or its corresponding symptoms. The prevention can also be partial, such that the occurrence of the disorder or disease symptoms in a subject is less than that which would have occurred without the present method.

As used herein, the term “effective amount” or “therapeutically effective amount” means the amount of mammalian exosomes and/or microvesicles, tPA, and/or a combination thereof, sufficient to effectuate a desired physiological outcome in an individual in need of the foregoing items. The effective amount can vary among individuals depending on the health and physical condition of the individual to be treated, the taxonomic group of the individuals to be treated, the formulation of the composition, assessment of the individual's medical condition, and other relevant factors. Moreover, as used herein, the term “therapeutically effective amount” refers to the minimum concentration required to effect a measurable improvement of a particular disease, disorder, or condition, for example, symptoms, and comorbidity associated with stroke. Accordingly, the therapeutically effective amount may vary based on factors such as the disease state (e.g., size, composition, and age of the thrombus; specific arteries involved), age, sex, and/or weight of the patient, along with the ability of the mammalian exosomes and/or microvesicles to act in concert with tPA and/or thrombectomy to elicit a desired response in the individual. A therapeutically effective amount is also one in which any toxic or detrimental effects of tPA administration or thrombectomy procedure are outweighed by the therapeutically beneficial effects.

A “suboptimal amount” is an amount that is below the optimal or standard minimum concentration required to effect a measurable improvement of a particular disease, disorder, or condition. In some embodiments, tPA by itself (i.e., when not used in combination with mammalian exosomes) may be a suboptimal amount; however, in some embodiments, when a suboptimal amount of tPA is used with mammalian exosomes, the suboptimal amount of tPA may be a therapeutically effective amount.

As used herein, the term “therapeutically effective combination” or “therapeutically effective amount of a combination” (used synonymously) refers to the result or product of combining two or more agents, elements, drugs, and/or treatments (e.g., mammalian exosomes and/or microvesicles and tPA, or mammalian exosomes and/or microvesicles and thrombectomy), the combination of which results in at least the minimum combined concentration required to effect a measurable improvement of a particular disease, disorder, or condition, e.g. cerebrovascular injury as a result of stroke. The therapeutically effective combination may vary based on factors such as the disease state (e.g., size, composition, and age of the thrombus; specific arteries involved), age, sex, and/or weight of the patient, along ability of the mammalian exosomes in concert with tPA and/or thrombectomy to elicit a desired response in the individual. A therapeutically effective combination is also one in which any toxic or detrimental effects of the mammalian exosomes are outweighed by the therapeutically beneficial effects.

“About” means within plus or minus (±) 10% of a value. For example, if it is stated, “a marker may be increased by about 50%”, it is implied that the marker may be increased between 45%-55%, inclusive of the endpoints and all integers or fractions thereof between the stated ranges.

“Amelioration” refers to a lessening of at least one indicator, sign, or symptom of an associated disease, disorder, or condition. The severity of indicators can be determined by subjective or objective measures, which are known to those skilled in the art.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Parenteral administration” means administration by a manner other than through the digestive tract. Parenteral administration includes topical administration, subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Patient” or “Subject” are used interchangeably and for the purposes of the present disclosure includes humans and other animals, particularly mammals, and other organisms. Thus the methods are applicable to both human therapy and veterinary applications. More specifically, the patient is a mammal, and in some embodiments, the patient or subject is human.

“Pharmaceutical composition” means a mixture of substances suitable for administering to an individual. For example, a pharmaceutical composition can comprise one or more active agents and a sterile aqueous solution.

“Pharmaceutically acceptable carrier” means a medium or diluent that does not interfere with the structure or function of the oligonucleotide. Certain, of such carries enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection or infusion. For example, a pharmaceutically acceptable carrier can be a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Pharmaceutically effective amount” for purposes herein is thus determined by such considerations as are known in the art, and may also include “therapeutically effective amounts” (also used synonymously) which is broadly used herein to mean an amount of mammalian exosomes, tPA, and/or the performance of a thrombectomy procedure, that when administered to a patient, ameliorates, diminishes, improves or prevents a symptom of cardiovascular disorder or disease in a patient who has suffered a stroke, and who may or may not have a glucose metabolism disorder. The amount of mammalian exosomes, tPA, and/or the performance of a thrombectomy procedure described herein, or their internal components which constitutes a “therapeutically effective amount” where applicable, will vary depending on the agent density, the disease state and its severity, the age of the patient to be treated, and the like.

“Prophylactically effective amount” or “prophylactic amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the term “suffer” as in “suffered a stroke” or “suffer a stroke” means a subject or patient who has deprived blood supply to the brain, or has compressed brain tissue, owing to an obstruction of blood vessels, arterial stenosis, or ruptured blood vessels, and consequently or coincidentally has a one or more of the cerebrovascular injuries and/or corresponding symptoms enumerated below, or is likely to develop one or more of the cerebrovascular injuries and/or corresponding symptoms enumerated below.

As used herein, the term “stroke” shall be taken to mean loss of brain function(s), usually rapidly developing, that is due to a disturbance in blood flow to the brain or brainstem. The term stroke shall be taken to mean a condition where the brain is deprived of an adequate supply of blood, and/or the amount of oxygen and/or nutrients. The term stroke includes ischemic stroke, acute ischemic stroke, or thrombotic stroke; however, the term stroke, as used herein, does not refer to hemorrhagic stroke. Ischemic stroke can occur due to ischemia (i.e., lack of blood), as a result of thrombosis or embolism. In one example, the loss of brain function is accompanied by neuronal cell death. In one example, the stroke is caused by a disturbance or loss of blood from to the cerebrum or a region thereof. In one example, a stroke is a neurological deficit of cerebrovascular cause that persists beyond 24 hours or is interrupted by death within 24 hours (as defined by the World Health Organization). Persistence of symptoms beyond 24 hours separates stroke from Transient Ischemic Attack (TIA), in which symptoms persist for less than 24 hours. Symptoms of stroke include hemiplegia (paralysis of one side of the body); hemiparesis (weakness on one side of the body); muscle weakness of the face; numbness; reduction in sensation; altered sense of smell, sense of taste, hearing, or vision; loss of smell, taste, hearing, or vision; drooping of an eyelid (ptosis); detectable weakness of an ocular muscle; decreased gag reflex; decreased ability to swallow; decreased pupil reactivity to light; decreased sensation of the face; decreased balance; nystagmus; altered breathing rate; altered heart rate; weakness in sternocleidomastoid muscle with decreased ability or inability to turn the head to one side; weakness in the tongue; aphasia (inability to speak or understand language); apraxia (altered voluntary movements); a visual field defect; a memory deficit; hemineglect or hemispatial neglect (deficit in attention to the space on the side of the visual field opposite the lesion); disorganized thinking; confusion; development of hypersexual gestures; anosognosia (persistent denial of the existence of a deficit); difficulty walking; altered movement coordination; vertigo; disequilibrium; loss of consciousness; headache; and/or vomiting.

Typically, there are two categories of stroke: ischemic and hemorrhagic (see F. H. Kobeissy, editor: Brain Neurotrauma: Molecular, Neuropsychological, and Rehabilitation Aspects (2015), Boca Raton, Fla., CRC Press/Taylor & Francis); however, as mentioned above, stroke refers to ischemic stroke and/or acute ischemic stroke. Ischemic stroke occurs when the brain's blood supply is restricted due to obstruction of blood vessels or arterial stenosis. Ischemic stroke results in brain cells being deprived of oxygen and energy. There are two main categories of ischemic stroke: thrombotic and embolic stroke. During a thrombotic stroke, a blood clot forms at the occlusion site; alternatively, in embolic stroke, the clot forms at a distant artery and subsequently travels to the occlusion site.

As used herein, the term “cerebrovascular injury” shall be taken to mean a condition and/or symptom including, but not limited to, neuronal damage, death, and/or degeneration; residual clot persistence; microvascular hypoperfusion; ischemic lesion expansion; congested and/or engorged grey matter; gliosis; glial scarring; hypertrophy of astrocytes, microglia, and/or oligodendrocytes; disrupted blood-brain-barrier; dysregulation of the blood brain barrier; dysregulation of blood pressure; disruption of cerebral blood flow; reduced cerebral blood perfusion; inhibition of neuronal protein synthesis; disruption of neuronal glucose utilization; tissue acidosis; neuronal electrical failure; brain tissue necrosis; neuronal cell apoptosis; neuronal cell necrosis; neuronal-support cell (e.g., glial cell) apoptosis and/or necrosis; depletion of adenosine triphosphate (ATP) in neurons and/or brain-associated cells; changes in ionic concentrations of sodium, potassium, and calcium in the brain; increased lactate in the brain; brain tissue acidosis; accumulation of oxygen free radicals in the brain; intracellular accumulation of water in the brain; activation of proteolytic processes in neuronal and neuronal-support cells; increase release of glutamate at neuronal synapses, and the downstream activation of glutamate receptors; ionic disruption; increased production of reactive oxygen species; inflammation; loss of structural integrity in the brain; and/or cerebral edema (cytotoxic or vasogenic).

“Neuronal damage” or “neuron damage” or “neuron injury” (used synonymously) refers to damage and/or death to nerve or neuronal cells (e.g., autonomic nerves, sensory nerves, and/or motor nerves) and/or their support cells (e.g., Schwann cells, glia cells, satellite cells, etc.) of the central nervous system (CNS) (e.g., brain or spinal cord) and/or the peripheral nervous system (PNS) (e.g., autonomic, spinal, or cranial neurons). Neuron damage can occur as a result of stroke, or any condition where the neurons are starved of oxygen or nutrients, and can be identified by the reduction of the number of neurons, for example as a result of apoptosis or necrosis; neuron damage can also be identified by a reduction in neuron length (e.g., axons and/or dendrites), or a reduction in expression of neuronal markers such as NSE and KCC2. Neuronal damage is also used herein to describe the effect and/or end result of cerebral infarct (i.e., the death of brain tissue). Biomarkers of neuronal damage include Brain Natriuretic Peptide (BNP); C-reactive Protein (CRP); D-Dimer; elevated fibrinogen levels; Neuron specific enolase (NSE); Copeptin; Glial fibrillary acidic protein (GFAP); Matrix metalloproteinase 9 (MMP9); S100 calcium binding protein B (S100B); and/or the expression of genes involved in oxygen homeostasis, such as HIF-1α which is expressed in response to acute hypoxia, and HIF-2α which is involved in neuronal adaptation to chronic hypoxic stress (see Miguel et al. Preferential activation of HIF-2α adaptive signaling in neuronal-like cells in response to acute hypoxia. PLoS One. 2017; 12(10): e0185664). Symptoms of autonomic nerve damage include an inability to sense pain; hyperhidrosis; anhidrosis; fatigue; faintness; dehydration, including dry eyes and mouth; constipation; incontinence; and/or sexual dysfunction. Motor neuron damage may produce symptoms including weakness; muscle atrophy; fasciculation; and/or paralysis. Sensory nerve damage may produce symptoms such as pain; numbness; hyper or hyposensitivity; paresthesia; and/or ataxia.

“Residual clot persistence” refers to the continued presence of a clot or thrombus that does not dissipate, dissolve, or dismantle, and/or decrease in size after time.

“Microvascular hypoperfusion” refers to the failure of adequate circulation to the vasculature in the brain. Symptoms of microvascular hypoperfusion include hypotension and/or coldness of the skin. The term “microvascular” or “microvasculature” refers to small blood vessels, including arterioles; capillaries; metarterioles; and/or venules.

“Blood-brain-barrier (BBB) leakage” refers to a disruption of the highly regulated vasculature that separates the brain and cerebrospinal fluid (CSF) from the blood, and regulates the simple diffusion of molecules, ions, and cells. The BBB regulates the makeup of brain interstitial fluid via a series of high-resistance, tight junctions between endothelial and astrocytes (see Pardridge et al. Blood-brain barrier: interface between internal medicine and the brain. Ann Intern Med. 1986; 105(1):82).

“Ischemic legion expansion” refers to the growth, development, or expansion of an infarct. The term “infarct” refers to necrotic tissue that has died as a result of inadequate oxygen or nutrient supply, for example, cerebral infarction can occur as a result of an ischemic stroke. During ischemic legion expansion, the size or volume of the infarct will expand or grow, for example, a patient who has suffered a stroke may incur an ischemic legion, manifested as a cerebral infarct, and that ischemic legion may expand if mammalian exosomes, tPA, and/or thrombectomy is not administered.

As used herein, the term “mammalian exosomes” refers to small extracellular vesicles released from cells, which have been shown to carry nucleic acids including microRNAs (Yu et al. Exosomes as miRNA Carriers: Formation-Function-Future, Int J Mol Sci. 2016 December; 17(12): 2028, the disclosure of which is incorporated herein by reference in its entirety). In some embodiments, the extracellular vesicles can be exosomes (size <100 nm), microvesicles (also known as ectosomes, shedding vesicles, microparticles, plasma membrane-derived vesicles, and exovesicles, size <1000 nm), and/or apoptotic bodies (size 1-4 μm) (D. Ha, et al. “Exosomes as therapeutic drug carriers and delivery vehicles across biological membranes: current perspectives and future challenges” (2016) Acta Pharmaceutica Sinica B, Vol 6, Issue 4, p. 287-296, the disclosure of which is incorporated herein by reference in its entirety).

As used herein the term “derived from” shall be taken to indicate that a specified biological product, component or active agent may be obtained from a particular source albeit not necessarily directly from that source. For example, in the context of exosomes and/or microvesicles “derived” from a mammalian cell, this term refers to mammalian exosomes and/or microvesicles that are produced by exosome and/or microvesicle producing mammalian cells, for example, stem cells, mesenchymal stromal cells, umbilical cord cells, endothelial cells, for example, cerebral endothelial cells, Schwann cells, hematopoietic cells, reticulocytes, epithelial cells, monocyte-derived dendritic cells (MDDCs), monocytes, B lymphocytes, antigen-presenting cells, glial cells, astrocytes, neurons, oligodendrocytes, spindle neurons, human embryonic kidney (HEK) cells, microglia, mastocytes, or in vitro cell cultures of any of the foregoing cells. In the foregoing examples, the exemplary mammalian exosomes and/or microvesicles can be isolated from these exemplified cells, or may be cultured from mammalian tissue, for example, mammalian tissue or mammalian cultured cells.

In various embodiments, methods provided herein for the treatment of stroke include administering a therapeutically effective dose comprising a combination of mammalian exosomes and tPA to the subject in need thereof. In some embodiments, the mammalian exosomes can be administered without the addition of any further excipient, carrier or diluent, or in the form of a composition containing the mammalian exosome admixed with one or more excipients, carriers or diluents. In various embodiments, the compositions may include non-pharmaceutical compositions or pharmaceutical compositions approved for administration to a subject, for example a human subject.

In some embodiments, illustrative mammalian exosomes may include exosomes which contain among other proteins (e.g. Alix and/or CD63), growth factors, microRNAs, siRNAs and mRNAs will also contain naked miR-19a microRNA, and/or naked miR-21 microRNA, and/or naked miR-146a microRNA. In some embodiments, the exosomes are non-enriched, in that they are not specifically transformed recombinantly (non-naturally) with an exogenous nucleic acid, for example and nucleic acid, which includes a microRNA, for example, miR-19a microRNA, miR-21 microRNA, and/or miR-146a microRNA. In various embodiments, mammalian exosomes of the present disclosure can include any mammalian exosome that contains or is enriched in miR-19a microRNA, miR-21 microRNA, and miR-146a microRNA. In some embodiments, illustrative mammalian exosomes and/or microvesicles may also include mammalian cell derived exosomes and/or microvesicles that may contain little to no miR-19a microRNA, miR-21 microRNA, or miR-146a microRNA, but which are transformed with miR-19a microRNA, miR-21 microRNA, or miR-146a microRNA coding nucleic acids, for example, plasmids which contain polynucleotides operable to encode miR-19a microRNA, miR-21 microRNA, and/or miR-146a microRNA in the target cell. These exosomes and/or microvesicles are said to be enriched with these microRNAs.

In some embodiments, mammalian cells which are operable to produce exosomes and/or microvesicles of the present invention may include mammalian cells which produce exosomes and microvesicles that contain or are capable of expressing miR-19a, miR-21, or miR-146a microRNA, a vesicle containing miR-19a, miR-21, or miR-146a microRNA, or a particle containing miR-19a, miR-21, or miR-146a microRNA, or agents which induce the expression of miR-19a, miR-21, or miR-146a microRNA in the target cells, or in the target tissue. In various embodiments of the present disclosure, the methods of treatment of stroke may include administering a therapeutically effective dose comprising a combination of mammalian cell exosome cargo and tPA and/or thrombectomy to the subject in need thereof. As used herein “mammalian cell exosome cargo” refers to the internal constituents of the above referenced mammalian cell exosomes, which may include a variety of proteins (e.g. Alix or Tsg101), growth factors, microRNAs, siRNAs and mRNAs, for example, miR-19a, miR-21, or miR-146a microRNAs. In some embodiments, mammalian cell exosome cargo includes internal constituents of exosomes and/or microvesicles that include miR-19a, miR-21, or miR-146a microRNAs among other proteins, and nucleic acids.

In some embodiments, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, human embryonic kidney (HEK) cells, oligodendrocytes, spindle neurons, microglia, or mastocyte cells may be transfected with purified miR-19a, miR-21, or miR-146a. In some embodiments, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, human embryonic kidney (HEK) cells, oligodendrocytes, spindle neurons, microglia, or mastocyte cells may be transfected with an agent that induces miR-19a, miR-21, or miR-146a expression. The nucleotide sequence of the miRNA precursors of miR-19a, miR-21, or miR-146a are shown in SEQ ID NO: 1, 2, and 3, respectively.

In one embodiment, the miRNA precursor nucleotide sequence of miR-19a comprises,

(SEQ ID NO: 1)

GCAGTCCTCT GTTAGTTTTG CATAGTTGCA CTACAAGAAG
AATGTAGTTG TGCAAATCTA TGCAAAACTG ATGGTGGCCT
GC.

In one embodiment, the miRNA precursor nucleotide sequence of miR-21 comprises,

(SEQ ID NO: 2)

TGTCGGGTAG CTTATCAGAC TGATGTTGAC TGTTGAATCT
CATGGCAACA CCAGTCGATG GGCTGTCTGA CA.

In one embodiment, the miRNA precursor nucleotide sequence of miR-146a comprises,

(SEQ ID NO: 3)

CCGATGTGTA TCCTCAGCTT TGAGAACTGA ATTCCATGGG
TTGTGTCAGT GTCAGACCTC TGAAATTCAG TTCTTCAGCT
GGGATATCTC TGTCATCGT.

In one embodiment, the mature miR-19a (hsa-miR-19a) nucleotide sequence comprises, AGUUUUGCAUAGUUGCACUACA (SEQ ID NO: 4).

| (SEQ ID NO: 4) | |
|---|---|
| | AGUUUUGCAUAGUUGCACUACA. |

In one embodiment, the mature miR-21 (hsa-miR-21) nucleotide sequence comprises,

| (SEQ ID NO: 5) | |
|---|---|
| | UAGCUUAUCAGACUGAUGUUGA. |

In one embodiment, the mature miR-146a (hsa-miR-146a) nucleotide sequence comprises,

| (SEQ ID NO: 6) | |
|---|---|
| | UGAGAACUGAAUUCCAUGGGUU. |

In an exemplary method, cells that may or may not naturally produce miR-19a, miR-21, or miR-146a can be transfected or transformed to produce miR-19a, miR-21, or miR-146a, either constitutively or induced by adding an agent to a cell culture to induce production of miR-19a, miR-21, or miR-146a microRNA. For example, microRNA-19a (miR-19a-3p) may be synthesized using the nucleotide sequence 5′-UGUGCAAAUCUAUGCAAAACUGA-3′ (SEQ ID NO: 7). Cerebral Endothelial Cells (CECs) may be transfected and assayed using quantitative real-time polymerase chain reaction (qRT-PCR). CECs may be cultured and transfected with miR-19a-3p according to the manufacturer's instructions using the siPORT NeoFX Transfection Agent (Applied Biosystems Inc.). Briefly, CECs may be grown in DMEM with 10% Fetal Bovine Serum (CellGro) to 80% confluence at 37° C. and 5% CO₂. Adherent cells are washed and trypsinized. Trypsin can be inactivated by re-suspending the cells in DMEM with 10% FBS (Invitrogen). The SiPORT NeoFX transfection agent is diluted in Opti-MEM I medium (Life Technologies) and incubated for 10 minutes at room temperature. miR-19a-3p can be diluted into 50 μL Opti-MEM I medium at a concentration of 30 nM. Diluted microRNA and diluted siPORT NeoFX Transfection agent is mixed and incubated for another 10 minutes at room temperature to allow transfection complexes to form and subsequently dispensed into wells of a clean 6-well culture plate. The CEC suspension is overlaid onto the transfection complexes and gently mixed to equilibrate. Transfected cells are incubated at 37° C. and 5% CO₂ for 24 hours. Cells other than CECs, for example, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, may be used and transfected with one or more polynucleotides for example a vector which is operable to express a microRNA for example, miR-19a, miR-21, and miR-146a that may be packaged into an exosome and/or microvesicle.

In some embodiments, mammalian exosomes can include miR-19a, miR-21, or miR-146a microRNA. In some of these embodiments, methods for isolating miR-19a, miR-21, or miR-146a microRNA are known in the art. In one example, miR-19a, miR-21, or miR-146a microRNA can be produced using general, known molecular biology techniques taking advantage of the nucleotide sequence of miR-19a, miR-21, or miR-146a microRNA as shown in SEQ ID NO: 1, 2, and 3, respectively. For example, a cDNA molecule encoding the complementary sequence of miR-19a, miR-21, or miR-146a microRNA can be cloned into a plasmid and serve as a template for polymerase chain reactions (PCR) for the synthesis of miR-19a, miR-21, or miR-146a which can then be reverse transcribed to RNA. Other methods for isolating miRNA from biological fluids are also known, for example, Lekchnov, E. A., Anal Biochem. (2016), “Protocol for miRNA isolation from biofluids”, 499:78-84. Alternatively, of miR-19a, miR-21, or miR-146a can be synthesized from the nucleotide sequence of miR-19a, miR-21, or miR-146a as provided in SEQ ID NO: 1, 2, and 3, respectively.

In other embodiments, mammalian exosomes also include natural and synthetic nucleic acid vectors (for example, plasmids, cosmids, YACs, and viral vectors) that when expressed in a mammalian cell include a miR-19a, miR-21, or miR-146a nucleic acid sequence (for example, in the case of miR-19a, a polynucleotide containing the nucleotide sequence of SEQ ID NO: 1) and which also contain expression sequences such as promoters, termination signals and other transcription and translation signals operable to express the miR-19a, miR-21, or miR-146a microRNA in its intended cells and tissues to form such exosomes and/or microvesicles.

In some embodiments, mammalian exosomes can contain a combination of miR-19a and miR-21; miR-19a and miR-146a; miR-21 and miR-146a; or miR-19a, miR-21, and miR-146a microRNA. For example, a mammalian cells such as stem cells, mesenchymal stromal cells, umbilical cord cells, endothelial cells, (for example, cerebral endothelial cells), epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, human embryonic kidney (HEK) cells, microglia, or mastocytes which produce exosomes and secrete exosomes and/or microvesicles, may possess exosomes with a mammalian cell exosome cargo that contains at least one of miR-19a, miR-21, or miR-146a microRNA, or all of three of miR-19, miR-21, and miR-146a microRNA, either alone or with other mammalian exosome cargo constituents.

In various embodiments, miR-19a, miR-21, or miR-146a microRNA molecules may be encoded in a target tissue, for example, the vascular endothelium or cells of the heart tissue, e.g. cardiomyocytes by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding a miR-19a, miR-21, or miR-146a microRNA, a vector may encode a targeting molecule. A targeting molecule is one that directs the desired nucleic acid to a particular organ, tissue, cell, or other location in a subject's body.

The term “expression vector” refers to a vector containing a nucleic acid sequence coding for at least part of a gene product capable of being transcribed. Expression vectors can contain a variety of “control sequences,” which refer to nucleic acid sequences necessary for the transcription of an operably linked coding sequence in a particular host organism. In addition to control sequences that govern transcription and translation, vectors and expression vectors may contain nucleic acid sequences that serve other functions as well and are described. There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. They can accommodate up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present disclosure. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Other suitable methods for nucleic acid delivery to effect expression of compositions of the present disclosure are believed to include virtually any method by which a nucleic acid (e.g., RNA, e.g. microRNA, or DNA, including viral and nonviral vectors) can be introduced into an organelle, a cell, a tissue or an organism, as described herein or as would be known to one of ordinary skill in the art. Such methods include, but are not limited to, direct delivery of RNA such as by injection (U.S. Pat. Nos. 5,994,624; 5,981,274; 5,945,100; 5,780,448; 5,736,524; 5,702,932; 5,656,610; 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783; 5,563,055; 5,550,318; 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

In other embodiments, an illustrative mammalian exosome can include exosomes derived from a cell (e.g. a mammalian cell, for example a human cell) that synthesizes and expresses miR-19a, miR-21, and/or miR-146a microRNA, and packages same into an exosome and/or microvesicle. In some embodiments, cells can be administered to treat stroke or the symptoms of stroke by administering a population of mammalian cells that naturally produce and secrete exosomes and/or microvesicles that contain miR-19a, miR-21, and/or miR-146a microRNA, for example, mammalian (for example, human): stem cells, mesenchymal stromal cells, umbilical cord cells, endothelial cells, (for example, cerebral endothelial cells), epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes which produce exosomes and secrete exosomes and/or microvesicles that contain at least one of miR-19a, miR-21, and miR-146a microRNA.

As used herein, the term “Tissue Plasminogen Activator (tPA)” or “tissue-type plasminogen activator” or “PLAT” or “plasminogen activator” or “plasminogen activator, tissue type” (used synonymously herein) refers an endogenous fibrinolytic serine protease enzyme, about 69 kDa large. In some embodiments, an illustrative tPA useful in the methods, compositions, and products described herein has an accession number NM_000930, that mediates fibrinolysis (van Overbeek et al., Plasma tPA-Activity and Progression of Cerebral White Matter Hyperintensities in Lacunar Stroke Patients, PLoS One. 2016; 11(3): e0150740). In some embodiments, an exemplary tPA useful in the methods, compositions and products described herein is 562 amino-acids long, contains three glycosylation sites, and 17 disulfide bridges (Cheviley et al. Impacts of tissue-type plasminogen activator (tPA) on neuronal survival, Front Cell Neurosci. 2015; 9: 415). In some embodiments of the present disclosure, the tPA is a recombinant tPA. tPA can be obtained by inserting a nucleic acid sequence coding for at least part of a tPA gene product capable of being transcribed into a vector (e.g., plasmids, cosmids, and artificial chromosomes such as YACs) and through standard recombinant techniques, express tPA mRNA, translate the tPA mRNA into protein, and purify said protein; the tPA mRNA nucleic acid sequence can be derived from any mammalian species, including but not limited to, Homo sapiens, Bos taurus, Mus musculus, Pan troglodytes, Sus scrofa, Gallus gallus, Equus ferus, and/or other mammalian species. tPA is also used to refer to a class of drugs and/or agents known as thrombolytic agents. Commercially available thrombolytic agents and/or tPA brand names include: Activase; Cathflo Activase; Activase rt-PA; Alteplase; reteplase; tenecteplase; lanoteplase; Eminase; anistreplase; Retavase; Streptase; streptokinase; kabikinase; TNKase; Abbokinase, Kinlytic; Actilyse; Activacin; Aktylize; and rokinase.

In some aspects of the methods of the present disclosure, tPA can be replaced with any other thrombolytic agent that is known in the art. A thrombolytic agent is an agent (e.g. a drug, a small molecule, an antibody, a protein, a biologic, etc.) that can dissolve a clot and/or reopen an artery or vein. Thrombolytic agents can include, but are not limited to eminase (anistreplase), retavase (reteplase), streptase (streptokinase, kabikinase), t-PA (class of drugs that includes Activase), Alteplase, TNKase (tenecteplase), Abbokinase, Kinlytic (urokinase) or any other thrombolytic agent known in the art.

As used herein, the term “Individual” or “subject” or “mammal” means a human or non-human mammal selected for treatment or therapy.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Compositions

In some embodiments, without limitation, the methods described herein may utilize compositions and/or formulations containing mammalian cell-derived exosomes and/or microvesicles, in combination with tPA and/or thrombectomy. In various embodiments, the compositions of the present methods are administered separately. In other embodiments, an illustrative composition comprises mammalian cell derived exosomes and/or microvesicles and tPA in a single composition. In some embodiments, the mammalian exosomes include exosomes and/or microvesicles derived from an exosome producing cell. In some embodiments, the mammalian exosomes useful in the methods of the present disclosure include exosomes and/or microvesicles derived from stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes which produce exosomes and secrete exosomes and/or microvesicles. The foregoing cells can be obtained via primary cell culture, or through commercial vendors. For example, cerebral endothelial cells are commercially available from the American Type Culture Collection (ATCC), Manassas, Va., USA.

In various embodiments, the compositions comprising mammalian derived exosomes and/or microvesicles (collectively referred to as extracellular vesicles) include an extracellular vesicle, for example, an exosome or a microvesicle containing a microRNA selected from miR-19a, miR-21, and miR-146a. In some embodiments, compositions of the present disclosure may comprise: mammalian exosomes which contain one or more of miR-19a, miR-21, and miR-146a RNA, human cells that are operable to synthesize extracellular vesicles containing miR-19a, miR-21, and/or miR-146a microRNA, or particles containing miR-19a, miR-21, and/or miR-146a for example, liposomes, microparticles, nanoparticles, or other common vehicles for delivery of nucleic acids commonly known in the art.

In some embodiments, mammalian extracellular vesicles can include particles derived from living cells, for example mammalian cells. In some embodiments, mammalian cells include cells that are known to produce exosomes, and microvesicles, for example, stem cells, mesenchymal stromal cells, umbilical cord cells, endothelial cells, (for example, cerebral endothelial cells), epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes

A mammalian extracellular vesicle can be derived or isolated in a variety of ways. In some embodiments, an illustrative embodiment may include exosomes and/or microvesicles derived from: mammalian (for example, human): stem cells, mesenchymal stromal cells, umbilical cord cells, endothelial cells, (for example, cerebral endothelial cells), epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes which produce exosomes and secrete exosomes and/or microvesicles. In various embodiments, these mammalian derived extracellular vesicles contain at least one of miR-19a, miR-21, and miR-146a microRNA. An exemplary exosome isolation method can be adapted from Thery C. et al., Isolation and Characterization of Exosomes from Cell Culture Supernatants and Biological Fluids, (2006), Curr. Protoc. Cell Biol.; Chapter 3: Unit 3.22, the disclosure of which is incorporated herein by reference in its entirety. In some non-limiting embodiments, miR-19a, miR-21, or miR-146a RNA can be induced for expression into an extracellular vesicle, e.g., an exosome. Moreover, miR-19a, miR-21, or miR-146a microRNA can be obtained for use in a mammalian exosome by either overexpression of miR-19a, miR-21, or miR-146a microRNA, or direct transfection and/or transformation of a host cell which is operable to produce and release exosomes containing one or more of the aforementioned miRNA. For example, mammalian cells can be modified to engineer expression of miR-19a, miR-21, or miR-146a microRNA. Additionally, in some illustrative embodiments, mammalian cells can be transfected or transformed with nucleic acid vectors, introducing nucleic acids encoding miR-19a, miR-21, and/or miR-146a. An illustrative example of miR-19a, miR-21, or miR-146a RNA transfection includes, but is not limited to, obtaining pre-miRNA-19a, 21, and/or 146a; plating cells on a suitable cell culture dish at 50% confluence; transfecting the pre-miRNA using Lipofectamine (or any other suitable transfection agent); confirming transfection using quantitative-PCR; washing the cells twice with PBS; and extracting the miR-19a, miR-21, and/or miR-146a microRNA using conventional, commercially available techniques, such as the mirVana miRNA isolation kit with phenol (Thermo Fisher Scientific) (Hu et al., MicroRNAs 125a and 455 Repress Lipoprotein-Supported Steroidogenesis by Targeting Scavenger Receptor Class B Type I in Steroidogenic Cells, Mol Cell Biol. 2012 December; 32(24): 5035-5045, the disclosure of which is incorporated herein by reference in its entirety).

In some embodiments, mammalian cells operable to produce and secrete exosomes and/or microvesicles can be transfected with miR-19a, miR-21, and/or miR-146a microRNA using common techniques known to those with ordinary skill in the art, and/or by using commercially available kits (e.g., Exo-fect Exosome Transfection Kit, System Biosciences). Furthermore, cells can be reprogrammed to express mammalian exosomes and/or miR-19a, miR-21, and/or miR-146a microRNA. An exemplary microRNA reprogramming method is illustrated by Trivedi et al., “Modification of tumor cell exosome content by transfection with wt-p53 and microRNA-125b expressing plasmid DNA and its effect on macrophage polarization”, Oncogenesis. 2016 August; 5(8): e250, the disclosure of which is incorporated herein by reference in its entirety. In a non-limiting embodiment, a plasmid containing pre-miR-19a, pre-miR-21, and/or pre-miR-146a microRNA is isolated and purified. Next, hyaluronic acid-poly(ethylene imine) and hyaluronic acid (HA)-poly(ethylene glycol) (PEG) (HA-PEI/HA-PEG) blend nanoparticles are then obtained by combining 50 mg of maleimide-PEG-amine to 1-Ethyl-3-(3-dimethylaminopropyl)-carbodimide (EDC)/N-hydroxysuccinimide (NHS) activated HA, and dissolving the HA-PEI and HA-PEG solutions in PBS. Cells such as stem cells, mesenchymal stromal cells, umbilical cord cells, endothelial cells, cerebral endothelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, mastocytes, or any cell with an endomembrane system, can be plated and treated with a suitable amount of one or more plasmids containing miR-19a, miR-21, or miR-146a (e.g., 1-20 μg) encapsulated in the nanoparticles. Finally, exosomes can be isolated using techniques described above, by using commercially available kits, or by taking cell supernatant from, and centrifuging at 2000 g for 30 min to remove cell debris; taking the supernatant and adding it to a commercially available exosome isolation reagent, followed by incubation overnight at 4° C.; further centrifuged at 10,000 g for 1 hour at 4° C.; and aspiration of the supernatant followed by resuspending the exosome pellet in sterile PBS.

In some embodiments, cells can be induced to release and/or secrete an exosomes and/or microvesicles in response to a variety of signals including, but not limited to, cytokines, mitogens, and/or any other method of paracrine/autocrine signaling (see Saunderson et al., “Induction of Exosome Release in Primary B Cells Stimulated via CD40 and the IL-4 Receptor”, J Immunol. 2008 Jun. 15; 180(12):8146-52, the disclosure of which is incorporated herein by reference in its entirety).

In some embodiments, cells can be induced to release and/or secrete exosomes and/or microvesicles by modulating intracellular calcium (Ca²⁺) content. An exemplary illustrative technique for stimulating a mammalian exosome and/or a microvesicle containing miR-19a, miR-21, or miR-146a is provided by Savina et al., “Exosome release is regulated by a calcium-dependent mechanism in K562 cells”, the disclosure of which is incorporated herein by reference in its entirety. After selecting the suitable cell type, for example, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes and/or any cell with an endomembrane system, a compound that influences Na⁺/H⁺ exchange and/or intracellular calcium (Ca²⁺) content (e.g., an ionophore such a monesin), can be applied to stimulate mammalian exosome release. Subsequent to mammalian exosome stimulation, the exosomes and/or microvesicles can be isolated using any one of the techniques known to those with ordinary skill, and/or enumerated herein.

Some embodiments may call for mammalian extracellular vesicles, for example, exosomes to be produced by stimulating and/or inducing the overproduction of exosomes and/or microvesicles in either stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, mastocytes, or any one or more of the abovementioned cells, and/or any cell with an endomembrane system, that has been transformed or transfected to overexpress miR-19a, miR-21, or miR-146a microRNA, using techniques known to those with ordinary skill, and/or enumerated herein, for example as provided in: Amigorena S, Raposo G, Clayton A: “Isolation and characterization of exosomes from cell culture supernatants and biological fluids”. Curr. Protoc. Cell Biol. 2006 April; Chapter 3: Unit 3.22, the disclosure of which is incorporated herein by reference in its entirety. Typically, 100 mL of cultured media is used by pooling from multiple dishes. The media is centrifuged at 300×g for 10 min at 4° C. to remove any intact cells, followed by a 2,000×g spin for 20 min at 4° C. to remove dead cells and finally a 10,000×g spin for 30 min at 4° C. to remove cell debris. The media is then transferred to ultracentrifuge tubes and centrifuged at 100,000×g for at least 60 min at 4° C. in Optima TLX ultracentrifuge with 60 Ti rotor (Beckman Coulter, Mississauga, Canada). The supernatant containing exosome-free media is removed and the pellets containing exosomes plus proteins from media are resuspended in PBS. The suspension is centrifuged at 100,000×g for at least 60 min at 4° C. to collect final exosome pellets. The exosome pellet is then resuspended in an appropriate excipient or diluent in a desired volume to attain a specific concentration of exosomes per mL.

Exosomes may also be isolated using any of the techniques described by Willis et al., Toward Exosome-Based Therapeutics: Isolation, Heterogeneity, and Fit-for-Purpose Potency (2017) Front Cardiovasc Med. 4: 63, the disclosure of which is incorporated herein by reference in its entirety. Such isolation methods include Ultracentrifugation (i.e., 100,000-120,000×g); size-exclusion chromatography; commercially available isolation kits (e.g. ExoQuick and ExoELISA); and CD63 capture (exosome) ELISA, (Systems Biosciences, CA, USA).

An exemplary microvesicle isolation method can be adapted from R. Szatanek et al. Isolation of extracellular vesicles: Determining the correct approach (2015) Int J Mol Med. 2015 July; 36(1): 11-17, the disclosure of which is incorporated herein by reference in its entirety. Typically, for differential centrifugation/ultracentrifugation, intact cells, dead cells and cell debris are removed by centrifuging at 300×g for 10 min, 2,000×g for 10 min and 10,000×g for 30 min, respectively. Supernatant is transferred into a new test tube while the generated pellets are being discarded. After the 10,000×g spin, the supernatant is then subjected to a final ultracentrifugation at 100,000×g for 70 min, all centrifugation steps carried out at 4° C.

In some embodiments, the methods described herein can utilize compositions and/or formulations containing exosomes derived from a variety of exosome producing mammalian cells, for example, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes.

In various embodiments, mammalian cell derived exosomes include exosomes from mammalian cells which are operable to produce and secrete exosomes containing one or more of the following microRNAs: miR-19a, miR-21, and miR-146a microRNA. In some embodiments, compositions of the present disclosure comprise exosomes and/or microvesicles derived from cerebral endothelial cells (CECs). In closely related embodiments, compositions containing exosomes include compositions containing CEC derived exosomes in which at least a portion of exosomes contain one or more of the following microRNAs: miR-19a, miR-21, and miR-146a microRNA.

In some aspects, mammalian exosomes can be directly modified to increase the amount of miR-146a, miR-19a, miR-21 or any combination thereof present in the exosomes. In a non-limiting example, mammalian exosomes can be directly transfected with miR-146a, miR-19a, miR-21 or any combination thereof. In some aspects, mammalian exosomes can be loaded with miR-146a, miR-19a, miR-21 or any combination thereof using any exosome-loading technique known in the art.

An exemplary CEC isolation method can be adapted from Ruck et al., Isolation of Primary Murine Brain Microvascular Endothelial Cells, J Vis Exp. 2014; (93): 52204, the disclosure of which is incorporated herein by reference in its entirety. Alternatively, CECs may be obtained using the commercially available Microvascular Endothelial Cell Growth Kit-BBE (ATCC® PCS-110-040™), or the PrimaCell™, Rat Cerebral Venous Vascular Endothelial Cell Culture Kit (CHI Scientific).

In some embodiments, the compositions of the present disclosure may also comprise Tissue Plasminogen Activator (tPA) either in admixture with extracellular vesicles (exosomes and/or microvesicles), or in a separate composition for administration to a stroke subject in need thereof. tPA can be derived or isolated in a variety of ways. In some non-limiting embodiments, tPA can be obtained for use by either overexpression of tPA in an in vitro cell culture system, or direct transfection and/or transformation of a host cell. For example, mammalian cells can be modified to engineer expression of tPA, which can then be isolated and purified using methods known to those having ordinary skill in the art. Additionally, in some illustrative embodiments, mammalian cells can be transfected or transformed with nucleic acid vectors, introducing nucleic acids encoding tPA, including nucleic acids encoding tPA derived from species with a shared tPA homology, including, but not limited to, Homo sapiens, Bos Taurus, Mus musculus, Pan troglodytes, Sus scrofa, Gallus gallus, Equus ferus, and/or other mammalian species. An exemplary tPA transfection and isolation method can be adapted from Keyt B A et al., “A faster-acting and more potent form of tissue plasminogen activator,” Proc Natl Acad Sci USA. 1994 Apr. 26; 91(9):3670-4, the disclosure of which is incorporated herein by reference in its entirety. Briefly, Chines Hamster Ovary (CHO) cells are stably transfected with plasmids containing the tPA gene; the cells are then expanded in the presence methotrexate, and cultured in serum-free medium for 6 days. Conditioned cell culture medium is then concentrated and diafiltered, and tPA is isolated and purified via lysine affinity chromatography; tPA quantification can be performed using dual monoclonal assay sensitive to epitopes in the kringle 2 and the protease domains. Alternatively, tPA may be obtained by obtaining pre-mRNA-tPA; plating cells on a suitable cell culture dish at 50% confluence; transfecting the pre-mRNA using Lipofectamine (or any other suitable transfection agent); confirming transfection using quantitative-PCR; washing the cells twice with PBS; resuspending the cells with a cell scraper or with trypsin; centrifuging the cells, and resuspending the pellet in a lysis buffer or permeabilization buffer; centrifuging the suspension again; collecting the supernatant; and extracting the tPA using conventional, commercially available techniques, such as the ReadyPrep™ Protein Extraction Kit (Total Protein) (BioRad), or the Cell Lysis (Total Protein Extraction) kit (ThermoFisher).

In some embodiments, compositions comprising tPA include human tPA provided in natural or recombinant form and are commercially available as alteplase (Activase). In various embodiments, the composition comprising tPA may contain tPA at concentrations ranging from about 200 mg/mL to about 0.001 mg/mL, or from about 100 mg/mL to about 0.01 mg/mL.

In various embodiments, compositions of the present disclosure may include a composition comprising mammalian exosomes containing or enriched with miRNAs miRNA-19a, miRNA-21, and miRNA-146a. In various embodiments, the compositions of the present disclosure may include a composition comprising mammalian exosomes containing or enriched with miRNAs miRNA-19a, miRNA-21, and miRNA-146a and at least one pharmaceutically acceptable carrier, excipient, and diluent, for example, a pharmaceutically acceptable carrier, excipient, and/or diluent disclosed herein, or in Remington's Pharmaceutical Sciences (17th Ed., Mack Pub. Co. 1985), (the disclosure of which is incorporated herein by reference in its entirety) that is specifically used for administration of cells to human subjects, for example, a composition containing cells or subparts thereof dosed intravenously, or intra arterially, and which offers a reasonable benefit to risk ratio, or does not unduly cause the subject irritation or is incompatible with the cells or exosomes being administered to the subject, for example, a human subject.

In one embodiment, compositions of the present disclosure may include a composition comprising a modified population of cells, wherein the cells overexpress miR-146a over the level of expression of said miR-146a in control cells. In some embodiments, the cells are specifically enriched with miRNA-146a as compared to a control. In some embodiments, the cells have been modified through transient transfection with an miRNA-146a mimic. In other embodiments, the control cells are naïve cells that have not been transfected with an miRNA-146a mimic, or cells that have been transfected with a mimic control that does not express miRNA-146a. In yet other embodiments, the cells are human endothelial cells, or human endothelial cell progenitor cells.

In one embodiment, compositions of the present disclosure may include a composition comprising a population of mammalian exosomes enriched with miRNA-146a over the level of said miR-146a expression in control exosomes. In some embodiments, the exosomes are derived from human endothelial cells, or human endothelial cell progenitor cells that have been transfected with an miR-146a mimic.

In one embodiment, provided is a composition comprising a modified population of cells, wherein the cells overexpress miR-146a over the level of expression of said miRNA-146a in naïve or control cells. In another embodiment, the cells have been modified through transfection with an miRNA-146a mimic. In another embodiment, the transfection is transient or, in another embodiment, the transfection is stable. In another embodiment, the control cells do not express miRNA-146a. In some embodiments, the cells are human endothelial cells, or human endothelial cell progenitor cells.

In one embodiment, the cells provided herein overexpress miR-146a by at least 2, 3, 4, 5, 6, 7, 8, 9, or 10 fold over the level of expression of said miRNA-146a in naïve or control cells. In another embodiment, the cells provided herein overexpress miR-146a by at least 10-20, 21-30, 41-50, 51-60, 61-70, 71-80, 81-90, or 91-100 fold over the level of expression of said miRNA-146a in naïve or control cells. In another embodiment, the cells provided herein overexpress miR-146a by at least 10, 20, 30, 40 or 50 fold over the level of expression of said miRNA-146a in naïve or control cells.

In one embodiment, the cells provided herein overexpress miR-146a by at least 10 to 50% over the level of expression of said miRNA-146a in naïve or control cells. In one embodiment, the cells provided herein overexpress miR-146a by at least 2, 3, 4, 5, 6, 7, 8, 9, or 10 percent over the level of expression of said miRNA-146a in naïve or control cells. In another embodiment, the cells provided herein overexpress miR-146a by at least 10-20, 21-30, 41-50, 51-60, 61-70, 71-80, 81-90, or 91-100 percent over the level of expression of said miRNA-146a in naïve or control cells. In another embodiment, the cells provided herein overexpress miR-146a by at least 10, 20, 30, 40 or 50 percent over the level of expression of said miRNA-146a in naïve or control cells.

In one embodiment, provided is a composition comprising a population of mammalian exosomes enriched with miR-146a over the level of said miRNA-146a expression in naïve or control exosomes. In one embodiment, the exosomes are derived from human endothelial cells, or human endothelial cell progenitor cells that have been transfected with an miRNA-146a mimic. In other embodiments, the cells are cerebral endothelial cells or mesenchymal stromal cells.

In one embodiment, provided is a composition comprising mammalian exosomes enriched with at least one miRNAs selected from the group consisting of: miRNA-19a, miRNA-21, and miRNA-146a. In one embodiment, the miRNA-146a is selectively overexpressed in the mammalian exosomes over the level of miRNA-146a expression in naïve or control exosomes.

In one embodiment, the mammalian exosomes provided herein overexpress miR-146a by at least 2 to 10 fold over the level of said miRNA-146a expression in naïve or control exosomes. In one embodiment, the mammalian exosomes provided herein overexpress miR-146a by at least 2, 3, 4, 5, 6, 7, 8, 9, or 10 fold over the level of expression of said miRNA-146a in naïve or control exosomes. In another embodiment, the mammalian exosomes provided herein overexpress miR-146a by at least 10-20, 21-30, 41-50, 51-60, 61-70, 71-80, 81-90, or 91-100 fold over the level of expression of said miRNA-146a in naïve or control exosomes. In another embodiment, the exosomes provided herein overexpress miR-146a by at least 10, 20, 30, 40 or 50 fold over the level of expression of said miRNA-146a in naïve or control exosomes.

In one embodiment, the mammalian exosomes overexpress miR-146a by at least 10 to 50% over the level of the miRNA-146a expression in naïve or control exosomes. In another embodiment, the exosomes provided herein overexpress miR-146a by at least 10 to 50% over the level of expression of said miRNA-146a in naïve or control exosomes. In one embodiment, the exosomes provided herein overexpress miR-146a by at least 2, 3, 4, 5, 6, 7, 8, 9, or 10 percent over the level of expression of said miRNA-146a in naïve or control exosomes. In another embodiment, the exosomes provided herein overexpress miR-146a by at least 10-20, 21-30, 41-50, 51-60, 61-70, 71-80, 81-90, or 91-100 percent over the level of expression of said miRNA-146a in naïve or control exosomes. In another embodiment, the exosomes provided herein overexpress miR-146a by at least 10, 20, 30, 40 or 50 percent over the level of expression of said miRNA-146a in naïve or control exosomes.

In related embodiments, the composition may include human exosomes derived from a human cell culture. Human cell cultures can include, a mammalian cell selected from stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, which in each case, the mammalian cell is operable to produce exosomes containing or enriched with miRNAs miRNA-19a, miRNA-21, and miRNA-146a. In some embodiments, the mammalian cell includes: human endothelial cells, or human endothelial cell progenitor cells, each of which are operable to produce an exosome containing or enriched with miRNAs miRNA-19a, miRNA-21, and miRNA-146a. In some embodiments the mammalian cell is a tissue cultured cell which produce exosomes containing or enriched with miRNAs miRNA-19a, miRNA-21, and miRNA-146a, for example, human endothelial cells, or human endothelial cell progenitor cells, such as cerebral endothelial cells (CEC) containing or enriched with miRNAs miRNA-19a, miRNA-21, and miRNA-146a.

Formulations

Methods for preparing a formulation of mammalian exosomes are known, and/or are readily apparent to those skilled in the art. An exemplary formulation method can be adapted from Remington's Pharmaceutical Sciences (17th Ed., Mack Pub. Co. 1985); Remington: Essentials of Pharmaceutics (Pharmaceutical Press, 2012), the disclosure of which is incorporated herein by reference in its entirety. Methods for formulating a nucleic acid, for example, miR-19a, miR-21, or miR-146a microRNA and a pharmaceutically acceptable vehicle, carrier, or excipient for the delivery of nucleic acids are provided in U.S. Patent Application Publication No. US 2013/0017223A1, Ser. No. 13/516,335, filed on Dec. 17, 2016, the disclosure of which is incorporated herein by reference in its entirety. Methods for formulating a pharmaceutically acceptable vehicle, carrier, or excipient for the delivery of miRNA are provided in U.S. Pat. No. 9,301,969B2, Ser. No. 13/822,641, filed on Sep. 9, 2011, the disclosure of which is incorporated herein by reference in its entirety. Methods for preparing a formulation of exosomes containing an agent are provided in U.S. Patent Application Publication No. US 2013/0156801A1, Ser. No. 13/327,244, filed on Dec. 15, 2011, the disclosure of which is incorporated herein by reference in its entirety. Furthermore, methods for preparing formulations for the exosome mediated delivery of biotherapeutics are provided in World Intellectual Property Organization Patent Application Publication No. WO 2013/084000 A2, filed on Dec. 7, 2012; and U.S. Patent Application Publication No. US 2016/0346334 A1, Ser. No. 15/116,579, filed on Feb. 5, 2015, the disclosures of these patent references are incorporated herein by reference in their entirety.

In some embodiments, without limitation, the methods described herein can utilize formulations containing one or more isolated mammalian exosomes that are contained within a pharmaceutically acceptable vehicle, carrier, adjuvants, additives and/or excipient that allows for storage and handling of the agents before and during administration. Moreover, in accordance with certain aspects of the present disclosure, the agents suitable for administration may be provided in a pharmaceutically acceptable vehicle, carrier, or excipient with or without an inert diluent. Further, in addition to the above-described components, the formulation may contain additional lubricants, emulsifiers, suspending-agents, preservatives, or the like. Accordingly, the pharmaceutically acceptable vehicle, carrier, adjuvants, additives and/or excipient must be acceptable in the sense of being compatible with the other ingredients of the formulation and not deleterious to the recipient thereof, i.e., are sterile compositions and contain pharmaceutically acceptable vehicle, carrier, adjuvants, additives that are approved by the US Food and Drug Administration (FDA) for administration to a human subject.

Formulations containing mammalian exosomes and/or microvesicles may be prepared with one or more carriers, excipients, and diluents. Exemplary carriers, excipients and diluents can include one or more of sterile saline, phosphate buffers, Ringer's solution, and/or other physiological solutions that are used in the preparation of cellular therapies for administration in humans. An exemplary method for generating formulations containing mammalian exosomes is illustrated by Haqqani et al., “Method for isolation and molecular characterization of extracellular microvesicles released from brain endothelial cells,” Fluids Barriers CNS. 2013 Jan. 10; 10(1):4, the disclosures of which are incorporated herein by reference in its entirety. An alternative method for generating formulations containing mammalian exosomes is illustrated by Li et al., “Exosomes Derived From Human Umbilical Cord Mesenchymal Stem Cells Alleviate Liver Fibrosis”. Stem Cells Dev. 2013; 22:845-854, and Qiao et al., “Human mesenchymal stem cells isolated from the umbilical cord”, Cell Biol Int. 2008 January; 32(1):8-15. Epub 2007 Aug. 19, the disclosures of which are incorporated herein by reference in its entirety.

In certain embodiments, formulations comprising one or more mammalian exosomes can contain further additives including, but not limited to, pH-adjusting additives, osmolarity adjusters, tonicity adjusters, anti-oxidants, reducing agents, and preservatives. Useful pH-adjusting agents include acids, such as hydrochloric acid, bases or buffers, such as sodium lactate, sodium acetate, sodium phosphate, sodium citrate, sodium borate, or sodium gluconate. Further, the compositions of the invention can contain microbial preservatives. Useful microbial preservatives include methylparaben, propylparaben, and benzyl alcohol. The microbial preservative is typically employed when the formulation is placed in a vial designed for multidose use. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., .alpha.-tocopherol (vitamin E)), preservatives, chelating agents (e.g., EDTA and/or EGTA), viscomodulators, tonicifiers (e.g., a sugar such as sucrose, lactose, and/or mannitol), flavorants, colorants, odorants, opacifiers, suspending agents, binders, fillers, plasticizers, lubricants, and mixtures thereof. The amounts of such additives can be readily determined by one skilled in the art, according to the particular properties desired. Further, the formulation may comprise different types of carriers suitable for liquid, solid, or aerosol delivery.

In certain embodiments, a formulation can be made by suspending mammalian exosomes and/or microvesicles in a physiological buffer with physiological pH, for example, a sterile buffer solution such as phosphate buffer solution (PBS); sterile 0.85% NaCl solution in water; or 0.9% NaCl solution in Phosphate buffer having KCl. Physiological buffers (i.e., a 1×PBS buffer) can be prepared, for example, by mixing 8 g of NaCl; 0.2 g of KCl; 1.44 g of Na₂HPO₄; 0.24 g of KH₂PO₄; then, adjusting the pH to 7.4 with HCl; adjusting the volume to 1 L with additional distilled H₂O; and sterilizing by autoclaving.

In some embodiments, methods for the treatment of stroke or stroke symptoms may include administration of a formulation containing mammalian exosomes to be combined with a biological fluid such as blood, nasal secretions, saliva, urine, breast milk, cerebrospinal fluid, and/or any other natural matrix that represents a minimalist processing step (i.e., a step/storage component that reduces the possibility of influencing mammalian exosome surface characteristics and/or behavior/integrity upon introduction to the subject/patient); an exemplary illustrative technique for formulating mammalian exosomes and/or a microvesicle, with one of the aforementioned biofluids, is provided by Witwer et al., Standardization of sample collection, isolation and analysis methods in extracellular vesicle research, J Extracell Vesicles. 2013; 2, the disclosure of which is incorporated herein by reference in its entirety.

In some embodiments, the potency/quantity of a formulation containing mammalian exosomes and/or microvesicles can be quantified using conventional tools and techniques known to those having ordinary skill in the art, e.g., the electrical resistance nano pulse method, using commercially available tools and components, to determine the yield of an exosome preparation (e.g., qNano; IZON Science Ltd., Oxford, UK) (see Komaki et al., Exosomes of human placenta-derived mesenchymal stem cells stimulate angiogenesis, Stem Cell Res Ther. 2017; 8: 219, the disclosure of which is incorporated herein by reference in its entirety. Furthermore, the dosage of a mammalian exosome, and/or the miR-19a, miR-21, or miR-146a microRNA contents contained therein, may also be confirmed/quantified using the tools available to one having ordinary skill such as tunable resistive pulse sensing, protein quantification (e.g., Protein Assay Rapid Kit, Wako Pure Chemicals, Osaka, Japan), nanoparticle tracking analysis, enzyme-linked immunosorbent assay (ELISA), flow cytometry, dynamic light scattering, cell equivalents, fingerprinting (i.e., quantifying surrogate markers as an indication), and/or using a sample to elicit a response on an in vitro/in vivo surrogate (see Willis et al., Toward Exosome-Based Therapeutics: Isolation, Heterogeneity, and Fit-for-Purpose Potency, Front Cardiovasc Med. 2017; 4: 63, the disclosure of which is incorporated herein by reference in its entirety).

Prior to administration, in some embodiments, depending on the quantity and/or content of the mammalian exosomes-will require appropriate storage and/or handling, the process and/or conditions of which should be dictated by the said quality/content of the mammalian exosomes, and good medical practice. For example, in some non-limiting embodiments, a mammalian exosome formulation comprising exosomes, for example, CEC derived exosomes; or pharmaceutically acceptable compositions containing CEC derived exosomes described herein, with any one of the abovementioned carriers, excipients, and diluents, may be stored at −20° C., for a length of time that will not degrade the mammalian exosomes. Storage formulations that have been successful include buffers that resist pH shifts during freezing/thawing, and are devoid of glycerol and/or dimethyl sulfoxide (see Willis et al., Toward Exosome-Based Therapeutics: Isolation, Heterogeneity, and Fit-for-Purpose Potency, Front Cardiovasc Med. 2017; 4: 63, the disclosure of which is incorporated herein by reference in its entirety). Furthermore, in some non-limiting embodiments, the container should be tailored to the mammalian exosomes, and should consist of a material that supports mammalian exosome storage (e.g., cell culture/clinical grade glassware or plastic) (see Lener et al., Applying extracellular vesicles based therapeutics in clinical trials, J Extracell Vesicles. 2015; 4: 10.3402/jev.v4.30087, the disclosure of which is incorporated herein by reference in its entirety).

When necessary, proper fluidity of the compositions and formulations described herein can be maintained, for example, by the use of a coating such as lecithin, by the maintenance of the required size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for such compositions of mammalian exosomes. Furthermore, various additives which enhance the stability, sterility, and/or isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. Prolonged absorption of the injectable pharmaceutical form can be brought about by the use of agents delaying absorption, for example, aluminum monostearate and gelatin. According to some embodiments of the present disclosure, however, any vehicle, diluent, or additive used would have to be compatible with mammalian exosomes.

Sterile injectable solutions can be prepared by incorporating mammalian exosomes and/or microvesicles utilized in practicing some embodiments of the present disclosure in the required amount of the appropriate solvent with various other ingredients, as desired.

In some non-limiting embodiments, a formulation can be prepared by combining mammalian exosomes and/or microvesicles isolated from stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes. In some embodiments, the mammalian cell derived exosomes and/or microvesicles contain one or more of: miR-19a, miR-21, or miR-146a microRNA. In some illustrative embodiments, a formulation may comprise one or more of CEC derived exosomes and/or microvesicles; and a pharmaceutically acceptable carrier, excipient, or diluent. In some embodiments, a formulation containing a mammalian exosome can include a composition comprising CEC derived exosomes and/or microvesicles containing one or more of miR-19a, miR-21, and miR-146a microRNA described herein, in addition to any one or more of the abovementioned carriers, excipients, and diluents.

Formulations containing mammalian exosomes and tPA may be prepared with one or more carriers, excipients, and diluents. Exemplary carriers, excipients and diluents can include one or more of sterile saline, phosphate buffers, Ringer's solution, and/or other physiological solutions that are used in the preparation of cellular therapies for administration in humans. Alternatively, in some embodiments, tPA may be formulated separately from mammalian exosomes. For example, tPA may be supplied as lyophilized form, to be resuspended and diluted to the final effective dose in 0.9% Sodium Chloride solution, or 5% Dextrose Injection solution. Lyophilized tPA is commercially available in various amounts, for example, in 50 mg vials, and 100 mg vials, marketed as Activase® (Alteplase) (Genentech, Inc.).

In some embodiments, a formulation of tPA can be obtained from commercially available sources, for example, Activase® (Alteplase) (Genentech, Inc.); Retevase® (reteplase) (FA Davis); TNKase® (tenecteplase) (Genentech); Streptase® (streptokinase) (Sanofi Aventis); or Eminase @(anistreplase).

Administration

As used herein, the term “administering” means providing an agent to a subject in need thereof, and includes, but is not limited to, administering by a medical professional and self-administering. In some embodiments, without limitation, the methods described herein can be administered intravenously; intraarterially; subcutaneously; intramuscularly; intraperitoneally; stereotactically; intranasally; mucosally; intravitreally; intrastriatally; or intrathecally. The foregoing administration routes can be accomplished via implantable microbead (e.g., microspheres, sol-gel, hydrogels); injection; continuous infusion; localized perfusion; catheter; or by lavage. In some embodiments, the compositions and formulations of the present disclosure are administered via injection or infusion, preferably by intravenous, subcutaneous, or intraarterial administration. Methods for administering a formulation of a mammalian exosome and/or tPA can adapted from Remington's Pharmaceutical Sciences (17th Ed., Mack Pub. Co. 1985), the disclosure of which is incorporated herein by reference in its entirety.

In various embodiments, methods are provided for the prevention and/or treatment of a cerebrovascular injury in a patient who has suffered a stroke, comprising administering to the subject in need thereof, a therapeutically effective amount of a combination of mammalian exosomes and tPA, a therapeutically effective amount of a combination of mammalian exosomes and a thrombectomy procedure, or a therapeutically effective amount of a combination of mammalian exosomes, tPA, and a thrombectomy procedure. The methods contemplate administering one or more compositions that are pharmaceutically acceptable for the treatment of humans, particularly humans who have suffered a stroke and are deemed safe and effective. In various embodiments, the administration of the mammalian exosomes, tPA, and the performance of the thrombectomy procedure can be accomplished using an administration method known to those of ordinary skill in the art.

Dosages for a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the mammalian exosome employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

“Dosage unit” means a form in which a pharmaceutical agent or agents are provided, e.g. a solution or other dosage unit known in the art. Further, as used herein, “Dose” means a specified quantity of a pharmaceutical agent provided in a single administration, or in a specified time period. In certain embodiments, a dose can be administered in one, two, or more, boluses, infusions, or injections. For example, in certain embodiments where intravenous or subcutaneous administration is desired, the desired dose may require a volume not easily accommodated by a single injection, therefore, two or more injections can be used to achieve the desired dose, or one or more infusions are administered. In certain embodiments, the pharmaceutical agent is administered by infusion over an extended period of time or continuously. Doses can be stated as the amount of pharmaceutical agent per hour, day, week, or month. Doses can be expressed as μg/kg, mg/kg, g/kg, mg/m² of surface area of the patient, or number of exosomes.

Therapeutic compositions comprising mammalian exosomes are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the EC₅₀ of the relevant formulation, and/or observation of any side-effects of the mammalian exosomes and/or tPA at various concentrations, e.g., as applied to the mass and overall health of the patient. Administration can be accomplished via single or divided doses. Various factors may be used by a skilled practitioner, for example, a clinician, physician, or medical specialist to properly administer mammalian exosomes, tPA, and/or perform the thrombectomy procedure. For example, if using a composition containing both mammalian exosomes and tPA that can circulate freely in the bloodstream, the composition or formulation of the combination may be administered intravenously, subcutaneously or intra-arterially. Similarly, separate compositions, each containing either the mammalian exosomes or tPA, each can be administered intravenously, subcutaneously or intra-arterially. In some embodiments, the mammalian exosomes and/or microvesicles may be administered prior to, concomitantly with or subsequent to the administration of the tPA. In some embodiments, the mammalian exosomes and/or microvesicles are administered prior to the administration of the tPA. In related embodiments, a first dose of mammalian exosomes is administered as an intravenous bolus, followed by the administration of tPA, which may be administered as an infusion. The mammalian exosomes and tPA can be administered in various ways; for example, mammalian exosomes can be administered alone, or as an active ingredient in combination with pharmaceutically acceptable carriers, diluents, adjuvants and vehicles, or in concert with tPA. The mammalian exosomes can be administered parenterally, for example, intravenously, intra-arterially, subcutaneously administration as well as intrathecal and infusion techniques, or by local administration or direct administration (stereotactic administration) to the site of disease or pathological condition. Repetitive administrations of the mammalian exosomes, and/or tPA, may also be useful, where short term or long term (for example, hours, days or weeklong administration is desirable). In various embodiments, tPA may be administered parenterally, preferably by intravenous administration either by direct injection, infusion or via catheter administration as approved for the treatment of acute ischemic stroke by regulatory review by a competent regulatory body, for example, the US Food and Drug Administration (FDA) or the European Medicines Agency.

The subject or patient being treated is a warm-blooded animal and, in particular, mammals, including humans. The pharmaceutically acceptable carriers, diluents, adjuvants and vehicles as well as implant carriers generally refer to inert, non-toxic solid or liquid fillers, diluents or encapsulating material not reacting with the active components of the invention. In some embodiments, mammalian exosomes may be altered by use of antibodies to cell surface proteins to specifically target tissues of interest.

“Mammal” or “mammalian” refers to a human or non-human mammal, including, but not limited to, mice, rats, rabbits, dogs, cats, pigs, and non-human primates, including, but not limited to, monkeys and chimpanzees.

In some embodiments, when administering mammalian exosomes parenterally, it will generally be formulated in a unit dosage injectable form (for example, in the form of a liquid, for example, a solution, a suspension, or an emulsion). Some pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

A pharmacological formulation of some embodiments may be administered to the patient in an injectable formulation containing any compatible carrier, such as various vehicle, adjuvants, additives, and diluents; or the inhibitor(s) utilized in some embodiments may be administered parenterally to the patient in the form of slow-release subcutaneous implants or vectored delivery, iontophoretic, polymer matrices, liposomes, and microspheres. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the mammalian exosomes and/or tPA. In addition, a pump-based hardware delivery system may be used to deliver one or more embodiments.

Examples of systems in which release occurs in bursts includes, e.g., systems in which the mammalian exosome cargo is entrapped in liposomes which are encapsulated in a polymer matrix, the liposomes being sensitive to specific stimuli, e.g., temperature, pH, light or a degrading Many other such implants, delivery systems, and modules are well known to those skilled in the art.

In some embodiments, without limitation, mammalian exosomes may be administered initially by an infusion or intravenous injection to bring blood levels of one or more of miR-19a, miR-21, or miR-146a microRNA to a suitable level. The patient's levels are then maintained by an intravenous dosage form of mammalian exosomes, although other forms of administration, dependent upon the patient's condition and as indicated above, can be used. The quantity to be administered and timing of administration may vary for the patient being treated.

Additionally, in some embodiments, without limitation, a mammalian exosome may be administered in situ to bring internal levels to a suitable level. The patient's levels are then maintained as appropriate in accordance with good medical practice by appropriate forms of administration, dependent upon the patient's condition. The quantity to be administered and timing of administration may vary for the patient being treated.

In certain non-limiting embodiments, mammalian exosomes are administered via intravenous injection, for example, a subject is injected intravenously with a formulation of mammalian exosomes suspended in a suitable carrier using a needle with a gauge ranging from about 7-gauge to 25-gauge (see Banga (2015) Therapeutic Peptides and Proteins: Formulation, Processing, and Delivery Systems; CRC Press, Boca Raton, Fla.). An illustrative example of intravenously mammalian exosomes includes, but is not limited to, uncovering the injection site; determining a suitable vein for injection; applying a tourniquet and waiting for the vein to swell; disinfecting the skin; pulling the skin taut in the longitudinal direction to stabilize the vein; inserting needle at an angle of about 35 degrees; puncturing the skin, and advancing the needle into the vein at a depth suitable for the subject and/or location of the vein; holding the injection means (e.g., syringe) steady; aspirating slightly; loosening the tourniquet; slowly injecting the mammalian exosomes; checking for pain, swelling, and/or hematoma; withdrawing the injection means; and applying sterile cotton wool onto the opening, and securing the cotton wool with adhesive tape.

In some embodiments, the initial administration may include an infusion of mammalian exosomes via intravenous administration over a period of 1 minute to 120 minutes. Subsequent doses of the mammalian exosomes can be accomplished using intravenous injections or by infusion. Each dose administered may be therapeutically effective doses or suboptimal doses repeated if needed.

Any appropriate routes of exosome and/or microvesicle administration known to those of ordinary skill in the art may comprise embodiments of the invention. In some embodiments, isolated mammalian exosomes and/or microvesicles contained within a pharmaceutically acceptable vehicle, carrier, or excipient, or miR-19a, miR-21, or miR-146a microRNA containing agents derived from mammalian cells, for example, stem cells, mesenchymal stromal cells, umbilical cord cells, endothelial cells, cerebral endothelial cells, epithelial cells Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, or their internal components thereof, can be administered and dosed in accordance with good medical practice, taking into account the clinical condition of the individual patient, the site and method of administration, scheduling of administration, patient age, sex, body weight and other factors known to medical practitioners.

In some embodiments, the administration is designed to supply the mammalian exosomes and/or microvesicles and tPA to the tissue that requires the effects provided by the mammalian exosomes and the tPA to prevent or treat the stroke and/or stroke symptoms and/or cerebrovascular injury. In some embodiments, the target tissue includes one or more of: the blood vessels of the subject, the blood vessels of the brain and brain tissue.

For example, in one embodiment, a dose of the mammalian exosomes and/or microvesicles may include administration of about 1×10⁷ to about 1×10¹⁷ exosomes administered per dose, one or more times per day, or one or more times per week, or one or more times per month. In some embodiments, when the mammalian exosomes and tPA are dosed separately, a dosage unit of exosomes and/or microvesicles may include a container for example, a vial containing 10⁷ to 10¹⁷ exosomes and/or 10⁷ to 10¹⁷ microvesicles. In certain embodiments, a dosage unit of exosomes and/or microvesicles is a vial containing 10⁷ to 10¹⁷ exosomes and/or 10⁷ to 10¹⁷ microvesicles and at least one pharmaceutically acceptable excipient. For tPA administration, the dosage of tPA may include 500 μg to about 500 mg, or from about 750 μg to about 300 mg, or from about 1 mg to about 200 mg, or from about 10 mg to about 150 mg administered per dose or total dose, wherein the total dose may be divided doses, the first dose administered as an initial bolus and the remainder infused over a period of time ranging from about 5 minutes to about 120 minutes. In some embodiments, tPA may be formulated as a 0.9 mg/kg solution, and may be administered intravenously, wherein the tPA administered is not to exceed 90 mg total dose; and wherein the tPA is administered 10% of the total dose as an initial IV bolus over 1 minute and the remainder infused over 60 minutes. In this example, the mammalian exosomes and/or microvesicles, for example mammalian exosomes and/or microvesicles, for example, mammalian exosomes and/or microvesicles, such as an exosome and/or microvesicle derived from one or more cells selected from: stem cells, mesenchymal stromal cells, umbilical cord cells, endothelial cells, cerebral endothelial cells, epithelial cells Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, or their internal components thereof, or for example, any of the foregoing mammalian cell derived exosomes and/or microvesicles containing one or more of: miR-19a, miR-21, and miR-146a microRNA, is administered prior to, concomitantly with or subsequent to the administration of tPA. In some embodiments, the mammalian exosomes and/or microvesicles are dosed before the administration of the tPA or concomitantly with the tPA and is then administered one or more times after the administration of the tPA, for example, one or more doses dosed daily, one or more times per day, one or more times per week or one or more times per month for one week to 12 months after the initial stroke.

In another embodiment, the mammalian exosomes and/or microvesicles, for example, an exosome and/or microvesicle derived from one or more mammalian cells selected from: stem cells, mesenchymal stromal cells, umbilical cord cells, endothelial cells, cerebral endothelial cells, epithelial cells Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, or their internal components thereof, or for example, any of the foregoing mammalian cell derived exosomes and/or microvesicles containing one or more of: miR-19a, miR-21, and miR-146a microRNA, is administered prior to, and/or concomitantly with and/or subsequent to the performance of thrombectomy. In some embodiments, the mammalian exosomes and/or microvesicles described herein are dosed before the performance of the thrombectomy procedure. In some embodiments, the mammalian exosomes and/or microvesicles described herein are dosed concomitantly with the thrombectomy procedure. In some embodiments, the mammalian exosomes and/or microvesicles described herein are dosed before the performance of the thrombectomy procedure and is then administered one or more times after the thrombectomy procedure, for example, one or more doses dosed hourly, or one or more times per day, or one or more times per week, or one or more times per month for one week to 12 months after the initial stroke. In some related embodiments, tPA may also be dosed prior to, or concomitantly with the thrombectomy procedure.

Methods of Treatment

The inventors have unexpectedly found that when a subject experiences an ischemic stroke, for example, an acute ischemic stroke, the administration of a therapeutically effective amount of a combination comprising mammalian exosomes and tPA provides one or more unexpected therapeutic benefits: (1) increases proteolysis of fibrin in a thrombus; (2) extends the therapeutic window for tPA treatment beyond 3-4.5 hours; (3) reduces the size of a thrombus by at least 10%-50%, for example, at least 30%; (4) increases the rate and extent of vessel recanalization; (5) increases microvascular reperfusion without increased brain hemorrhage; (6) reduces leakage of the blood-brain-barrier; (7) reduces adhesion molecules, (8) reduces vascular inflammation, (9) reduces procoagulant and/or prothrombotic conditions and (10) attenuates infarct expansion, when compared to administration of tPA alone.

Without wishing to be bound by theory, mammalian exosomes can exert their therapeutic effect on cerebral endothelial cells to reduce vascular injury and formation of secondary thrombosis via delivering an exosome and/or microvesicle cargo comprising one or more of miR-19a, -21, and miR-146a microRNA to repress the network of microRNAs/proteins that promote vascular injury and thrombogenicity.

In one embodiment, exosomes that are enriched with miRNA-146a protect cellular tight junction integrity. In another embodiment, exosomes having overexpressed miRNA-146a protect the blood brain barrier from leakage. In another embodiment, exosomes enriched with miRNA-146a prevent the blood brain barrier leakage. In another embodiment, exosomes enriched with miRNA-146a reduce blood brain barrier leakage. In another embodiment, exosomes enriched with miRNA-146a eliminate blood brain barrier leakage. In another embodiment, exosomes enriched with miRNA-146a treat blood brain barrier leakage when administered to a subject in need thereof.

Accordingly, the present disclosure has identified several unexpected findings as illustrated in the examples section below. One such unexpected finding includes the discovery that when compared to tPA alone, intravenous (IV) administration of mammalian exosomes in combination with tPA, administered more than double the equivalent acceptable time period for tPA therapy in humans, significantly reduced ischemic lesion size and improved neurological outcome by facilitating recanalization and augmenting microvascular reperfusion without increasing brain hemorrhage. Another example of the unexpected findings disclosed herein, is that exosomes derived from the patient brain clot which caused the stroke, induces dysfunction of healthy CECs, suggesting that brain blood clot-exosomes communicate with cerebral endothelial cells and trigger endothelial cells to induce proteins that promote stability of the thrombi, secondary thrombosis in down-stream microvessels, and blood brain barrier (BBB) impairment. Furthermore, the administration of healthy mammalian exosomes (for example, those derived from CECs) diminished clot-exosome-upregulated proteins and BBB leakage.

As the examples below demonstrate, mammalian exosomes target cerebral endothelial cells to reduce vascular injury, leading to suppression of secondary thrombosis via suppression of the network of miRNAs/proteins that promote vascular injury and thrombogenicity and suppress BBB leakage when used in combination with tPA. In addition, the therapeutic combination of mammalian exosomes and tPA provides significant benefits, for example: significantly reduces infarct volume; leads to improvement in neurological outcomes; promotes recanalization; enhances microvascular patency and integrity; reduces ischemic brain damage; elevates miR-21 and miR-146a expression; and reduces proteins that promote thrombosis and vascular dysfunction in cerebral endothelial cells.

In some aspects, the present disclosure addresses the diminishing levels of miR-19a, miR-21, and/or miR-146a microRNA after stroke by providing methods comprising administering a therapeutically effective combination of tPA along with mammalian exosomes and/or microvesicles; compositions containing the cargo of mammalian exosomes and/or microvesicles; or agents that induces the expression of at least one of miR-19a, miR-21, and/or miR-146a microRNA to increase levels of miR-19a, miR-21, or miR-146a microRNA in the brain and/or cerebral vasculature.

The targets of miRNA are recognized via a complementary site on the target mRNA. The miRNA binds to an Argonaute protein, and forms a silencing complex that targets a complementary mRNA through Watson-Crick pairing between the mRNA target region, and the miRNA (see Lewis et al., Conserved seed pairing, often flanked by adenosines, indicates that thousands of human genes are microRNA targets, Cell. 2005 Jan. 14; 120(1):15-20). The expression of some miR-19a, miR-21, or miR-146a microRNA targets is increased following stroke. Without wishing to be bound by any particular theory, it is believed that increasing levels of miR-19a, miR-21, or miR-146a microRNA in circulation and/or the brain enables the endothelial cells of blood vessels of the brain to decrease detrimental factors involved in stroke-induced cerebrovascular injury.

The present disclosure provides a method for treating stroke in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the treatment of stroke, wherein the combination of mammalian exosomes and tPA is for administration to a subject in need thereof in a therapeutically effective amount. The present disclosure provides a combination comprising mammalian exosomes and tPA for the manufacture of a medicament for the treatment of stroke, wherein the combination of mammalian exosomes and tPA is for administration to a subject in need thereof in a therapeutically effective amount.

The present disclosure provides a method for reducing the expansion of an ischemic core after stroke in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the reduction of the expansion of an ischemic core after stroke in a subject, wherein the combination of mammalian exosomes and tPA is for administration to a subject in need thereof in a therapeutically effective amount. The present disclosure provides a combination comprising mammalian exosomes and tPA for the manufacture of a medicament for the reduction of the expansion of an ischemic core after stroke in a subject, wherein the combination of mammalian exosomes and tPA is for administration to a subject in need thereof in a therapeutically effective amount.

The present disclosure provides a method for treating or preventing cerebrovascular injury in a subject, the method comprising administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of cerebrovascular injury, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of cerebrovascular injury, wherein the combination of mammalian exosomes and tPA is for administration to a subject in need thereof in a therapeutically effective amount.

The present disclosure provides a method for treating or preventing cerebrovascular injury in a subject, the method comprising administering a therapeutically effective amount of mammalian exosomes to and performing a thrombectomy on a subject in need thereof. The present disclosure provides mammalian exosomes for use in the treatment or prevention of cerebrovascular injury in a subject, wherein the treatment or prevention further comprises performing a thrombectomy. The present disclosure provides mammalian exosomes for the manufacture of a medicament for the treatment or prevention of stroke, wherein the mammalian exosomes are for administration to a subject in need thereof in a therapeutically effective amount, and wherein the treatment further comprises performing a thrombectomy.

The present disclosure provides a method for treating stroke in a subject, the method comprising administering a therapeutically effective amount of mammalian exosomes to and performing a thrombectomy on a subject in need thereof. The present disclosure provides mammalian exosomes for use in the treatment of stroke in a subject, wherein the treatment further comprises performing a thrombectomy, and wherein the mammalian exosomes are for administration to the subject in a therapeutically effective amount. The present disclosure provides mammalian exosomes for the manufacture of a medicament for the treatment of stroke, wherein the mammalian exosomes are for administration to a subject in need thereof in a therapeutically effective amount, and wherein the treatment further comprises performing a thrombectomy, and wherein the mammalian exosomes are for administration to the subject in a therapeutically effective amount.

The present disclosure provides a method for treating or preventing secondary thrombosis in downstream brain microvessels in a subject, the method comprising: administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of secondary thrombosis in downstream brain microvessels, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of secondary thrombosis in downstream brain microvessels, wherein the combination of mammalian exosomes and tPA is for administration to a subject in need thereof in a therapeutically effective amount.

The present disclosure provides a method for treating or preventing blood brain barrier impairment in a subject, the method comprising: administering a therapeutically effective amount of a combination of mammalian exosomes and Tissue Plasminogen Activator (tPA) to a subject in need thereof. The present disclosure provides a combination comprising mammalian exosomes and tPA for use in the treatment or prevention of blood brain barrier impairment, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of blood brain barrier impairment, wherein the combination of mammalian exosomes and tPA is for administration to a subject in need thereof in a therapeutically effective amount.

The present disclosure provides a method of treating or preventing cerebrovascular injury in a subject, the method comprising administering a therapeutically effective combination of mammalian exosomes and Tissue Plasminogen Activator (tPA) to and performing a thrombectomy on a subject in need thereof. The present disclosure provides a combination comprising mammalian exosomes and Tissue Plasminogen Activator (tPA) for use in the treatment or prevention of cerebrovascular injury in a subject, wherein the treatment or prevention further comprises performing a thrombectomy, and wherein the mammalian exosomes and tPA are for administration to the subject in a therapeutically effective amount. The present disclosure provides mammalian exosomes for use in the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides tissue Plasminogen Activator (tPA) for use in the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides a combination comprising mammalian exosomes and Tissue Plasminogen Activator (tPA) for use in the manufacture of a medicament for the treatment or prevention of cerebrovascular injury in a subject, wherein the treatment or prevention further comprises performing a thrombectomy, and wherein the mammalian exosomes and tPA are for administration to the subject in a therapeutically effective amount. The present disclosure provides mammalian exosomes for use in the manufacture of a medicament for the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy. The present disclosure provides tissue Plasminogen Activator (tPA) for use in the manufacture of a medicament for the treatment or prevention of cerebrovascular injury, the treatment or prevention comprising administering a combination comprising mammalian exosomes and Tissue Plasminogen Activator (tPA), and wherein the treatment or prevention further comprises performing a thrombectomy.

The present disclosure provides a method of treating stroke in a subject, the method comprising administering a therapeutically effective combination of mammalian exosomes and tPA to and performing a thrombectomy on a subject in need thereof. The present disclosure provides a combination comprising mammalian exosomes and tPA for use in the treatment of stroke in a subject, wherein the treatment further comprises performing a thrombectomy, and wherein the mammalian exosomes and tPA are for administration to the subject in a therapeutically effective amount. The present disclosure provides mammalian exosomes for use in the treatment of stroke, the treatment comprising administering a combination comprising mammalian exosomes and tPA, and wherein the treatment further comprises performing a thrombectomy. The present disclosure provides tPA for use in the treatment of stroke, the treatment comprising administering a combination comprising mammalian exosomes and tPA, and wherein the treatment further comprises performing a thrombectomy. The present disclosure provides a combination comprising mammalian exosomes and tPA for use in the manufacture of a medicament for the treatment of stroke in a subject, wherein the treatment further comprises performing a thrombectomy, and wherein the mammalian exosomes and tPA are for administration to the subject in a therapeutically effective amount. The present disclosure provides mammalian exosomes for use in the manufacture of a medicament for the treatment of, the treatment comprising administering a combination comprising mammalian exosomes and tPA, and wherein the treatment further comprises performing a thrombectomy. The present disclosure provides tPA for use in the manufacture of a medicament for the treatment of stroke, the treatment comprising administering a combination comprising mammalian exosomes and tPA, and wherein the treatment further comprises performing a thrombectomy.

The present disclosure provides a method for treating or preventing of blood brain barrier leakage in a subject, the method comprising administering a therapeutically effective amount of a combination comprising mammalian exosomes and Tissue Plasminogen Activator (tPA) to a subject in need thereof. The present disclosure provides a combination comprising mammalian exosomes and tPA for use in the treatment or prevention of blood brain barrier leakage, wherein the combination of mammalian exosomes and tPA is for administration to a subject in need thereof in a therapeutically effective amount. The present disclosure provides a combination comprising mammalian exosomes and tPA for the manufacture of a medicament for the treatment or prevention of blood brain barrier leakage, wherein the combination of mammalian exosomes and tPA is for administration to a subject in need thereof in a therapeutically effective amount.

The present disclosure provides a method for treating stroke in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the treatment of stroke, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment of stroke, wherein the combination of mammalian exosomes and at least one thrombolytic agent is for administration to a subject in need thereof in a therapeutically effective amount.

The present disclosure provides a method for reducing the expansion of an ischemic core after stroke in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the reduction of the expansion of an ischemic core after stroke in a subject, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the reduction of the expansion of an ischemic core after stroke in a subject, wherein the combination of mammalian exosomes and at least one thrombolytic agent is for administration to a subject in need thereof in a therapeutically effective amount.

The present disclosure provides a method for treating or preventing cerebrovascular injury in a subject, the method comprising administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of cerebrovascular injury, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of cerebrovascular injury, wherein the combination of mammalian exosomes and at least one thrombolytic agent is for administration to a subject in need thereof in a therapeutically effective amount.

The present disclosure provides a method for treating or preventing cerebrovascular injury in a subject, the method comprising administering a therapeutically effective amount of mammalian exosomes to and performing a thrombectomy on a subject in need thereof. The present disclosure provides mammalian exosomes for use in the treatment or prevention of cerebrovascular injury in a subject, wherein the treatment or prevention further comprises performing a thrombectomy. The present disclosure provides mammalian exosomes for the manufacture of a medicament for the treatment or prevention of stroke, wherein the mammalian exosomes are for administration to a subject in need thereof in a therapeutically effective amount, and wherein the treatment further comprises performing a thrombectomy.

The present disclosure provides a method for treating stroke in a subject, the method comprising administering a therapeutically effective amount of mammalian exosomes to and performing a thrombectomy on a subject in need thereof. The present disclosure provides mammalian exosomes for use in the treatment of stroke in a subject, wherein the treatment further comprises performing a thrombectomy, and wherein the mammalian exosomes are for administration to the subject in a therapeutically effective amount. The present disclosure provides mammalian exosomes for the manufacture of a medicament for the treatment of stroke, wherein the mammalian exosomes are for administration to a subject in need thereof in a therapeutically effective amount, and wherein the treatment further comprises performing a thrombectomy, and wherein the mammalian exosomes are for administration to the subject in a therapeutically effective amount.

The present disclosure provides a method for treating or preventing secondary thrombosis in downstream brain microvessels in a subject, the method comprising: administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of secondary thrombosis in downstream brain microvessels, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of secondary thrombosis in downstream brain microvessels, wherein the combination of mammalian exosomes and at least one thrombolytic agent is for administration to a subject in need thereof in a therapeutically effective amount.

The present disclosure provides a method for treating or preventing blood brain barrier impairment in a subject, the method comprising: administering a therapeutically effective amount of a combination of mammalian exosomes and at least one thrombolytic agent to a subject in need thereof. The present disclosure provides a combination comprising mammalian exosomes and at least one thrombolytic agent for use in the treatment or prevention of blood brain barrier impairment, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of blood brain barrier impairment, wherein the combination of mammalian exosomes and at least one thrombolytic agent is for administration to a subject in need thereof in a therapeutically effective amount.

The present disclosure provides a method of treating or preventing cerebrovascular injury in a subject, the method comprising administering a therapeutically effective combination of mammalian exosomes and at least one thrombolytic agent to and performing a thrombectomy on a subject in need thereof. The present disclosure provides a combination comprising mammalian exosomes and at least one thrombolytic agent for use in the treatment or prevention of cerebrovascular injury in a subject, wherein the treatment or prevention further comprises performing a thrombectomy, and wherein the mammalian exosomes and at least one thrombolytic agent are for administration to the subject in a therapeutically effective amount. The present disclosure provides mammalian exosomes for use in the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides at least one thrombolytic agent for use in the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides a combination comprising mammalian exosomes and at least one thrombolytic agent for use in the manufacture of a medicament for the treatment or prevention of cerebrovascular injury in a subject, wherein the treatment or prevention further comprises performing a thrombectomy, and wherein the mammalian exosomes and at least one thrombolytic agent are for administration to the subject in a therapeutically effective amount. The present disclosure provides mammalian exosomes for use in the manufacture of a medicament for the treatment or prevention of cerebrovascular injury, the treatment or prevention comprising administering a combination comprising mammalian exosomes and at least one thrombolytic agent, and wherein the treatment or prevention further comprises performing a thrombectomy. The present disclosure provides at least one thrombolytic agent for use in the manufacture of a medicament for the treatment or prevention of cerebrovascular injury, the treatment or prevention comprising administering a combination comprising mammalian exosomes and at least one thrombolytic agent, and wherein the treatment or prevention further comprises performing a thrombectomy.

The present disclosure provides a method of treating stroke in a subject, the method comprising administering a therapeutically effective combination of mammalian exosomes and at least one thrombolytic agent to and performing a thrombectomy on a subject in need thereof. The present disclosure provides a combination comprising mammalian exosomes and at least one thrombolytic agent for use in the treatment of stroke in a subject, wherein the treatment further comprises performing a thrombectomy, and wherein the mammalian exosomes and at least one thrombolytic agent are for administration to the subject in a therapeutically effective amount. The present disclosure provides mammalian exosomes for use in the treatment of stroke, the treatment comprising administering a combination comprising mammalian exosomes and at least one thrombolytic agent, and wherein the treatment further comprises performing a thrombectomy. The present disclosure provides at least one thrombolytic agent for use in the treatment of stroke, the treatment comprising administering a combination comprising mammalian exosomes and at least one thrombolytic agent, and wherein the treatment further comprises performing a thrombectomy. The present disclosure provides a combination comprising mammalian exosomes and at least one thrombolytic agent for use in the manufacture of a medicament for the treatment of stroke in a subject, wherein the treatment further comprises performing a thrombectomy, and wherein the mammalian exosomes and at least one thrombolytic agent are for administration to the subject in a therapeutically effective amount. The present disclosure provides mammalian exosomes for use in the manufacture of a medicament for the treatment of, the treatment comprising administering a combination comprising mammalian exosomes and at least one thrombolytic agent, and wherein the treatment further comprises performing a thrombectomy. The present disclosure provides at least one thrombolytic agent for use in the manufacture of a medicament for the treatment of stroke, the treatment comprising administering a combination comprising mammalian exosomes and at least one thrombolytic agent, and wherein the treatment further comprises performing a thrombectomy.

The present disclosure provides a method for treating or preventing of blood brain barrier leakage in a subject, the method comprising administering a therapeutically effective amount of a combination comprising mammalian exosomes and at least one thrombolytic agent to a subject in need thereof. The present disclosure provides a combination comprising mammalian exosomes and at least one thrombolytic agent for use in the treatment or prevention of blood brain barrier leakage, wherein the combination of mammalian exosomes and at least one thrombolytic agent is for administration to a subject in need thereof in a therapeutically effective amount. The present disclosure provides a combination comprising mammalian exosomes and at least one thrombolytic agent for the manufacture of a medicament for the treatment or prevention of blood brain barrier leakage, wherein the combination of mammalian exosomes and at least one thrombolytic agent is for administration to a subject in need thereof in a therapeutically effective amount.

Any of the preceding methods can further comprise performing a thrombectomy on the subject.

In some aspects of the preceding methods, treating, preventing or treating or preventing can comprise any one of the following or any combination of the following: (a) increasing proteolysis of fibrin in a clot and/or thrombus, (b) increasing the rate and extent of vessel recanalization, (c) increasing microvascular reperfusion without increasing brain hemorrhage, (d) reducing leakage of the blood-brain-barrier, (e) attenuating infarct expansion, (f) reducing prothrombotic procoagulant vascular conditions, (g) reducing vascular and/or cerebral brain cell inflammation, (h) reducing prothrombotic procoagulant vascular conditions and vascular and subsequent cerebral brain cell inflammation, (i) extending the therapeutic window for tPA treatment, (j), reducing the size of a clot or thrombus, (k) reducing adhesion molecules, (l) reducing vascular inflammation, (m) reducing procoagulant and/or prothrombotic conditions, (n) reducing the expansion of an ischemic core, (o) reducing infarct volume, (p) improving neurological outcome, (q) enhancing tissue perfusion, (r) extending the therapeutic window for treatment with at least one thrombolytic agent.

The present disclosure provides methods for treating stroke and methods for the treatment and prevention of cerebrovascular injury associated with stroke, comprising administering a therapeutically effective amount of a combination comprising mammalian exosomes and/or microvesicles and Tissue Plasminogen Activator (tPA) to a subject in need thereof. The present disclosure also provides methods for treating stroke and methods for the treatment and prevention of cerebrovascular injury associated with stroke, the methods comprising administering a therapeutically effective amount of mammalian exosomes and/or microvesicles and performing thrombectomy in a stroke patient in need thereof.

The present disclosure provides methods for treating and/or preventing secondary stroke and methods for the treatment and prevention of cerebrovascular injury associated with secondary stroke, comprising administering a therapeutically effective amount of a combination comprising mammalian exosomes and/or microvesicles and Tissue Plasminogen Activator (tPA) to a subject in need thereof. The present disclosure also provides methods for treating and/or preventing secondary stroke and methods for the treatment and prevention of cerebrovascular injury associated with secondary stroke, the methods comprising administering a therapeutically effective amount of mammalian exosomes and/or microvesicles and performing thrombectomy in a stroke patient in need thereof.

In some embodiments, the mammalian exosomes and/or microvesicles contain at least one or more of the following microRNA: miR-19a, miR-21, and miR-146a microRNA, or agents that induce the expression of miR-19a, miR-21, and/or miR-146a microRNA in the endothelial cells of the brain vasculature of the subject in need thereof.

Thus, methods of the present disclosure shall be taken to apply mutatis mutandis to methods for preventing a cerebrovascular injury in a subject who has suffered a neurological ischemic event, for example, an ischemic stroke.

Ischemic stroke can be diagnosed based on the presentation of certain symptoms, in concert with the subject's history, a physical examination, serum glucose levels, oxygen saturation levels, and a noncontrast CT scan. Symptoms of stroke include general symptoms such as sudden weakness, paralysis, numbness, confusion, trouble speaking or understanding speech, trouble seeing in one or both eyes, problems breathing, dizziness, trouble walking, loss of balance or coordination, and unexplained falls, loss of consciousness, and/or sudden and severe headache. More specifically, stroke symptoms include the following: carotid distribution; hemiparesis or monoparesis; hemisensory numbness or neglect; facial weakness; aphasia; dysarthria; vertigo; amaurosis fugax (fleeting blindness of one eye); vertebrobasilar distribution; dysarthria; dysphagia; diplopia; homonymous hemianopsia; total blindness (cortical blindness); alternating or bilateral weakness; alternating or bilateral numbness; “crossed” weakness or numbness (ipsilateral face and contralateral body); gait ataxia; and/or limb dysmetria.

“Identifying” or “selecting a subject having a stroke” or a “subject in need thereof” means identifying or selecting a subject with a stroke; or, identifying or selecting a subject having any one or more symptoms of a stroke or symptoms of a cerebrovascular injury, including, but not limited to, general symptoms such as sudden weakness, paralysis, numbness, confusion, trouble speaking or understanding speech, trouble seeing in one or both eyes, problems breathing, dizziness, trouble walking, loss of balance or coordination, and unexplained falls, loss of consciousness, and/or sudden and severe headache. More specifically, stroke symptoms include the following: carotid distribution; hemiparesis or monoparesis; hemisensory numbness or neglect; facial weakness; aphasia; dysarthria; vertigo; amaurosis fugax (fleeting blindness of one eye); vertebrobasilar distribution; dysarthria; dysphagia; diplopia; homonymous hemianopsia; total blindness (cortical blindness); alternating or bilateral weakness; alternating or bilateral numbness; “crossed” weakness or numbness (ipsilateral face and contralateral body); gait ataxia; limb dysmetria; and/or any combination of these, or other symptom enumerated above. Such identification may be accomplished by any method, including but not limited to, standard clinical tests or assessments, such as measuring serum or circulating (plasma) cholesterol, measuring serum or circulating (plasma) blood-glucose, measuring serum or circulating (plasma) triglycerides, measuring blood-pressure, measuring body fat content, measuring body weight, physical examination, oxygen saturation levels, noncontrast CT scan, angiogram, and the like

The hallmark feature of ischemic stroke is a sudden loss of focal brain function; however, before administering the combination of mammalian exosomes and/or microvesicles and tPA and/or thrombectomy as described herein, conditions other than brain ischemia presenting similar symptoms should be ruled out. Accordingly, the differential diagnoses for ischemic stroke that should be evaluated and ruled out prior to administration include migraine aura; cerebral venous thrombosis; functional deficit (conversion reaction); seizure with postictal paresis (Todd paralysis), aphasia, or neglect; central nervous system tumor or abscess; head trauma; hypertensive encephalopathy; mitochondrial disorder (e.g., mitochondrial encephalopathy with lactic acidosis and stroke-like episodes or MELAS); posterior reversible encephalopathy syndrome (PRES); multiple sclerosis; reversible cerebral vasoconstriction syndromes (RCVS); spinal cord disorder (e.g., compressive myelopathy, spinal dural arteriovenous fistula); subdural hematoma; syncope; systemic infection; toxic-metabolic disturbance (e.g., hypoglycemia, exogenous drug intoxication); transient global amnesia; viral encephalitis (e.g., herpes simplex encephalitis); and/or Wernicke encephalopathy (see Kothari R, et al. Early stroke recognition: developing an out-of-hospital NIH Stroke Scale. Acad. Emerg. Med. 1997; 4:986).

The first-line therapy for ischemic stroke is intravenous thrombolysis and/or endovascular thrombectomy; however, thrombolytic therapy has hitherto been approached cautiously due to the risk of intracerebral hemorrhage (see NINDS, Tissue plasminogen activator for ischemic stroke. 2015; N. Engl. J. Med. 333 1581-1587). A thrombus (also known as a blood clot) forms in response to a thrombin-mediated fibrin formation and platelet activation (Del Zoppo G J, Thrombolytic therapy in cerebrovascular disease, Stroke. 1988 September; 19(9):1174-9).

In some embodiments, a subject who has suffered an ischemic stroke is treated with a therapeutically effective combination comprising mammalian exosomes and/or microvesicles and tPA, and/or thrombectomy, to prevent or treat the symptoms of stroke and/or cerebrovascular injury which occurs as a result of the stroke, or the underlying causes of both. In various embodiments, mammalian exosomes and/or microvesicles are administered in therapeutically effective amounts in combination with tPA and/or thrombectomy to prevent and/or treat a cerebrovascular injury, wherein the cerebrovascular injury is selected from: congested and/or engorged grey matter; disrupted blood-brain-barrier; dysregulation of blood pressure; disruption of cerebral blood flow; reduced cerebral blood perfusion; inhibition of neuronal protein synthesis; disruption of neuronal glucose utilization; tissue acidosis; neuronal electrical failure; brain tissue necrosis; neuronal cell apoptosis; neuronal cell necrosis; depletion of adenosine triphosphate (ATP); changes in ionic concentrations of sodium, potassium, and calcium in the brain; increased lactate in the brain; acidosis; accumulation of oxygen free radicals in the brain; intracellular accumulation of water in the brain; activation of proteolytic processes in neuronal and neuronal-support cells; increase release of glutamate at neuronal synapses, and the downstream activation of glutamate receptors, and subsequent ionic disruption; increased production of reactive oxygen species; inflammation; loss of structural integrity in the brain; and/or cerebral edema (cytotoxic or vasogenic).

Thrombolysis is a treatment wherein protein members of the fibrinolytic system remove the offending thrombus; this is achieved when plasminogen activators (e.g., tPA) catalyze the proenzyme plasminogen into plasmin, the enzyme that subsequently degrades the fibrin that constitutes the thrombus (Collen D and Lijnen H R, New approaches to thrombolytic therapy, Arteriosclerosis, 1984 November-December; 4(6):579-85). Plasminogen activators such as tPA have been purified, and are used to lyse the thrombus in cases of ischemic stroke.

Administration of mammalian exosomes and tPA can occur concomitantly, or sequentially, for example, with mammalian exosomes being administered upon the presentation of stroke symptoms and tPA being administered after; with tPA being administered followed by the administration of mammalian exosomes; or tPA and mammalian exosomes may be administered at the same time. Thus, in some embodiments, a therapeutically effective dose of mammalian exosomes will be administered before, after, and/or at the same time as tPA; the tPA being administered at a therapeutically effective dose, or at a suboptimal dose. In some embodiments, a therapeutically effective dose of mammalian exosomes can be administered immediately upon the presentation of any one or more of the stroke symptoms enumerated above, and/or at any time point afterwards up until the conclusion of treatment. In some embodiments, a standard time window for tPA administration may include (when administered subsequent to the administration of the exosomes of the present disclosure), from about 0.1 hours after onset of stroke symptoms to about 12 hours after onset of stroke symptoms, for example, from about 0.1 hours to about 10 hours, or from about 0.1 hours to about 8 hours, or from about 0.1 hours to about 6 hours, or for example extends the therapeutic window of tPA by 6 to 9 hours from the onset of stroke symptoms. In the example above, tPA may be dosed concurrently with the exosomes or subsequent to the administration of the exosomes. In various embodiments, the present regimen to treat a stroke patient may include dosing the exosomes from about 0.1 hrs to about 3 hours after the onset of stroke symptoms, and then dosing of the tPA from about 0 hrs to about 12 hours after the dosing of the exosomes. It is to be understood, that the administration of the exosomes prior to the administration of the tPA provides significant advantages over the standard of care, wherein the therapeutic window for the administration of tPA for example Alteplase (Rx), to a stroke patient without dosing the exosomes is about 3 hours, but may be a long as 3-4.5 hours in humans. In various embodiments, a patient having experienced an ischemic stroke may be dosed with the exosomes of the present invention concurrently with the tPA, or prior to the administration of tPA and extend the therapeutic window for dosing tPA to about 0.5 hrs to about 12 hours or more when compared to the average therapeutic window of tPA of 3-4.5 hours. When the tPA is dosed after the exosomes, the tPA may be dosed using a therapeutically effective amount as soon as stroke symptoms are detected, i.e. within 10-30 minutes, and the therapeutic window is extended to about 6-9 hours or for example, at least 8 hours after the onset of stroke. For example, in a subject that has suffered a stroke, and/or is presenting one or more of the stroke symptoms enumerated above, the therapeutically effective dose of mammalian exosomes may be administered immediately upon presentation of the stroke symptoms (i.e., observations from the subject and/or witnesses to the possible ischemic event); upon a physical examination findings such as absent pulses (inferior extremity, radial, or carotid), the presence of a neck bruit, loss of facial pulse on the presumptive side of occlusion, and/or increased facial pulse on the side of occlusion, atrial fibrillation, murmurs and cardiac enlargement, cholesterol crystal, white platelet-fibrin, or red clot emboli on the optic fundus, subhyaloid hemorrhages in the eye, speckled iris, a dilated and poorly reactive ipsilateral pupil, and/or venous stasis retinopathy; upon the presentation of neurological findings such as weakness of the face, arm, and leg on one side of the body unaccompanied by sensory, visual, or cognitive abnormalities, large focal neurologic deficits that begin abruptly or progress quickly, abnormalities of language, the presence of motor and sensory signs on the same side of the body, vertigo, staggering, diplopia, deafness, crossed symptoms (i.e., one side of the face and other side of the body), bilateral motor and/or sensory signs, and/or hemianopsia, and/or the sudden onset of impaired consciousness in the absence of focal neurologic signs; upon the presentation of certain biomarkers such as serum D-dimer levels; immediately after the presentation of one or more of the aforementioned stroke symptoms and/or clinical presentations; concurrently with the administration of tPA (whether the tPA administration occurs upon presentation of stroke symptoms or at the end of the available tPA treatment window); subsequent to the administration of tPA; at the end of tPA treatment; and/or as a follow-up, subsequent dose, or completing dose for a given course of treatment.

As used herein, the term “course of treatment” refers to a period of continual treatment, sometimes with variable dosage and/or in combination with other modalities, and/or at varying time points. For example, in some embodiments, a course of treatment in regard to mammalian exosomes may refer to an initial dose of mammalian exosomes upon the presentation of one or more stroke symptoms, with a subsequent follow-up dose occurring some period of time later. Alternatively, in some embodiments, the course of treatment may include an initial dose of mammalian exosomes, followed by sequentially tapered doses of mammalian exosomes. However, additional doses of exosomes can be administered 24 h after the prior dose based on the fact that half-life of exosomes is approximately 24 h when administered to a human. In some embodiments, a therapeutically effective dose of mammalian exosomes may be administered prophylactically to an individual with a high risk of stroke. For example, prophylactic mammalian exosomes may be administered to an at risk individual with conditions such as arteriovenous malformation; cavernous angioma; cerebral amyloid angiopathy; atherosclerosis, especially in the elderly; hypertension; obesity; dyslipidemia; glucose intolerance; metabolic syndrome; heart disease, including cardiac valvular disease, prior myocardial infarction, atrial fibrillation, and endocarditis; and/or individuals who smoke, use amphetamines, or cocaine; in anticipation of activities and/or procedures likely to increase the risk of stroke such as surgery; activities likely to cause carotid/vertebral dissection injuries such as operating high speed vehicles, high-impact sports such as football or hockey, or combat. Administering exosomes prophylactically to persons at risk, for example, elderly patients having a condition that makes them more likely to experience a stroke e.g. atrial fibrillation, can be very valuable.

As used herein, the term “risk” or “at-risk individual” refers to the possibility or the chance of an individual developing a particular disease, disorder, or condition based on one or more risk factors, which are measurable parameters that correlate with development of a particular disease, disorder, or condition, as known in the art, at a higher probability than an individual without one or more of these risk factors. Here, an at-risk individual may or may not have detectable disease or symptoms of disease, and may or may not have displayed detectable disease or symptoms of disease prior to the treatment methods described herein.

The administration of mammalian exosomes and tPA creates a therapeutically effective combination that acts to enhance the fibrinolytic effect of tPA, extend the therapeutic window for thrombolysis, promote vessel recanalization, augment microvascular reperfusion without increased brain hemorrhage, prevent blood-brain-barrier leakage, and attenuate infarct expansion in a subject who has suffered a stroke.

In some embodiments, the thrombolytic agent Alteplase will be administered before, after, and/or at the same time as a therapeutically effective dose of mammalian exosomes to a patient who is presenting symptoms of ischemic stroke. When treating with Alteplase, the recommended total dose of Alteplase is 0.9 mg/kg per subject's body weight, with a maximum total dose of 90 mg. Subject's weighing less than or equal to 100 kg can be treated by loading 0.09 mg/kg (10% of 0.9 mg/kg dose) as an IV bolus for 1 minute, followed by 0.81 mg/kg (90% of 0.9 mg/kg dose) supplied as a continuous infusion over an hour. Subjects weighing more than 100 kg can be treated by loading 9 mg (10% of 90 mg) as an IV bolus for 1 minute, followed by 81 mg (90% of 90 mg) supplied as a continuous infusion over an hour.

The effect of a suboptimal dose of Alteplase (i.e., 0.6 mg/kg) has been shown to produce comparable results to standard-dose Alteplase (i.e., 0.9 mg/kg) (see Robinson T G, et al., Low Versus Standard-Dose Alteplase in Patients on Prior Antiplatelet Therapy: The ENCHANTED Trial (Enhanced Control of Hypertension and Thrombolysis Stroke Study). Stroke. 2017; 48(7):1877-83). In some embodiments, a suboptimal dose of Alteplase will be administered before, after, and/or at the same time as a therapeutically effective dose of mammalian exosomes.

In some embodiments, a therapeutically effective dose of mammalian exosomes can be administered prior to, during, or after intra-arterial thrombolytic therapy, wherein tPA is infused locally and/or in close proximity to the thrombus. For example, a soft, small diameter microcatheter can be navigated to the thrombus, and a lower dose (suboptimal dose) of thrombolytic agents, and/or mammalian exosomes can be directly delivered to the thrombus.

In some embodiments, a therapeutically effective dose of mammalian exosomes can be administered prior to, during, or after a combination of intra-arterial thrombolytic therapy (e.g., tPA) and intravenous thrombolytic therapy (IA/IV), wherein tPA is infused locally and/or in close proximity to the thrombus, and administered via IV. For example, a soft, small diameter microcatheter can be navigated to the thrombus, and a lower dose (suboptimal dose) of thrombolytic agents, and/or mammalian exosomes can be directly delivered to the thrombus, while the individual is simultaneously administered IV tPA.

As used herein, the term “thrombectomy” refers to any procedure wherein a thrombus is removed. For example, the term thrombectomy can describe the removal of a thrombus by several different means, including but not limited to the use of techniques and tools pertaining to percutaneous thrombectomy or mechanical thrombectomy, such as simple catheters and guidewires, stent retrievers, coil retrievers, aspiration devices, balloon maceration devices, hydrodynamic devices, acoustic energy devices, spinning brush devices, spinning wire devices, or open surgery.

Thrombectomy offers an alternative to thrombolysis, and, in some situations, may have advantages. For example, thrombectomy may be superior to tPA in cases of large artery occlusion; where the thrombus is resistant to thrombolysis; and/or may be available as a treatment after the available window for tPA treatment has passed. Furthermore, a thrombectomy may be performed in concert, or as an adjuvant to tPA.

In some embodiments, mammalian exosomes may be administered before, after, or concomitantly with thrombectomy, after the unsuccessful treatment with tPA (i.e., failure for tPA to induce recanalization); in other embodiments.

In some embodiments, mammalian exosomes may be administered before, after, or concomitantly with a retrievable stent thrombectomy procedure, such as the Solitaire™ FR Revascularization Device as follows: first, a neurological examination is given prior to the procedure. Next, an ENVOY 6F guide catheter (DePuy Synthes) is inserted into the right internal carotid artery, and is navigated using standard imaging techniques. Contrast is then injected to identify the location of occlusion. A 2.3F Codman Neurovascular PROWLER SELECT PLUS microcatheter is then inserted into the guide catheter, which can be advanced with a 0.014 Stryker Neurovascular Syncrho2 guide wire into the artery. The microcatheter is then advanced across the thrombus, and the microwire is removed. Next, a 6×20 mm Solitaire FR revascularization device is advanced into the PROWLER SELECT PLUS microcatheter, followed by advancing the stent retriever into the microcatheter. After it is advanced, the Solitaire FR revascularization device is deployed across the thrombus, followed by deployment of the stent retriever and thrombus integration. A 60 cc syringe is attached to a side port to provide continuous suction, and the Solitaire FR revascularization device is retracted with the microcatheter under negative pressure via continuous suction, thus retrieving the thrombus. Contrast is then once again injected to determine recanalization, and the ENVOY 6F guide catheter is removed. Finally, a neurological examination is performed to assess the outcome of the procedure.

In some embodiments, mammalian exosomes may be administered before, after, or concomitantly with a thrombectomy procedure using the Merci Retrieval System (Concentric Medical), which consists of a retriever (5 helical loops of decreasing diameter, from 2.8 mm to 1.1 mm), balloon guide catheter (9F catheter with 2.1-mm lumen and a balloon located at the distal tip) and microcatheter. For example, prior to, during, or after the thrombectomy procedure, a therapeutically effective dose of mammalian exosomes will be administered. The thrombectomy procedure in this example is as follows: first, the subject will be given a bolus of 3000 U of intravenous heparin. Next, the balloon guide catheter is placed into the subclavian artery (i.e., for posterior circulation occlusion), or the common or internal carotid artery (i.e., for anterior circulation occlusion). The microcatheter is then guided into the occluded vessel, and advanced beyond the thrombus using standard cerebral catheterization techniques known to those in the art. Prior to the deployment of the Merci Retriever, a selective angiogram should be performed distal to the thrombus in order to determine and evaluate the tortuosity and size of the distal arteries. The Merci Retriever is then advanced through the microcatheter, with two to three helical loops deployed past the thrombus. Upon contact with the thrombus, the proximal loops of the Merci Retriever are deployed. Five clockwise rotations are made on the Merci Retriever to ensnare the thrombus, and, during removal of the thrombus, the balloon guide catheter inflated to control intracranial blood flow. Once the thrombus is ensnared, the Merci Retriever and microcatheter are withdrawn as one unit into the lumen of the balloon guide catheter, with continuous aspiration applied to ensure the thrombus is completely removed. Blood flow is re-established by deflating the balloon, and removal of the thrombus is confirmed by brisk reflux of blood and repeat angiogram findings (see Gobin et al., MERCI 1: A Phase 1 Study of Mechanical Embolus Removal in Cerebral Ischemia, Stroke. 2004; 35: 2848-2854).

In some embodiments, mammalian exosomes may be administered before, after, or concomitantly with a thrombectomy procedure, such as the Penumbra thrombectomy system. For example, mammalian exosomes exemplified herein, may be administered before, during, and/or after a procedure as follows: access the artery is achieved by performing percutaneous techniques under fluoroscopic guidance known to those in the art. The vascular occlusion of the individual's angioarchitecture is determined using four-vessel digital subtraction angiography. Next, a guide catheter selected based on the individual's morphology is selected and guided into the target vessel, thus allowing access to the Penumbra reperfusion catheter. After the catheter has been advanced to a location proximal to the clot, the guidewire is removed from the Penumbra reperfusion catheter, and the Penumbra separator is advanced therein. To initiate revascularization, the Penumbra aspiration pump is activated, and the thrombus is reduced by connecting the reperfusion catheter to the Penumbra aspiration pump (generating a vacuum of −20 inches/Hg). Continuously advancing and withdrawing the separator through the reperfusion catheter into the thrombus facilitates an aspiration/debulking process. Any remaining thrombus is removed via a secondary method of direct mechanical retrieval using a thrombus removal ring; here, the thrombus is extracted by engaging the proximal portion of the thrombus, and extracting it under flow arrest conditions (i.e., by inflating a proximal balloon guide catheter) (see Penumbra Pivotal Stroke Trial I. The penumbra pivotal stroke trial: safety and effectiveness of a new generation of mechanical devices for clot removal in intracranial large vessel occlusive disease. Stroke. 2009; 40(8):2761-8).

The amount of mammalian exosomes and tPA, and/or the thrombectomy procedure performed, in the exemplified compositions, and formulations, whether pharmaceutically acceptable or not, may vary according to factors such as the type of disease, state, age, sex, and weight of the individual. Dosage regimens may be adjusted to provide the optimum therapeutic response. For example, a single bolus of mammalian exosomes (e.g., a single bolus of exosomes, or compositions containing the contents of said exosomes, or a single bolus of microvesicles, or compositions containing the contents of said microvesicles) may be administered, several divided doses may be administered over time, or the dose may be proportionally reduced or increased as indicated by the exigencies of the therapeutic situation. It is especially advantageous to formulate parenteral compositions (for example by intravenous, intraarterially, intraperitoneal, intranasal, subcutaneous, or other known routes for delivery of cells or components thereof) in a dosage unit form for ease of administration and uniformity of dosage. Dosage unit form as used herein refers to physically discrete units suited as unitary dosages for the mammalian subjects to be treated; each unit containing a predetermined quantity of active compound (e.g., mammalian exosomes or microvesicles with or without a miR-19a, miR-21, and/or miR-146a microRNA) calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The composition of the invention can be delivered to the subject at a dose that is effective to treat and/or prevent cerebrovascular injury, disease or disorders, and/or the symptoms of stroke. The effective dosage will depend on many factors including the gender, age, weight, and general physical condition of the subject, the severity of the symptoms, the particular compound or composition being administered, the duration of the treatment, the nature of any concurrent treatment, the carrier used, and like factors within the knowledge and expertise of those skilled in the art. As appropriate, a treatment effective amount in any individual case can be determined by one of ordinary skill in the art by reference to the pertinent texts and literature and/or by using routine experimentation (see, e.g., Remington, The Science and Practice of Pharmacy (21st ed. 2005)).

In one embodiment, mammalian exosomes and/or microvesicles, (for example, exosomes and/or microvesicles containing one or more of miR-19a, miR-21, and miR-146a microRNA) is administered to a subject in need thereof (i.e., who has had a stroke), at a dose of about 0.0001 μg/kg to about 900 μg/kg; 0.005 μg/kg to about 500 μg/kg; 0.01 μg/kg to about 100 μg/kg; 0.1 μg/kg to about 50 μg/kg; and encompasses every sub-range within the cited ranges and amounts.

In another embodiment, mammalian exosomes and/or microvesicles are administered at a dose of about 1×10⁵ to about 1×10¹⁷ mammalian cell derived exosomes and/or microvesicles per kg of body weight of the subject, or 1×10⁵ to about 1×10¹⁶ mammalian cell derived exosomes and/or microvesicles per kg of body weight of the subject, or 1×10⁶ to about 1×10¹⁵ mammalian cell derived exosomes and/or microvesicles per kg of body weight of the subject, or 1×10⁷ to about 1×10¹⁴ mammalian cell derived exosomes and/or microvesicles per kg of body weight of the subject, or 1×10⁸ to about 1×10¹³ mammalian cell derived exosomes and/or microvesicles per kg of body weight of the subject, or 1×10⁹ to about 1×10¹² mammalian cell derived exosomes and/or microvesicles per kg of body weight of the subject, or 1×10¹ to about 1×10¹⁷ mammalian cell derived exosomes and/or microvesicles per kg of body weight of the subject, or 1×10¹ to about 1×10¹⁶ mammalian cell derived exosomes and/or microvesicles per kg of body weight of the subject, or 1×10¹ to about 1×10¹⁵ mammalian cell derived exosomes and/or microvesicles per kg of body weight of the subject, or 1×10¹ to about 1×10¹⁴ mammalian cell derived exosomes and/or microvesicles per kg of body weight of the subject, or 1×10¹ to about 1×10¹³ mammalian cell derived exosomes and/or microvesicles per kg of body weight of the subject, or 1×10¹ to about 1×10¹² mammalian cell derived exosomes and/or microvesicle per kg of body weight of the subject.

In some embodiments, the mammalian exosomes are administered at a dose of about 1×10¹⁰ to about 1×10¹⁹, or about 1×10¹¹ to about 1×10¹⁸, or about 1×10¹² to about 1×10¹⁷, or about 1×10¹³ to about 1×10¹⁶, mammalian cell derived exosomes and/or microvesicles per dose, once or multiple times per day, or once or multiple times per week, or once or multiple times per month. In various embodiments, the exemplified doses of mammalian cell derived exosomes and/or microvesicles per kg weight of the patient are daily doses or therapeutically effective doses, either in unit form or in sub-unit forms to be dosed one or more times per day, or one or more times per week, or one or more times per month.

In each of the above referenced mammalian exosomes and/or microvesicle dosages, the mammalian cells that can be used to isolate the exosomes and/or microvesicles can include: cells that are known to produce exosomes, and microvesicles, for example, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (VIDDCs), monocytes, B lymphocytes, antigen-presenting cells, glial cells, astrocytes, neurons, oligodendrocytes, spindle neurons, microglia, or mastocytes.

In one embodiment of the invention, the subject is one that has suffered a stroke, and has developed a cerebrovascular injury, and is administered a therapeutic combination (i.e., mammalian exosomes and/or microvesicles, tPA, and/or thrombectomy) of the present disclosure after the development of the cerebrovascular injury in order to ameliorate and/or relieve the symptoms, or the severity of the symptoms of the stroke and/or cerebrovascular injury. In another embodiment, the subject is one that has suffered a stroke, and has not developed a cerebrovascular injury, and the therapeutic combination (i.e., mammalian exosomes and/or microvesicles, tPA, and/or thrombectomy) is administered to the subject to prevent the development of cerebrovascular injury or symptoms thereof. Accordingly, the composition of the invention can be delivered to the subject prior to the event occurring (i.e., a cerebrovascular injury and/or a stroke); concurrently with the event; and/or after the event occurs but before the development of cerebrovascular injury symptoms, or after the event occurs and after the development of cerebrovascular injury symptoms.

Thrombolytic agents such as tPA lyse and/or dissolve blood clots by activating plasminogen, which when cleaved forms proteolytic enzyme called plasmin. Plasmin exerts its effect by breaking fibrin cross-links, thus disrupting the structural integrity of blood clots. In some non-limiting embodiments, the subject is one that has suffered a stroke, and has developed a clot, and the therapeutic combination comprising mammalian exosomes, tPA, and/or thrombectomy is administered to the subject after the development of clot in order to ameliorate and/or relieve the symptoms via dissolution of the clot. In another embodiment, the subject is one that has suffered a stroke, and has not developed a clot and the therapeutic combination comprising mammalian exosomes and/or microvesicles, tPA, and/or thrombectomy is administered to the subject to prevent the development of fibrin cross-links. Accordingly, therapeutic combination (i.e., mammalian exosomes, tPA, and/or thrombectomy) can be administered to the subject prior to the event occurring; concurrently with the event; and/or after the event occurs but before the development of clot formation.

tPA has a short therapeutic window of about 2-4 hours in which it may be successfully administered; otherwise, the risk of side effects and/or lack of efficacy outweigh its benefits. In some non-limiting embodiments, the methods of the present disclosure may be practiced on a subject that has suffered a stroke and has been examined and treated by a medical professional after a period ranging from about 10 minutes to about 24 hours from initial diagnosis or detection of symptoms of the onset of the stroke, using a therapeutically effective combination comprising mammalian exosomes and/or microvesicles and tPA and/or thrombectomy. Thus, the therapeutic combination (i.e., mammalian exosomes and/or microvesicles, tPA, and/or thrombectomy) may be administered to the subject to extend the available therapeutic window (approximately within 2-4 hours in humans) in which to administer tPA treatment to about 0.5-12 hours or to about 0.5-9 hours, or to about 0.5-9 hours.

In addition to disrupting the clot via the cleaving of fibrin, tPA also reduces the size of the clot. In some non-limiting embodiments, the subject is one that has suffered a stroke, and has developed a clot, and the therapeutic combination comprising mammalian exosomes and/or microvesicles and tPA and/or thrombectomy is administered to the subject after the development of clot and/or symptoms of stroke or cerebrovascular injury in order to ameliorate and/or relieve the symptoms reducing the size of the clot, and restoring blood, oxygen, and/or nutrients to the ischemic area. In another embodiment, the subject is one that has suffered a stroke, and has not yet developed a clot and the therapeutic combination comprising mammalian exosomes and/or microvesicles, and tPA and/or thrombectomy is administered to the subject to prevent the development of the clot, or prevent a clot from becoming established, and if a clot is established, then preventing it from increasing in size.

The blood-brain barrier (BBB) describes the highly regulated vasculature that delivers blood, oxygen, and nutrients to the brain and central nervous system. The vasculature that makes up the BBB possess unique properties allows regulated movement of molecules, ions, and cells; see Daneman and Prat, “The Blood-Brain Barrier,” Cold Spring Harb Perspect Biol. 2015 January; 7(1). Disruption and/or leakage of the blood-brain-barrier can have deleterious effects on brain function. In some non-limiting embodiments, the subject is one that has suffered a stroke, and has incurred a disruption of the BBB, and the therapeutic combination (i.e., mammalian exosomes, tPA, and/or thrombectomy) is administered to the subject after the disruption of the BBB and/or after symptoms of stroke or cerebrovascular injury present, in order to ameliorate and/or relieve the disruption and/or leakage of the BBB. In another embodiment, the subject is one that has suffered a stroke, and has not yet developed a disruption of the BBB, and the therapeutic combination is administered to the subject to prevent the disruption of the BBB. Accordingly, therapeutic combination (i.e., mammalian exosomes and/or microvesicles, tPA, and/or thrombectomy) can be administered to the subject prior to the event occurring; concurrently with the event; and/or after the event occurs but before the disruption of the BBB.

Efficacy and/or success following the administration of the therapeutic combination (i.e., mammalian exosomes and/or microvesicles, tPA, and/or thrombectomy) can be evaluated based on the amelioration, mitigation, reduction, and/or complete elimination of one or more of the symptoms enumerated above. Efficacy and/or success in the prevention (i.e., prophylaxis regarding the occurrence or recurrence of a particular condition, disease, or disorder, in an individual), can be evaluated based on whether an individual who may be predisposed to, susceptible to a particular condition, disease, or disorder, or at risk of developing such a condition, disease, or disorder, eventually develops the disease, disorder or condition. For cases of ischemic stroke, recanalization, revascularization, and reperfusion of occluded vessels, along with the National Institute of Health Stroke Scale/Score (NIHSS), are key variables in assessing the success of a given treatment modality.

In some embodiments, the effect of the present treatment method can be assessed based on thrombolysis in cerebral ischemia (TICI), and/or thrombolysis in myocardial ischemia (TIMI). Alternatively, in some embodiments, a modified thrombolysis in cerebral ischemia (mTICI) scale may be used, which scores angiographic criteria based on 0 (no perfusion); 1 (minimal flow past the occlusion with little to no perfusion); 2a (antegrade partial perfusion of less than half of the downstream ischemic territory); 2b (antegrade partial perfusion of half or greater of the downstream ischemic territory); and 3 (antegrade complete perfusion of the downstream ischemic territory). In other embodiments, success of the treatment method and, for example, a thrombectomy procedure, can be evaluated based on complete evacuation of the thrombus (i.e., brisk reflux of blood), and repeat angiogram findings (see Zaidat et al., Revascularization grading in endovascular acute ischemic stroke therapy, Neurology. 2012 Sep. 25; 79(13 Suppl 1): S110-S116).

In some embodiments, the success of the therapeutic combination (i.e., mammalian exosomes and/or microvesicles, tPA, and/or thrombectomy) can be gauged based on the reduction of clot size by at least 10%-50%, for example, at least 30% after administration of one or more courses of treatment.

As used herein, a “measurable thrombolytic effect” refers to the ability to assess increased proteolysis of fibrin in a clot, for example using biomarkers of fibrin cleavage; reduction of the clot size, for example, a reduction of at least 10%-50%, for example, at least 30% as indicated by angiogram, or other imaging techniques known to those in the art; increases the rate and extent of vessel recanalization, as indicated by angiography, and/or Doppler imaging; increases microvascular reperfusion without increased brain hemorrhage, as indicated by improving neurological function, and physical presentations; reduction of leakage of the blood-brain-barrier; and attenuation of infarct expansion.

Exclusion criteria for thrombolysis include historical criteria such as stroke or head trauma in the previous three months; intracranial neoplasm, arteriovenous malformation, or aneurysm; recent intracranial or intraspinal surgery; previous intracranial hemorrhage; and/or arterial puncture at a non-compressible site in the previous seven days (see Jauch E C, et al. Guidelines for the early management of patients with ischemic stroke: a guideline for healthcare professionals from the American Heart Association/American Stroke Association. Stroke 2013; 44:870). Clinical exclusion criteria for thrombolytic therapy include symptoms suggestive of subarachnoid hemorrhage; serum glucose <50 mg/dL (<2.8 mmol/L); persistent blood pressure elevation (systolic ≥185 mmHg or diastolic ≥110 mmHg); active internal bleeding; and/or acute bleeding diathesis (see Jauch et al.). Hematologic exclusion criteria include platelet count <100,000/mm³; heparin use within 48 hours and an abnormally elevated aPTT; current anticoagulant use with an INR >1.7 or PT >15 seconds; and/or current use of a direct thrombin inhibitor or direct factor Xa inhibitor with evidence of anticoagulant effect by laboratory tests such as aPTT, INR, ECT, TT, or appropriate factor Xa activity assays. Furthermore, evidence of hemorrhage and/or extensive regions of obvious hypodensity consistent with irreversible injury on a head CT scan are also contraindications for thrombolytic therapy.

In some embodiments, a subject diagnosed as having suffered a stroke and is not eligible for thrombolysis using tPA may be treated by receiving a therapeutic combination comprising mammalian exosomes and/or microvesicles and thrombectomy. In various embodiments, the tPA ineligible stroke patient is administered a therapeutically effective dose of mammalian exosomes and/or microvesicles prior to the thrombectomy procedure. In some embodiments, the tPA ineligible stroke patient is administered a therapeutically effective dose of mammalian exosomes and/or microvesicles prior to the thrombectomy procedure, and after the thrombectomy procedure.

In some aspects of the methods, kits and compositions of the present disclosure, a mammalian exosome can comprise miR-19a, miR-21 microRNA, miR-146a or any combination thereof. In addition to miR-19a, miR-21, miR-146a or any combination thereof, a mammalian exosome can further comprise any other number of proteins (e.g. Alix and/or CD63), growth factors, microRNAs, siRNAs and mRNAs. In a non-limiting example, a mammalian exosome can comprise antibodies to cell surface proteins that specifically target the exosome to specific tissues of interest.

As used herein, the term “microRNAs” (miRNAs or miRs), is used to describe short RNA molecules (20-24 nt) that can be involved in the regulation of gene expression via their effect on mRNA stability and translation of the target mRNA. miRNAs are sometimes transcribed as longer primary mRNA transcripts called a pre-miR. The pre-miR is subsequently processed to yield a mature miR. Thus as used herein, miR-19a can also refer to pre-miR-19a, miR-21 can also refer to pre-miR-21 and miR-146a can also refer to pre-miR-146a.

As used herein, miR-19a can also refer to pre-miR-19a. In some aspects, pre-miR-19a can comprise the nucleotide (RNA) sequence:

(SEQ ID NO: 8)
GCAGUCCUCUGUUAGUUUUGCAUAGUUGCACUACAAGAAGAAUGUAGUUG
UGCAAAUCUAUGCAAAACUGAUGGUGGCCUGC,

which is encoded by the pre-miR-19a DNA sequence, which can comprise the nucleotide (DNA) sequence:

(SEQ ID NO: 9)
GCAGTCCTCTGTTAGTTTTGCATAGTTGCACTACAAGAAGAATGTAGTTG
TGCAAATCTATGCAAAACTGATGGTGGCCTGC.

As used herein, miR-19a can also refer to mature miR-19a. in some aspects, mature miR-19a can comprise the nucleotide (RNA) sequence:

| (SEQ ID NO: 10) | |
|---|---|
| | AGUUUUGCAUAGUUGCACUACA, |

which is encoded by the mature miR-19a DNA sequence, which can comprise the nucleotide (DNA) sequence:

| (SEQ ID NO: 11) | |
|---|---|
| | AGTTTTGCATAGTTGCACTACA. |

As used herein, miR-21 can refer to pre-miR-21. In some aspects, pre-miR-21 can comprise the nucleotide (RNA) sequence:

(SEQ ID NO: 12)
UGUCGGGUAGCUUAUCAGACUGAUGUUGACUGUUGAAUCUCAUGGCAACA
CCAGUCGAUGGGCUGUCUGACA,

which is encoded by the pre-miR-21 DNA sequence, which can comprise the nucleotide (DNA) sequence:

(SEQ ID NO: 13)
TGTCGGGTAGCTTATCAGACTGATGTTGACTGTTGAATCTCATGGCAACA
CCAGTCGATGGGCTGTCTGACA.

As used herein, miR-21 can also refer to mature miR-21. in some aspects, mature miR-21 can comprise the nucleotide (RNA) sequence:

| (SEQ ID NO: 14) | |
|---|---|
| | UAGCUUAUCAGACUGAUGUUGA, |

which is encoded by the mature miR-21 DNA sequence, which can comprise the nucleotide (DNA) sequence:

| (SEQ ID NO: 15) | |
|---|---|
| | TAGCTTATCAGACTGATGTTGA. |

As used herein, miR-146a can also refer to pre-miR-146a. In some aspects, pre-miR-146a can comprise the nucleotide (RNA) sequence:

(SEQ ID NO: 16)
CCGAUGUGUAUCCUCAGCUUUGAGAACUGAAUUCCAUGGGUUGUGUCAGU
GUCAGACCUCUGAAAUUCAGUUCUUCAGCUGGGAUAUCUCUGUCAUCGU,

which is encoded by the pre-miR-146a DNA sequence, which can comprise the nucleotide (DNA) sequence:

(SEQ ID NO: 17)
CCGATGTGTATCCTCAGCTTTGAGAACTGAATTCCATGGGTTGTGTCAGT
GTCAGACCTCTGAAATTCAGTTCTTCAGCTGGGATATCTCTGTCATCGT.

As used herein, miR-146a can also refer to mature miR-146a. in some aspects, mature miR-146a can comprise the nucleotide (RNA) sequence:

| (SEQ ID NO: 18) | |
|---|---|
| | UGAGAACUGAAUUCCAUGGGUU, |

which is encoded by the mature miR-146a DNA sequence, which can comprise the nucleotide (DNA) sequence:

| (SEQ ID NO: 19) | |
|---|---|
| | TGAGAACTGAATTCCATGGGTT. |

In some aspects of the methods, kits and compositions of the present disclosure, a mammalian exosomes can be enriched in miR-19a, miR-21, miR-146a, or any combination thereof. A mammalian exosome is said to be enriched for particular microRNA when the concentration of the particular microRNA in the exosome is greater than the concentration of the particular microRNA in a control or naïve exosome. Alternatively, a mammalian exosome can be non-enriched for miR-19a, miR-21, miR-146a, or any combination thereof.

In some aspects of the methods, kits and compositions of the present disclosure, a mammalian exosome can be a mammalian cell-derived exosome that initially contained little to no miR-19a, miR-21, miR-146a or any combination thereof, but which was transformed with miR-19a coding nucleic acids, miR-21 coding nucleic acids, miR-146a coding nucleic acids or any combination thereof, thereby enriching the exosome for miR-19a, miR-21, miR-146a or any combination thereof. In this aspect, coding nucleic acids can include, but are not limited to, plasmids which contain polynucleotides operable to encode miR-19a, miR-21, miR-146a or any combination thereof.

In some aspects of the preceding methods, a mammalian exosome can be an exosome that is not specifically transformed recombinantly (non-naturally) with an exogenous nucleic acid.

In aspects of the preceding methods, the mammalian exosomes, tPA or combination thereof can be administered to the subject using an administration method known to those of ordinary skill in the art, including, but not limited to, parenteral, intravenous, intraarterial, subcutaneous, intramuscular, intraperitoneal, stereotactic, intranasal, mucosal, intravitreal, intrastriatal, or intrathecal administration. Administration methods can be continuous, chronic, short, intermittent or any combination thereof. In the aspects in which a combination of mammalian exosomes and tPA are administered to the subject, the mammalian exosomes and tPA can be administered using the same method or different methods.

Administration of mammalian exosomes and tPA can occur concomitantly, or sequentially, for example, with mammalian exosomes being administered first and tPA being administered after; with tPA being administered followed by the administration of mammalian exosomes; or tPA and mammalian exosomes may be administered at the same time. Thus, in some aspects, a therapeutically effective dose of mammalian exosomes will be administered before, after, and/or at the same time as tPA; the tPA being administered at a therapeutically effective dose, or at a suboptimal dose.

In some aspects, without limitation, the preceding methods may utilize compositions containing mammalian exosomes and/or microvesicles, optionally in combination with tPA. In some aspects, the compositions of the present methods are administered separately. In other aspects, an illustrative composition comprises mammalian cell derived exosomes and/or microvesicles and tPA in a single composition. In some aspects, the mammalian exosomes include exosomes and/or microvesicles derived from an exosome producing cell.

In some aspects, the methods of the present disclosure may be practiced on a subject between about 10 minutes to about 6 hours after the occurrence of stroke. In some aspects, the methods of the present disclosure may be practiced on a subject between about 10 minutes to about 12 hours, or about 10 minutes to about 18 hours, or about 10 minutes to about 24 hours, or about 10 minutes to about 30 hours, or about 10 minutes to about 36 hours, or about 10 minutes to about 42 hours, or about 10 minutes to about 48 hours, or about 10 minutes to about 72 hours, or about 10 minutes to about 96 hours, or about 10 minutes to about 120 hours, or about 10 minutes to about 144 hours, or about 10 minutes to about 168 hours after the occurrence of a stroke.

In some aspects, the methods of the present disclosure may be practiced on a subject about 0.5 hours, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, or about 3 days, or about 4 days, or about 5 days, or about 6 days, or about 7 days, or about 8 days, or about 9 days, or about 10 days, or about 11 days, or about 12 days, or about 13 days, or about 14 days, or about 3 weeks, or about 4 weeks after occurrence of a stroke.

In some aspects, a combination of a therapeutically effective amount of exosomes and a therapeutically effective amount of tPA can be administered to a subject about 0.5 hours, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, or about 3 days, or about 4 days, or about 5 days, or about 6 days, or about 7 days, or about 8 days, or about 9 days, or about 10 days, or about 11 days, or about 12 days, or about 13 days, or about 14 days, or about 3 weeks, or about 4 weeks after occurrence of a stroke.

In some aspects, a therapeutically effective amount of exosomes can be administered to a subject about 0.5 hours, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, or about 3 days, or about 4 days, or about 5 days, or about 6 days, or about 7 days, or about 8 days, or about 9 days, or about 10 days, or about 11 days, or about 12 days, or about 13 days, or about 14 days, or about 3 weeks, or about 4 weeks after occurrence of a stroke.

In some aspects, a therapeutically effective amount of tPA can be administered to a subject about 0.5 hours, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, or about 3 days, or about 4 days, or about 5 days, or about 6 days, or about 7 days, or about 8 days, or about 9 days, or about 10 days, or about 11 days, or about 12 days, or about 13 days, or about 14 days, or about 3 weeks, or about 4 weeks after occurrence of a stroke.

In some aspects, a thrombectomy can be performed on a subject about 0.5 hours, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, or about 3 days, or about 4 days, or about 5 days, or about 6 days, or about 7 days, or about 8 days, or about 9 days, or about 10 days, or about 11 days, or about 12 days, or about 13 days, or about 14 days, or about 3 weeks, or about 4 weeks after occurrence of a stroke.

In some aspects of the methods of the present disclosure, treating or preventing can comprise reducing a clot and/or thrombus by at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 100%.

Increasing proteolysis of fibrin in a clot and/or thrombus can comprise increasing proteolysis by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%, or by at least about 200%, or by at least about 300%, or by at least about 400%, or by at least about 500%, or by at least about 600%, or by at least about 700%, or by at least about 800%, or by at least about 900%, or by at least about 1000%.

Increasing the rate and extent of vessel recanalization can comprise increasing the rate by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%, or by at least about 200%, or by at least about 300%, or by at least about 400%, or by at least about 500%, or by at least about 600%, or by at least about 700%, or by at least about 800%, or by at least about 900%, or by at least about 1000%.

Increasing the microvascular reperfusion without increasing infarct expansion can comprise increasing microvascular reperfusion by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%, or by at least about 200%, or by at least about 300%, or by at least about 400%, or by at least about 500%, or by at least about 600%, or by at least about 700%, or by at least about 800%, or by at least about 900%, or by at least about 1000%.

Reducing leakage of the blood-brain-barrier can comprise reducing leakage by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%.

Reducing the size of a clot or thrombus can comprise reducing the size by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%.

Reducing the expansion of an ischemic core can comprise reducing the expansion by at least about 10%, or by at least about 15%, or by at least about 20%, or by at least about 25%, or by at least about 30%, or by at least about 35%, or by at least about 40%, or by at least about 45%, or by at least about 50%, or by at least about 55%, or by at least about 60%, or by at least about 65%, or by at least about 70%, or by at least about 75%, or by at least about 80%, or by at least about 85%, or by at least about 90%, or by at least about 95%, or by at least about 100%.

Kits

In some embodiments, the present disclosure provides kits for the treatment and prevention of stroke and cerebrovascular injury resulting from stroke, for example ischemic stroke. In some embodiments, the kit of the present disclosure comprises mammalian exosomes and/or microvesicles. In some embodiments, the mammalian exosomes and/or microvesicles are derived from for example, stem cells, mesenchymal stromal cells, umbilical cord cells, endothelial cells, for example, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes, of any of the foregoing cells cultured in vitro. In various embodiments, the kits of the present disclosure contain mammalian exosomes and/or microvesicles derived from the above referenced mammalian cells, and wherein the exosomes and/or microvesicles contain one or more of miR-19a, miR-21, and miR-146a microRNA. The kit of the present disclosure can include one or more doses of mammalian exosomes and/or microvesicles in combination with a therapeutic dose of tPA, in the same or separate compositions. In some embodiments, the kit of the present disclosure. The kit of the present disclosure can include one or more doses of mammalian exosomes and/or microvesicles in combination with a therapeutic dose of tPA, in the same or separate compositions. In some embodiments, the kit of the present disclosure can include one or more doses of mammalian exosomes and/or microvesicles in combination with a surgical device useful in the performance of a thrombectomy procedure. In various embodiments, the kit of the present disclosure also includes a package insert comprising instructions for using the mammalian exosomes and/or microvesicles and tPA and/or thrombectomy device to treat or a cerebrovascular injury. In some embodiments, the cerebrovascular injury is neuronal damage, residual clot persistence, microvascular hypoperfusion, blood-brain-barrier leakage, or ischemic lesion expansion. In some embodiments, the cerebrovascular injury is the presentation of symptoms consistent with is neuronal damage, residual clot persistence, microvascular hypoperfusion, blood-brain-barrier leakage, or ischemic lesion expansion.

While some embodiments have been particularly shown and described with reference to the foregoing preferred and alternative embodiments, it should be understood by those skilled in the art that various alternatives to the embodiments described herein may be employed in practicing the invention without departing from the spirit and scope of the invention as defined in the following claims. It is intended that the following claims define the scope of the invention and that the methods, systems, and compositions within the scope of these claims and their equivalents be covered thereby. This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### EXAMPLES

The following examples of some embodiments are provided without limiting the invention to only those embodiments described herein and without waiving or disclaiming any embodiments or subject matter.

### Example 1. Exosomes Derived from Cerebral Endothelial Cells (CEC-Exosomes) and Acute Stroke

Exosomes are endosomal origin membranous nanovesicles that mediate intercellular communication by transferring cargo proteins, lipids, and genomic materials including miRNAs between source and target cells. The inventors' laboratory was the first to demonstrate that exosomes derived from mesenchymal stromal cells (MSCs) given to rats 24 h after middle cerebral artery occlusion (MCAO) substantially promote brain remodeling processes and stroke recovery by transferring exosome miRNAs to brain parenchymal cells. MCAO is a stroke with large artery occlusion. However, the effect of CEC exosomes on acute stroke in particular on ischemic stroke with large artery occlusion remains unknown. The in vitro preliminary data show that compared to MSC-exosomes, CEC-exosomes exert a more robust effect on reducing blood brain barrier (BBB) leakage. CEC-exosomes are enriched with molecules including proteins that regulate BBB function. Endothelial derived exosomes have promising therapeutic potential. In various embodiments exemplified herein, CEC-exosomes were used experimentally in the examples described herein, but other mammalian exosomes are believed to function in a similar manner, and for which CEC-exosomes are used as a representative source of mammalian exosomes for purposes of convenience to demonstrate the therapeutic effects of administering mammalian exosomes with tPA.

Using a rat model of embolic MCAO, which mimics patients with a large artery occlusion, the experimental data gathered have demonstrated that tPA given 4 h after MCAO does not have any therapeutic effect in the experimental animal models. However, the experimental preliminary data provided herein unexpectedly now shows that compared to monotherapy with tPA, intravenous (IV) administration of CEC-exosomes in combination with tPA to rats subjected to 4 h of embolic MCAO significantly reduced ischemic lesion and improved neurological outcome by facilitating recanalization of the occluded MCA and augmenting microvascular reperfusion without increasing brain hemorrhage. Moreover, CEC-exosomes either delivered intravenously (IV) or intraarterially (IA) were shown to cross the blood brain barrier (BBB). These exemplified data herein, demonstrate that CEC-exosomes amplify tPA-induced thrombolysis. The data provided herein provides the unexpected results to show that CEC-exosome therapy can be used as an adjunctive treatment to enhance tPA and thrombectomy treatment of acute ischemic stroke.

Exosomes, cerebral vascular injury, and secondary thrombosis: Large artery occlusion leads to dynamic ongoing infarct expansion. Injured cerebral endothelial cells amplify thrombosis by recruiting leukocytes and platelets and promote BBB leakage, leading to neurovascular damage. Preclinical data demonstrate that occlusion of a large artery by a clot triggers secondary thrombotic development at the occluded site and at the downstream of cerebral microvessels, which contributes to microvascular hypoperfusion, disruption of the BBB and irreversible neuronal damage. Moreover, in addition to thrombolysis, tPA induces reperfusion injury by activating pro-thrombotic and BBB disruptive genes, which exacerbate neurovascular damage and cause brain hemorrhage. Although many factors including excitotoxicity, oxidative stress, and activation of platelets and leukocytes have been implicated to induce thrombosis, mechanisms underlying formation of secondary thrombosis remain to be investigated.

Large artery occlusion leads to dynamic ongoing infarct expansion. Among many factors that contribute to infarct expansion including excitotoxicity and oxidative stress, microvascular thrombosis-related no-flow is a key factor for development of infarction. Preclinical data demonstrate that occlusion of a large artery by a clot triggers secondary thrombotic development at the occluded site and at the downstream cerebral microvessels. This ongoing process occurs over many hours, and coincides spatially and temporally with reduction of cerebral tissue perfusion, disruption of the blood brain barrier (BBB) and irreversible neuronal damage, and eventually leads maturation of infarct expansion. Thus, inhibition of secondary thrombotic formation in downstream cerebral microvessels prior to thrombolysis and thrombectomy prevents infarct expansion, and will increase the numbers of patients who would be eligible to receive tPA and thrombectomy. In addition, suppressing secondary thrombosis after tPA/thrombectomy augments tissue reperfusion, leading to better functional outcome.

Without wishing to be bound by theory, the principle of thrombolytic therapy is to dissolve the fibrin contained in a clot to re-establish blood flow. In stroke patients, rapid recanalization after thrombolytic therapy and/or thrombectomy is essential to reduce infarction and to achieve a favorable clinical outcome compared with persistent occlusion of the artery. However, the thrombolytic effects of tPA and thrombectomy are far from optimal. Only one third of patients with large artery occlusion treated with tPA within 4.5 h of stroke onset achieve reperfusion. Moreover, in addition to thrombolysis, tPA induces reperfusion injury by activating pro-thrombotic and BBB disruption genes, which exacerbates neurovascular damage and causes brain hemorrhage. Recanalization of the occluded large artery by the thrombectomy only leads to 71% of patients achieving improved and often incomplete tissue reperfusion. To amplify the therapeutic effect of thrombectomy and tPA-induced thrombolysis, thus, new therapies are urgently required to minimize neurovascular damage by suppressing development of secondary thrombosis, reperfusion injury, BBB leakage and ischemic cell damage.

Neutrophil extracellular traps (NETs) mediate thrombotic formation and have recently been detected in thrombi retrieved from patients with acute ischemic stroke. NETs containing DNA and histones are more resistant to tPA and the addition of DNase 1 increases the efficacy of tPA-mediated thrombolysis. When it blocks a vessel, the clot injures cerebral endothelial cells. P-selectin released by injured endothelial cells initiates NET formation that is followed by recruited platelets. High mobility group protein B1 (HMGB1) derived from platelets further promotes formation of NETs. Neutrophils by promoting platelet thromboxane A2 induce endothelial cell expression of intercellular adhesion molecule 1 (ICAM1), which strengthens neutrophil interactions with the endothelium. These processes are mediated by toll-like receptor (TLR) signaling.

Using exosomes derived from thrombectomy-retrieved clots lodged in the large cerebral artery of patients with acute stroke, the inventor's in vitro data show that clot-derived exosomes induced BBB leakage by triggering healthy human cerebral endothelial cells (CECs) to upregulate a set of proteins including ICAM1, P-selectin, HMGB1, TLR2 and TLR4, which are involved in formation of NETs. In addition, these proteins cause vascular injury and thrombogenicity. In the past, stroke thrombi were not available for study. The inventor's data for the first time demonstrate that patient-clot-derived exosomes induce dysfunction of healthy CECs, suggesting that clot generated exosomes stimulate CECs to upregulate those proteins that promote formation of NETs in thrombi, secondary thrombosis in downstream microvessels, and BBB impairment. These data provide new insights into molecular mechanisms underlying secondary thrombotic formation. Importantly, data presented herein has led to the idea that, in one aspect, exosomes derived from healthy endothelial cells (CEC-exos) diminished clot-exosome-upregulated proteins and BBB leakage, and CEC-exo thereby enhance the therapeutic efficacy of tPA thrombolysis/thrombectomy and BBB integrity. Mature miRNA binds either to target sites in coding regions of target mRNAs for destabilization or to the 3′-untranslated regions (UTRs) leading to translational repression. miR-146a is an miRNA that targets TLR signaling. Rodent and human cerebral endothelial cells express miR-146a. The TLR signaling pathway mediates cerebral endothelial cell function and activation of the TLR signal triggers releasing NF-KB signals related pro-inflammatory cytokines, leading to disruption of BBB integrity. Upregulation of miR-146a reduces cerebral microvascular thrombosis and BBB leakage. MiR-21 targets TLR4 and HMGB1. The inventors have previously shown that increased miR-21 reduces ischemic neuronal damage. Tissue factor (TF) catalyzes coagulation process by binding to activated coagulation factor VII (VIIa), leading to thrombin generation, fibrin deposition, and thrombus formation. TF in microvessels catalyzes intravascular fibrin formation that results in leakage of albumin and large blood molecules such as fibrinogen from the BBB to parenchyma. miR-19 exerts anti-thrombotic effect by suppressing genes of TF and plasminogen activator inhibitor 1 (PAI1). Reduction of miR-19a levels has been detected in blood samples of patients with acute stroke. MiR-19, -21, and -146a are well conserved between human and rodent. The inventors' preliminary data show that patient-clot-derived exosomes (otherwise known as “clot-injured CEC exosomes”) substantially downregulated a set of miRNAs including miR-19a, miR-21, and miR-146a in healthy cerebral endothelial cells, which is reversed by healthy CEC-exosomes. Moreover, bioinformatics analysis revealed that this set of RNAs forms a network with aforementioned proteins. In parallel to these in vitro human data, the in vivo animal preliminary data provided herewith show that CEC-exosomes in combination tPA robustly increases levels of miR-21 and miR-146a in ischemic cerebral endothelial cells, which are associated with substantial reduction of ICAM1, TLR4, and activated NF-κB. Moreover, alteration of the miRNAs and proteins is highly associated with reduction of thrombosis and vascular damage revealed by transmission electron microscopy (TEM) and immunohistological analysis. These patient and animal data suggest that this network of miRNAs/proteins in cerebral endothelial cells likely mediates stroke-induced neurovascular damage. CEC-exosomes administered to the stroke patient (for example, via intravenous (IV) administration), are detected in endothelial cells of cerebral blood vessels. Without wishing to be bound by any particular theory, the examples provided herein suggest that CEC-exosomes derived from healthy CECs act on cerebral endothelial cells to reduce vascular injury and formation of secondary thrombosis by delivering CEC-exosome cargo miR-19a, miR-21, and miR-146a to repress the network of miRNAs/proteins that promote vascular injury and thrombogenicity. This data presented herein provides some evidence that underlies molecular mechanisms for the therapeutic effect of CEC-exosomes on enhancement of thrombolysis and reduction of neurovascular damage.

Without wishing to be bound by theory, the scientific premises are that CEC-exosomes attenuate ongoing infarct expansion and that CEC-exosomes in combination with tPA and/or thrombectomy are more effective in improving neurological outcome than monotherapy of tPA or thrombectomy in acute ischemic stroke with large artery occlusion. Currently only ˜10% and 7-15% of ischemic stroke patients receive tPA and endovascular interventions, respectively. The inventors expect that successful administration of the compositions of the present disclosure may not only increase the number of patients with ischemic stroke who are eligible for these interventions, but will also provide an effective and safe exosome-based therapy to enhance reperfusion, leading to maximized improvement of neurological outcomes.

The inventors have pioneered the use of subacute delayed exosomal therapy for stroke to promote brain remodeling. However, the present disclosure provides compositions, methods and systems for a mammalian cell exosome population (for example, a cerebral endothelial exosome)-based therapy for acute ischemic stroke with large artery occlusion. This approach is highly novel and the first to consider treatment of acute ischemic stroke with exosomes, for example, CEC-exosomes, to enhance tPA-mediated thrombolysis and on microvascular perfusion and neurovascular damage induced by transient MCAO, which mimics thrombectomy. The preliminary data provided herewith demonstrate that CEC-exosomes augment tPA-mediated thrombolysis. The use of CEC-exosomes for clinical application as an adjuvant therapy in combination with tPA and thrombectomy is provided in various methods described herein for the treatment of patients with large artery occlusion as commonly occurs in stroke.

Preliminary data from patient clot-injured CEC exosomes and animal experiments show that healthy CEC-exosomes target cerebral endothelial cells to reduce vascular injury, leading to suppression of secondary thrombosis via suppression of the network of miRNAs/proteins that promote vascular injury and thrombogenicity. These data provide a novel mechanism underlying the therapeutic effect of CEC-exosomes on enhancement of thrombolysis and reduction of neurovascular damage.

### Example 2. CEC-Exosome Therapy as an Adjunctive Treatment in Combination with tPA and Thrombectomy Treatment Enhances tPA and Thrombectomy Treatment in Aged Rats after Large Artery Occlusion

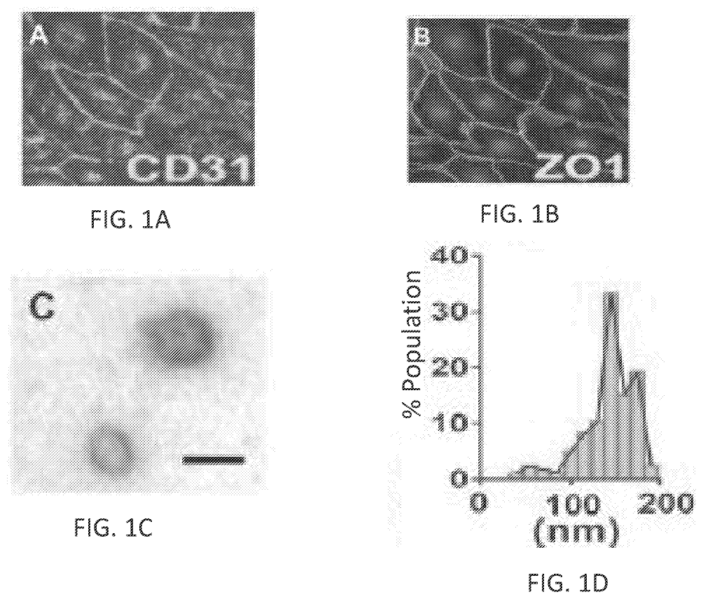

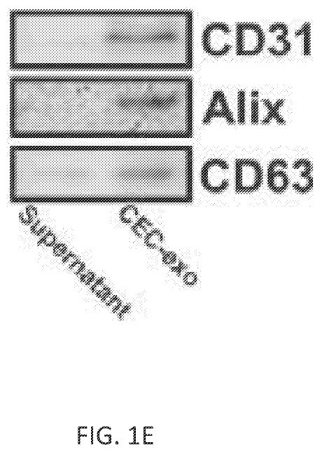

CEC-exosomes vs MSC-exosomes. Using ultracentrifugation, the inventors have isolated exosomes from the supernatant of cultured primary cerebral endothelial cells and then characterized these CEC-exosomes by means of well-established standard methods. (FIG. 1). The inventors found that these exosomes exhibited characteristic doughnut morphology, mean diameter ˜140 nm, and tetraspanin protein CD63 and endosome membrane protein Alix (See results provided in FIG. 1). Previous published data demonstrated that exosomes derived from MSCs (MSC-exosomes) given 24 h after MCAO promote brain remodeling and stroke recovery. Using an in vitro assay of BBB leakage induced by patient-clot-derived exosomes, the inventors compared the effect of CEC-exosomes with MSC-exosomes on BBB leakage. The inventors found that CEC-exosomes suppressed BBB leakage by more than 75% (28%±2% vs 100% in control, n=5), whereas MSC-exosomes decreased BBB leakage by only ˜12% (88%±5% vs 100% in control, n=5), which was significantly (p<0.05) less efficient than CEC-exosomes in suppressing BBB leakage. The inventors thus selected CEC-exosomes in the performance of subsequent experiments.

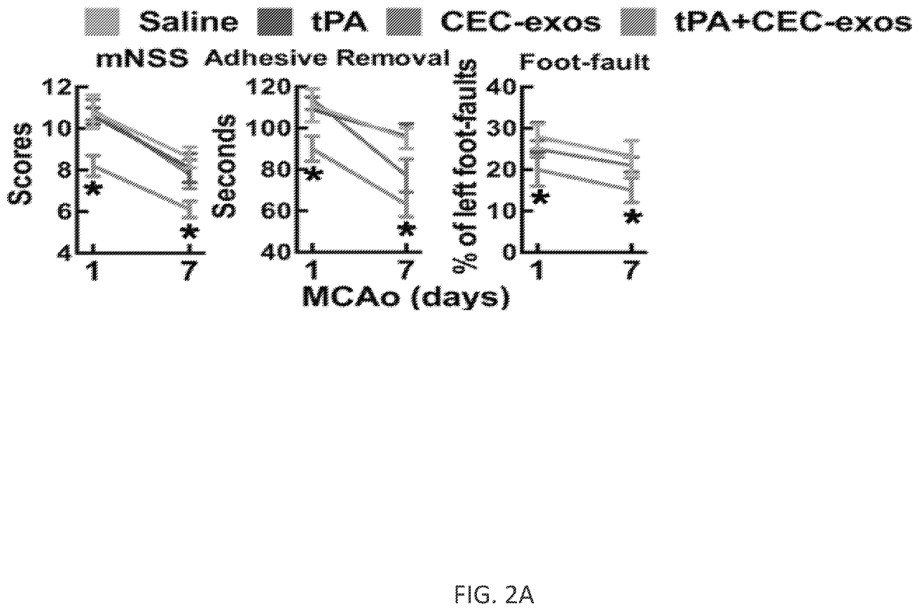

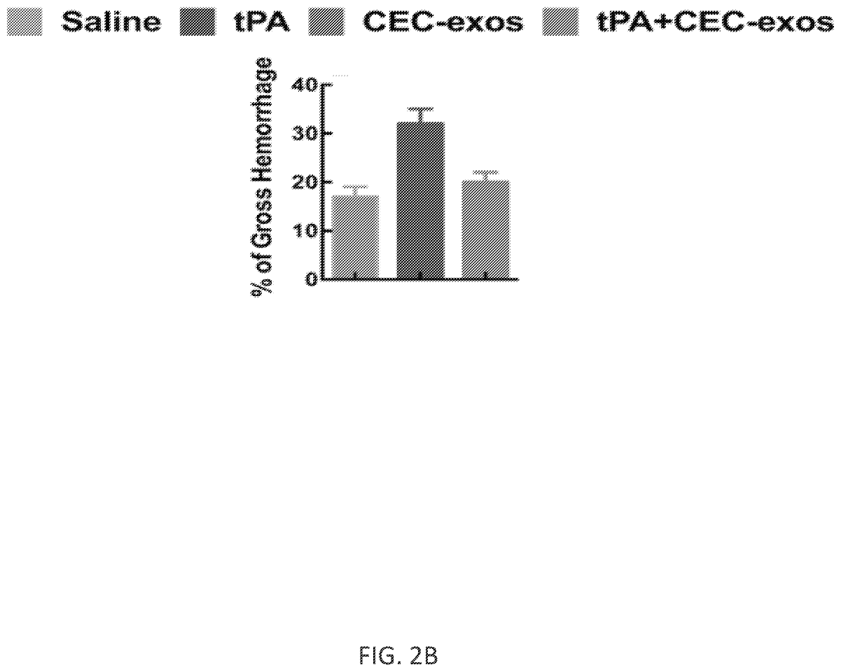

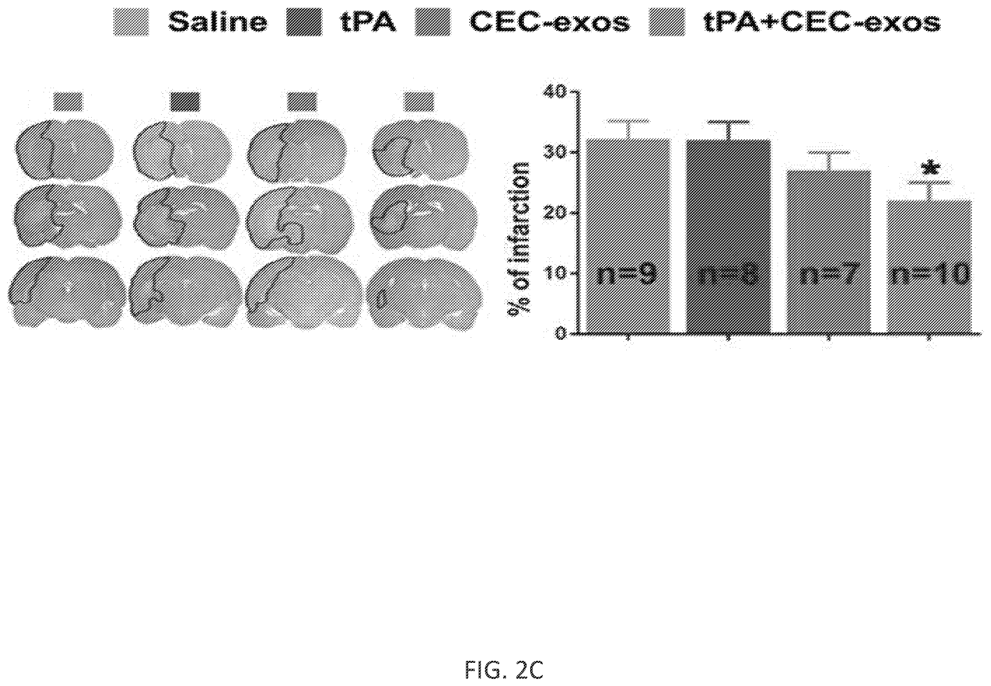

CEC-exosomes in combination with tPA significantly reduce infarct volume and improve neurological outcomes. Using a model of embolic MCAO that mimics patients with ischemic stroke of large artery occlusion, the inventors have demonstrated that tPA administered 4 h after MCAO (equivalent to two hours post therapeutic window for rats and equivalent to administration of tPA 6 hours after stroke in humans) the therapeutic window in humans does not have a therapeutic effect on acute stroke. To examine whether CEC-exos treatment enhances the therapeutic effect of tPA, CEC-exos (1×10¹¹ particles/injection) were administered via a tail vein to young adult male rats at 4 h and 24 h after embolic middle cerebral artery occlusion (eMCAO), while tPA (10 mg/kg, iv) was given 4 h after eMCAO. All rats exhibited severe neurological deficits with a mean score of 3 assayed on the Longa five point scale prior to the treatment at 2 h after eMCAO, Compared to the saline treatment, the monotherapy of tPA did not significantly improve neurological deficits measured with an array of well-established behavioral tests that detect sensorimotor deficits, although some spontaneous recovery occurred (FIG. 2A). However, CEC-exos combination with tPA significantly reduced neurological deficits at 1 day and 7 days (7 d) after eMCAO (FIG. 2A). The combination treatment did not significantly increase gross brain hemorrhage (20% vs 17% in saline and 32% in tPA, FIG. 2B). Histopathological analysis of brains from the rats sacrificed 7 d after eMCAO revealed that the combination treatment significantly reduced infarct volume by ˜40% (FIG. 2C). These data indicate that CEC-exos treatment in combination with tPA has a therapeutic effect for acute ischemic stroke induced by large artery occlusion, and has a synergistic effect on tPA monotherapy. Moreover, endovascular therapy for acute ischemic stroke is currently available only at highly specialized stroke centers. tPA treatment is still the standard care for acute ischemic stroke.

The experiments provided herein also seek to investigate the effect of the combination treatment with CEC-exosomes and tPA in aged male and female rats subjected to embolic MCAO.

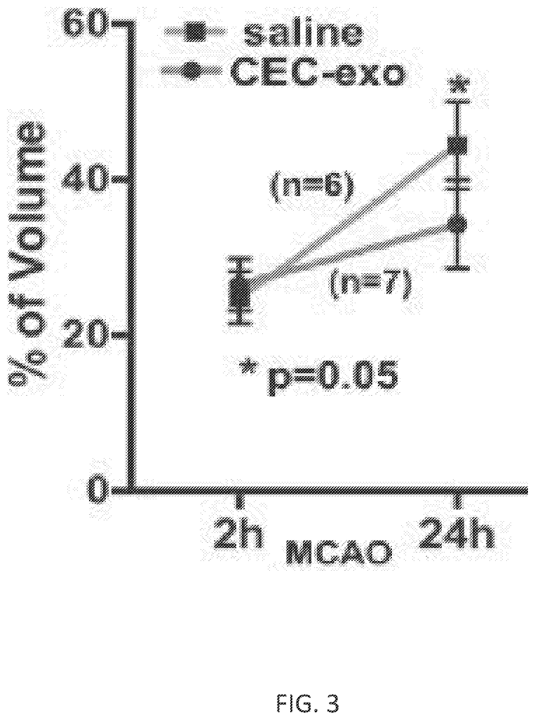

Early intravenous administration of CEC-exosomes attenuates ischemic lesion expansion. To examine whether early administration of CEC-exosomes limits ischemic lesion expansion, the inventors administered CEC-exosomes to ischemic rats via a tail vein at 1 h after embolic MCAO and then examined dynamic expansion of ischemic lesion 2 and 24 h after MCAO by means of MRI measurements, including apparent diffusion coefficient (ADC) and transverse relaxation time (T2) weighted images, respectively. CEC-exos reduced ischemic lesion volume by 30% measured by T2 weighted images 24 h after eMCAO compared to the saline treatment, although the lesion volume measured by DWI was comparable between saline and CEC-exos groups 2 h after eMCAO (FIG. 3). These data suggest that the early administration of CEC-exos (IV) attenuates ischemic lesion development.

The effect of age on tPA treatment. The inventors have demonstrated that administration of a full dose of tPA (10 mg/kg) to aged rats 2 h after embolic stroke dramatically increased the mortality rate to 67%, which precludes the use of full dose tPA in aged rats. The inventors have also demonstrated that administration of a reduced dose of tPA (5 mg/kg) to aged ischemic rats did not increase the mortality, but failed to reduce ischemic brain damage, and aggravated the neurovascular damage characterized by acute activation of vascular prothrombotic/proinflammatory signals. The effect of a low-dose (0.6 mg/kg) vs a standard-dose (0.9 mg/kg) alteplase on patients with acute ischemic stroke has been examined in a recent clinical trial, Enhanced Control of Hypertension and Thrombolysis Stroke Study (ENCHANTED, NCT01422616). The data from the ENCHANTED study showed that the low-dose alteplase achieves comparable results as the standard-dose. Thus, the inventors propose to employ tPA at a dose of 5 mg/kg for examining the effect of combination tPA with CEC-exosomes in aged rats starting at 2 h after embolic MCAO.

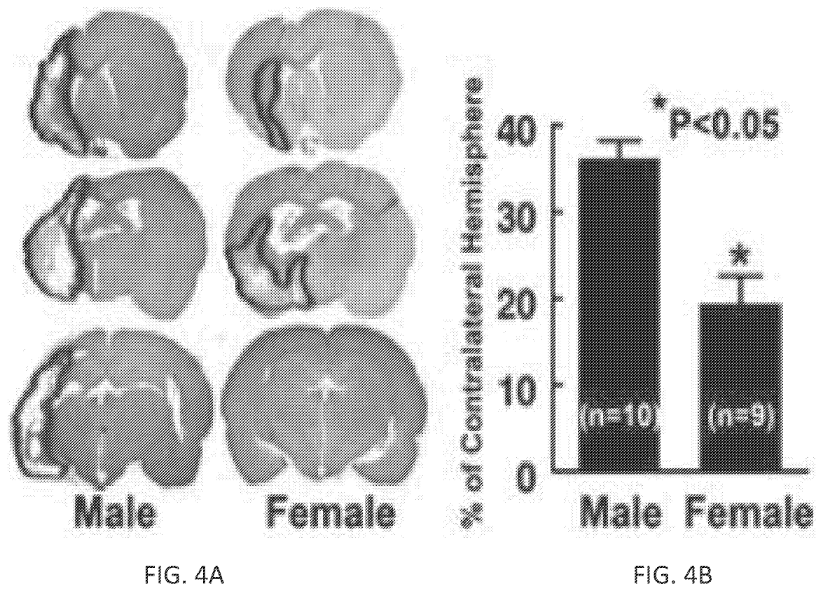

The effect of gender on embolic MCAO and tPA treatment. The data shown herein demonstrated that young adult female rats subject to embolic MCAO exhibited a smaller lesion volume compared to their male counterparts (See FIG. 4), which is in agreement with previous reports showing that ischemic lesion progression differs substantially between genders. Previous studies show advanced age in the female rats results in larger infarct size, increased mortality rate, and exacerbated BBB disruption, which are in agreement with the epidemiological observation in stroke that women account for the majority of stroke deaths. This age effect on female rats is likely related to termination of the estrous cycle, which occurs approximately at the age of 18 months. Thus, it is important to investigate the effect of CEC-exosomes and tPA on both male and female aged rats.

The goals of these experiments were to determine whether: 1) CEC-exosomes in combination with tPA (IV) administered after embolic MCAO or CEC-exosomes injected into the carotid artery (IA) immediately after transient MCAO, reduce ischemic neurovascular damage and improve neurological outcome, and 2) early (30 min after MCAO) administration (IV) of CEC-exosomes attenuates expansion of the ischemic core, leading to extension of the therapeutic window of adjuvant treatment of CEC-exosomes and tPA or IA CEC-exosomes to 6 hours or longer after MCAO (in animal in vivo models).

To examine the effect of combination treatment of CEC-exosomes/tPA on acute ischemic stroke in aged rats: The exosomes will be isolated from the supernatant of cultured cerebral endothelial cells (CECs) harvested from healthy young adult male rats (3-4 months) by means of differential ultracentrifugation. Aged (18 months) male and female rats will be subjected to embolic MCAO. Thirty min after MCAO, the Longa five point scores will be performed to assess neurological severity prior to the treatment. CEC-exosomes and tPA treatments will be initiated via a tail vein 2 h after MCAO and a second dose of CEC-exosomes will be administered (IV) 24 hours after MCAO. tPA at a dose of 5 mg/kg will be injected (IV) at 10% bolus, followed by continuous infusion for 30 min. For each gender, rats will be randomly assigned to one of the following 8 groups based on a pre-generated randomization schema: 1-2) CEC-exosomes (at concentrations of 1×10¹¹ or 1×10¹² exosomes/injection), 3) tPA, 4-5) CEC-exosomes (1×10¹¹ or 1×10¹² exosomes/injection) with tPA, 6) heat-inactive CEC-exosomes with tPA, 7) tPA with the liposome mimic of exosome lipid contents which have the same lipid components as exosomes, or 8) saline. In addition, additional rats subjected to sham operation are employed.

An array of commonly known behavioral tests (modified neurological severity score mNSS95, foot-fault, adhesive removal test) are performed to measure sensorimotor deficits weekly starting Id after MCAO. All animals are sacrificed 4 weeks after MCAO and their brains are cut into seven coronal sections. Infarct volume and hemorrhagic areas are measured on 7 H&E stained coronal sections under a light microscope according to published protocols. Infarct volume are measured according to Swanson's method that considers alteration of brain structure after ischemia including brain edema and atrophy and data of infarct volume are presented as percentage of the contralateral hemisphere. The inventors also document the incidence of death prior to sacrifice and the gross hemorrhage during the experiment period to evaluate the safety of the combination treatment. All measurements are performed blindly. A combination treatment is effective if there is any one or more of: (1) a significant reduction of infarct volume, (2) improvement of functional outcome, and (3) no evidence of augmentations of hemorrhage and mortality rate, compared to the controls (monotherapy of saline or tPA).

Without wishing to be bound by theory, the results are expected to show that the combination treatment with CEC-exosomes/tPA, but not tPA with heat-inactive CEC-exosomes or tPA with the liposome mimics, reduces infarct volume and improves neurological outcome without increasing cerebral hemorrhage. To examine the effect of CEC-exosomal lipids on acute stroke, the inventors will employ liposome mimics. The inventors have generated liposome mimics with the primary content of the exosome lipid/phospholipids using the thin-film hydration technique known in the art. The inventors' preliminary data showed that treatment with CEC-exosomes at 1×10¹¹ exosome particles/injection is effective to enhance tPA-induced thrombolysis.

The inventors thus include another high dose (1×10² exosomes/injection) of CEC-exosomes. In addition, one or more additional experiments will be added to measure the effect of additional doses of CEC-exosomes at 48 h and 72 h, because the secondary BBB leakage has been reported during 48 h and 72 h after stroke. Gender differences in coagulation and fibrinolytic factors have been reported in acute ischemic stroke. In animal models, female hormones have been shown to reduce stroke induced vascular damage. Thus, the inventors anticipate that compared to aged male rats, the combination treatment of CEC-exosomes and tPA will be at least equally effective in aged female rats. To mimic clinical practice in which patients are followed up from 90 days to 1 year after tPA or endovascular therapy, the inventors propose to sacrifice rats 28 days after MCAO. However, the inventors are aware that the majority of hemorrhages may not be detectable 4 weeks after MCAO due to infarct cavitation and phagocytosis of red blood cells by macrophages. In the subsequent experiments demonstrated in the present disclosure, the inventors may assay the effect of CEC-exosomes and tPA treatment on recanalization, reperfusion, hemorrhage, infarct volume, and neurological outcomes at 1 and/or 7 days after MCAO. The inventors do not expect sham-operated rats will exhibit stroke and neurological deficits. If this is the case, the inventors will not include sham-operated rats in the following experiments.

Examination of the therapeutic effect of CEC-exosomes injected into carotid artery (IA) immediately after transient MCAO on neurovascular damage and neurological outcome: To mimic thrombectomy, a model of transient MCAO with a filament is employed. Aged male and female rats are subjected to 2 h transient MCAO. Thirty min after MCAO, the Longa scores are performed to assess neurological severity prior to the treatment. Immediately following the filament withdrawal at 2 h after MCAO, CEC-exosomes at a dose determined previously, is administered via a catheter within the internal carotid artery and a second dose of CEC-exosomes is given via a tail vein at 24 h after MCAO. For each gender, rats are assigned to the following groups according to a pre-generated randomization schema: 1) CEC-exosomes, 2) heat-inactive CEC-exosomes, 3) the liposome mimics, or 4) saline. An array of behavioral tests are performed to measure sensorimotor deficits weekly starting Id after MCAO, as listed above. All animals are sacrificed 4 weeks after MCAO and histopathological analysis of infarct volume and hemorrhagic areas is performed as listed above.

Anticipated results, caveats, and alternative approaches: Without wishing to be bound by theory, the inventors expect that intra-arterial (IA) administration of CEC-exosomes results in substantial enhancement of reperfusion of the ischemic lesion, reduction of BBB leakage and infarct volume and improvement of neurological outcome, but does not increase cerebral hemorrhage. If the dose of CEC-exosomes based on IV administration is not appropriate for IA administration, the inventors will perform IA dose-response experiments. Alternatively, the inventors will administer additional CEC-exosomes (IV) 48 h and 72 h after MCAO. The inventors will utilize a device to retrieve a clot in the embolic MCAO model to mimic thrombectomy-induced recanalization.

To examine whether early administration (30 min after MCAO) of CEC-exosomes attenuates infarct expansion and consequently extends the therapeutic time window of combination of CEC-exosomes/tPA or IA CEC-exosomes. For the CEC-exosomes/tPA study, aged rats subjected to embolic MCAO are treated with the CEC-exosomes at a dose determined in the experimental procedures above, 30 min after MCAO via a tail vein and then treated (IV) with CEC-exosomes/tPA (5 mg/kg) 2 h, 4 h, and 6 h after MCAO. For each gender, rats are randomly assigned to one of 4 following groups: 1) CEC-exosomes early+CEC-exosomes/tPA, 2) heat-inactive CEC-exosomes early+CEC-exosomes/tPA, 3) CEC-exosomes/tPA, or 4) saline. For the IA CEC-exosomes study, aged rats subjected to transient MCAO are treated with the CEC-exosomes via a tail vein 30 min after MCAO and then treated (IA) with CEC-exosomes via an internal carotid artery following withdrawal of the filament 2 h, 4 h, and 6 h after MCAO. For each gender, rats are assigned to the following groups according to a pre-generated randomization schema: 1) CEC-exosomes early (IV)+IA CEC-exosomes, 2) heat-inactive CEC-exosomes early (IV)+IA CEC-exosomes, 3) IA CEC-exosomes, or 4) saline.

Behavioral tests are performed at 1 and 7 days after MCAO. All animals will be sacrificed 7 days after MCAO. The primary endpoints will be infarct volume, hemorrhagic transformation, neurological outcome, and mortality rate, which will be measured as outlined in experimental sections above.

Anticipated results, caveats, and alternative approaches: Based on the inventor's preliminary data, the inventors expect that IV administration of CEC-exosomes 30 min after embolic MCAO will reduce ischemic core expansion, which leads to enhancement of the therapeutic effect for the combination of CEC-exosomes/tPA given 2 h after MCAO in the aged rats animal model and to extend the therapeutic window of tPA treatment to 4 h or 6 h compared to a single tPA agent treatment alone. In contrast, early administration of the heat-inactive CEC-exosomes will abolish the enhanced therapeutic effect of the combination of CEC-exosomes/tPA. If the inventors fail to detect any therapeutic effect and/or observe increased hemorrhagic complication and mortality rate at the 4 h therapeutic window, the inventors will not proceed to investigate the 6 h window.

For the IA CEC-exo study, the inventors expect that IV administration of CEC-exosomes 30 min after onset of MCAO will reduce infarct expansion, and will thereby supplement and enhance the therapeutic effect for IA CEC-exosomes given 2 h and 4 h after transient MCAO compared to IA CEC-exosomes alone. Moreover, early IV administration of CEC-exosomes will extend efficacious treatment times of IA CEC-exosomes to 6 h post stroke compared to IA CEC-exosomes alone. If data of early IV at 30 min and IA at 6 h are positive, the inventors will extend IA CEC-exosomes to 8 h or longer duration of transient MCAO. The inventors have demonstrated in embolic and filament models that infarction is mature 7 days after MCAO and that the therapeutic effect on neurological outcomes can be detected during 7 days of MCAO effect in this model. The inventors thus sacrifice rats 7 days after MCAO.

### Example 3. CEC Exosomal Cargo miRNAs Contribute to CEC-Exosomes-Amplified Thrombolysis Leading to Blocking and Prevention of Neurovascular Damage

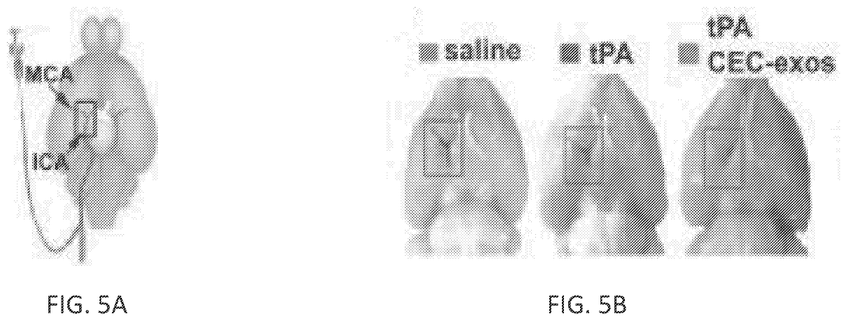

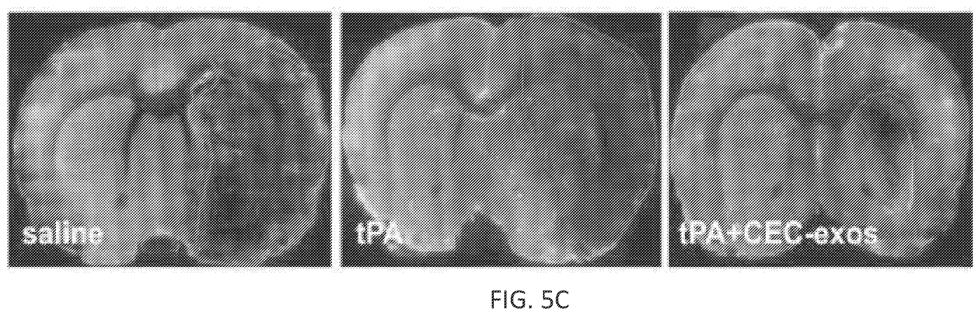

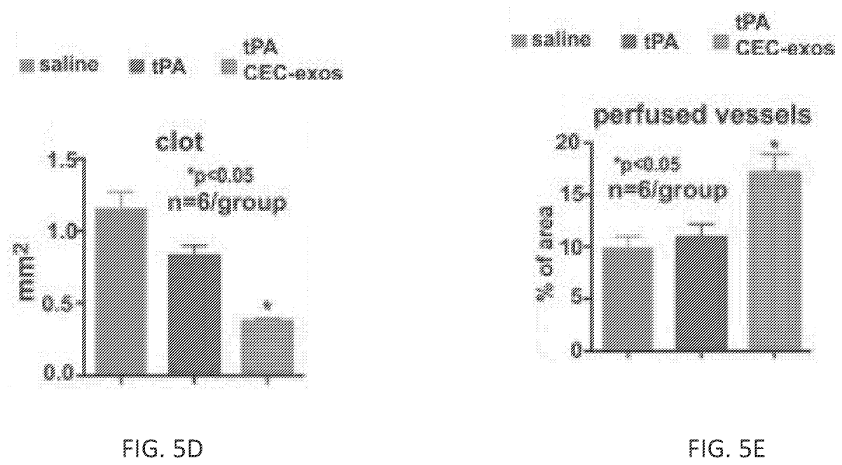

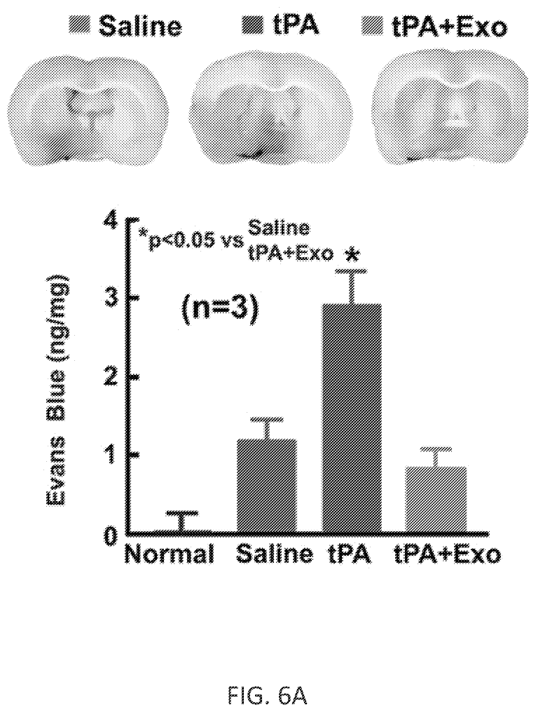

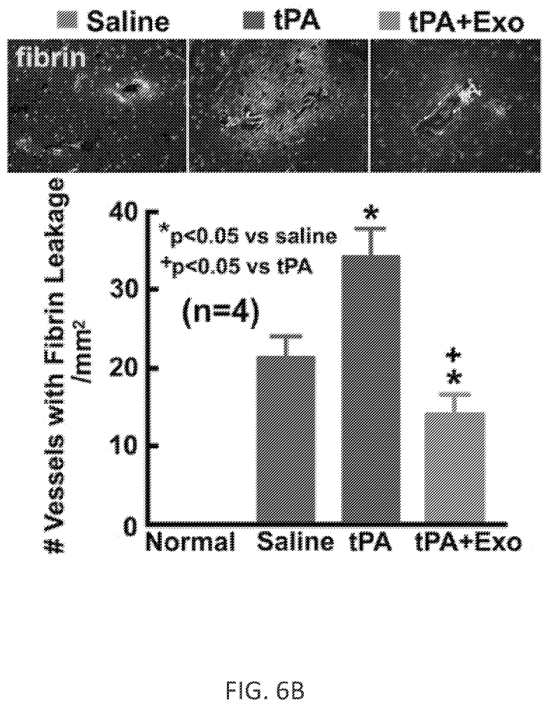

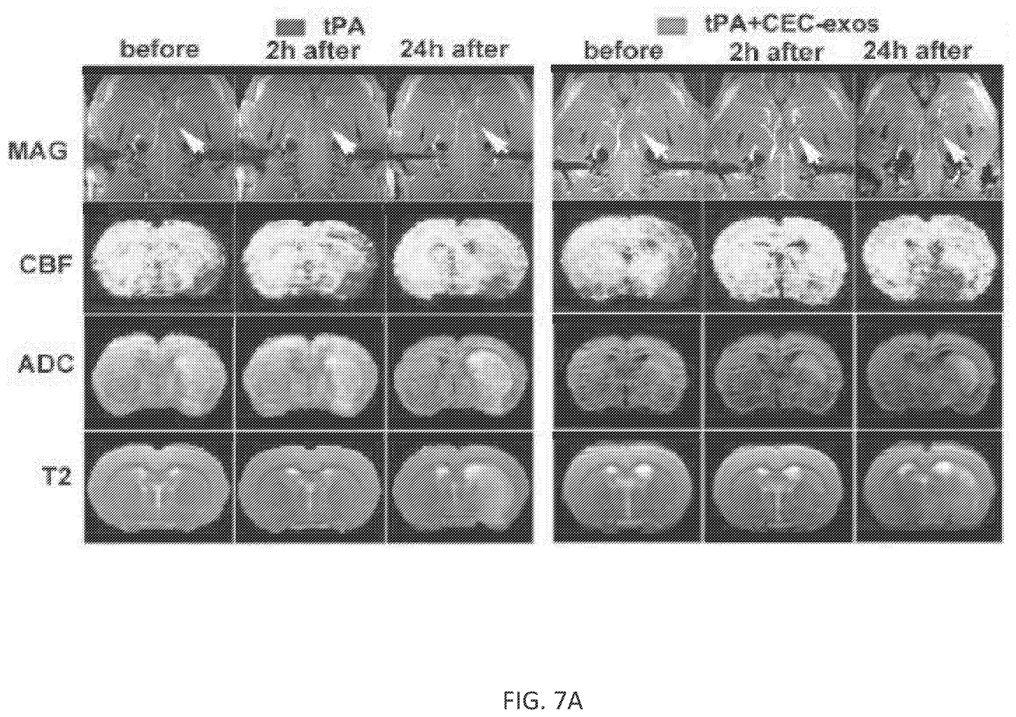

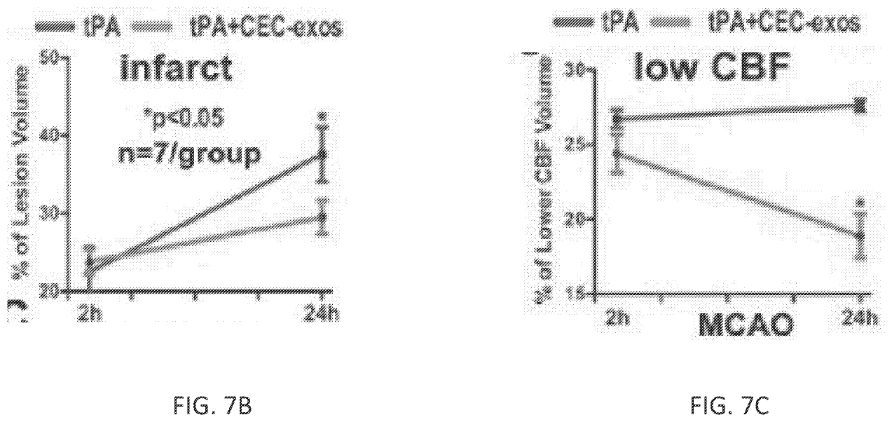

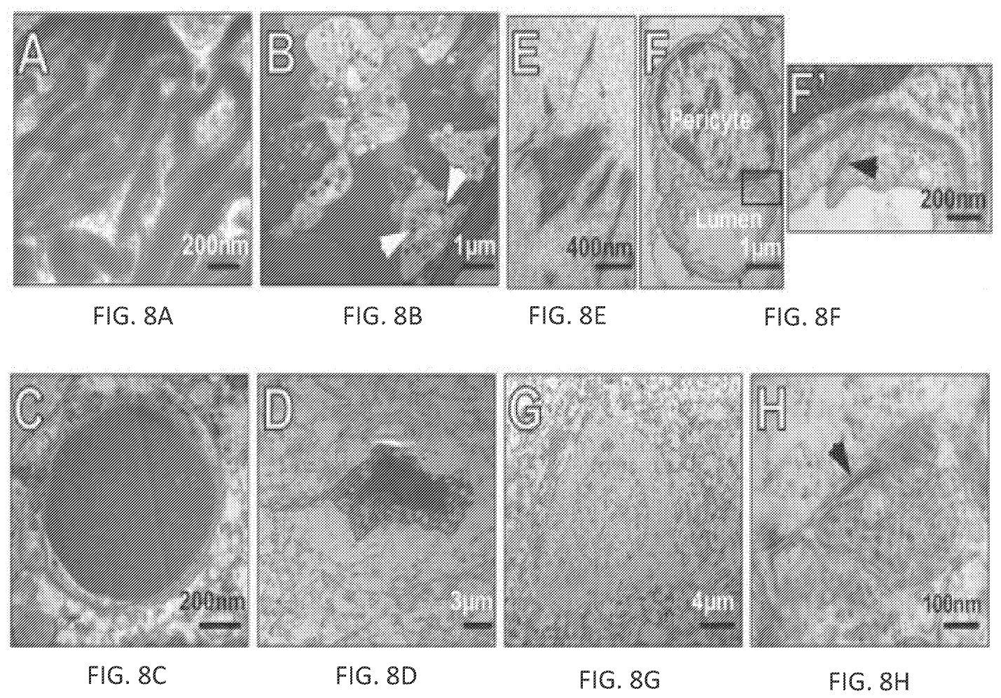

CEC-exosomes in combination with tPA promote recanalization, enhance microvascular patency and integrity, and reduce ischemic brain damage. To examine the effect of CEC-exosomes on recanalization of the occluded MCA and its downstream microvascular perfusion, another set of experiments were performed in which rats were sacrificed 24 h after MCAO. Analysis of embolus size at the origin of the occluded MCA revealed that the CEC-exosomes in combination with tPA significantly reduced the embolus size compared to monotherapy of tPA (See FIGS. 5B, and 5D). To examine the patency of downstream microvessels, the inventors injected (IV) FITC-dextran to the rats and sacrificed the rats 5 min after the injection. The inventors have demonstrated that FITC-dextran within plasma perfuses all patent cerebral vessels. 3-D laser scanning confocal microscopy (LSCM) analysis showed that the combination treatment significantly increased FITC-dextran perfused microvessels fed by the MCA (See FIGS. 5C, and 5E). These data indicate that CEC-exos augment tPA clot thrombolysis and brain tissue perfusion. To examine the effect of CEC-exos on BBB leakage, Evans blue was intravenously administered at 24 h after eMCAO. Evans blue binds to albumen. Quantitative measurements (FIG. 6A) showed that tPA monotherapy significantly increased brain Evans blue levels compared to the saline treatment. However, CEC-exos in combination with tPA robustly reduced brain Evans blue (FIG. 6A) compared to monotherapy of tPA. Double immunofluorescent staining showed that CEC-exos in combination with tPA also significantly reduced extravascular fibrin deposition compared to the saline and tPA alone (FIG. 6B). These data indicate that CEC-exos in combination with tPA reduce BBB leakage. Moreover, to longitudinally and non-invasively measure the therapeutic effect of CEC-exos in combination with tPA, MRI measurements were performed in the same rat before and after the treatment. MRI data demonstrated that compared to tPA monotherapy, treatment with CEC-exos in combination with tPA significantly promoted recanalization of the occluded MCA measured with magnetic resonance angiography (MRA), increased downstream perfusion assayed by reduction of low CBF area, and decreased infarct volumes measured with apparent diffusion coefficient (ADC), and transverse relaxation time (T2) 24 h after eMCAO (FIG. 7). Ultrastructural analysis with transmission electron microscopy (TEM) showed that the combination treatment led to few fibrin bundles with inactivated platelets in emboli of the occluded MCA, open lumen of downstream capillaries with intact tight junction, and intact neurons with synaptic complex within ischemic lesion, whereas rats treated with tPA alone exhibited dense fibrin bundles and fibrin-adhered active platelets in emboli of the occluded MCA, capillaries occluded by red blood cells and damaged endothelial cells and dead neurons in ischemic lesion (see FIG. 8). Collectively, these data strongly suggest that CEC-exosomes facilitate tPA-induced thrombolysis of the occluded MCA and downstream microvascular reperfusion, leading to reduction of ischemic neuronal death and consequently to improved neurological function.

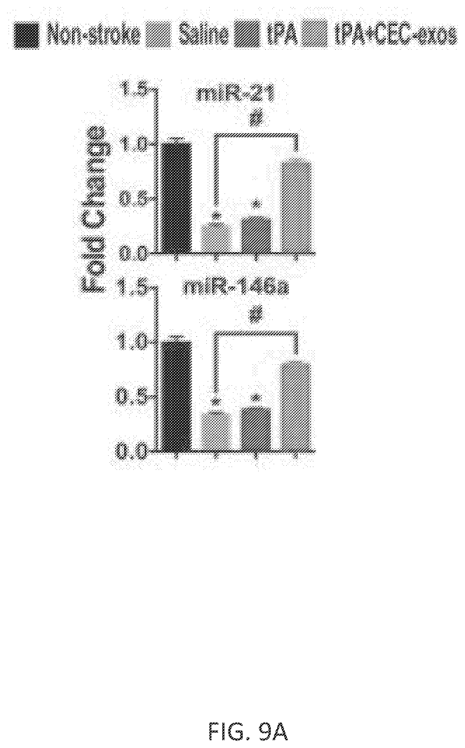

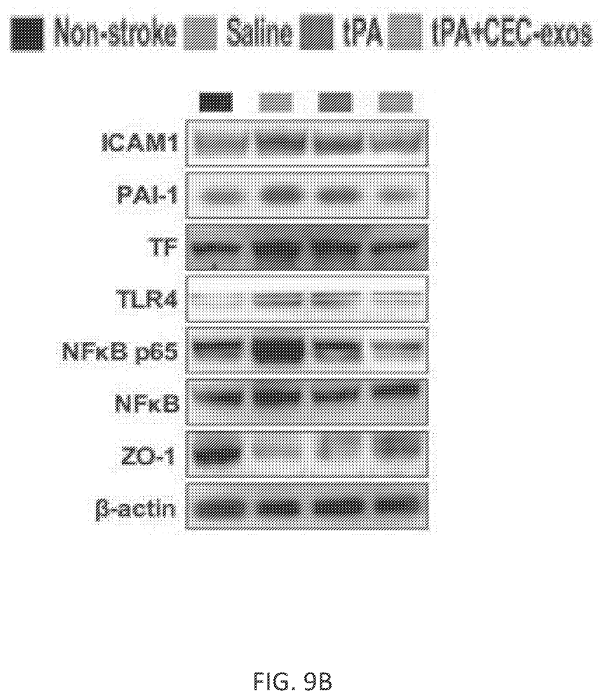

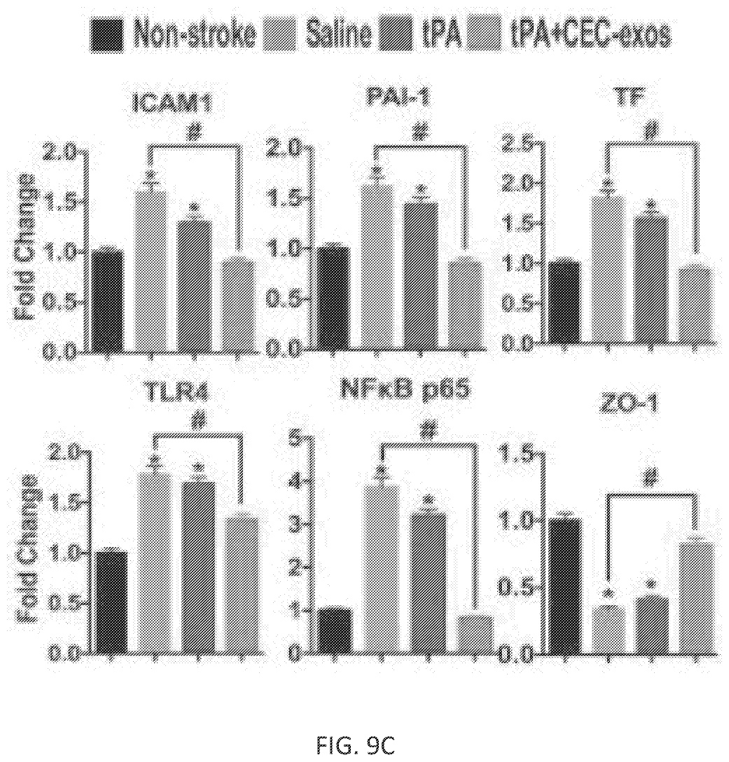

CEC-exosomes elevate miR-21 and miR-146a and reduce proteins that promote thrombosis and vascular dysfunction in cerebral endothelial cells. To examine the effect of CEC-exosomes in combination with tPA on miRs, miRs were analyzed in primary cerebral endothelial cells isolated from the ischemic blood vessels of adult rats subjected to 24 h eMCAO (n=3 rats/group) using quantitative RT-PCR (qRT-PCR). The inventors found that treatment with saline or tPA significantly reduced miR-19a, -21 and -146a compared to non-ischemic rats, whereas CEC-exos in combination with tPA reversed levels of the three miRs close to non-ischemic rats (FIG. 9A). Western blot analysis of the endothelial cells from the same set of rats showed that compared to non-ischemia, saline or tPA treatment increased levels of toll-like receptor 4 (TLR4), intercellular adhesion molecule-1 (ICAM-1), plasminogen activator inhibitor 1 (PAI-1), tissue factor (TF), and phosphorylated NF-κB (pNF-κB), but reduced tight junction protein zonula occludens 1 (ZO1), which were reversed by the combination treatment (FIG. 9 B, C). These data indicate that CEC-exos act directly on endothelial cells and the entry of exosomes within the endothelial cells directly affect the molecular content of endothelial cells and the structure of the microvasculature.

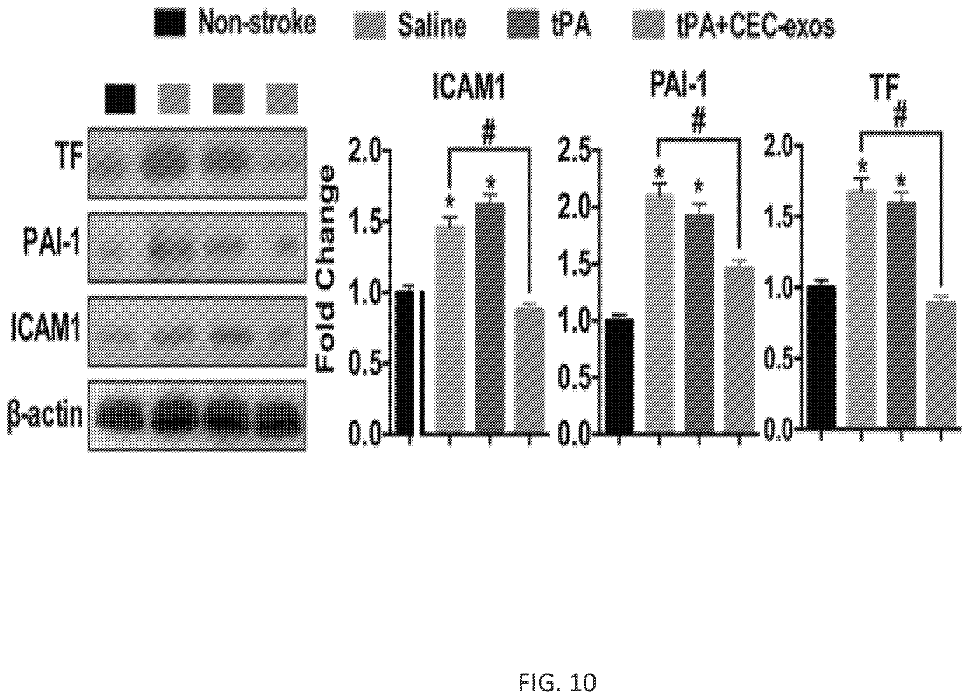

CEC-exos reduce pro-thrombotic proteins in plasma. In addition to cerebral endothelial cells, the inventors also found that stroke and tPA significantly increased plasma levels of TF, PAI-1, and ICAM-1, whereas CEC-exos in combination with tPA significantly reduced these three protein levels (FIG. 10). Increased circulating TF is linked to an unfavorable outcome in patients with acute stroke. Patients with acute stroke show increased plasma levels of PAI-1. Activated endothelial cells and macrophages express TF. Thus, CEC-exos in combination with tPA directly promote intravascular conditions which enhance tissue perfusion.

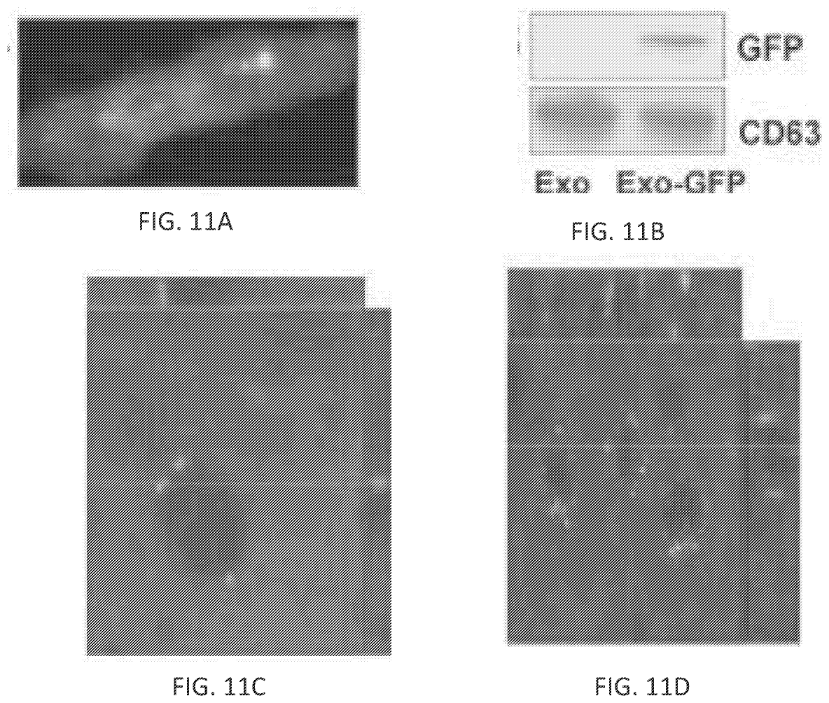

CEC-exosomes intravenously administered are localized to endothelial cells of cerebral blood vessels and cross the BBB. To examine whether intravenously administered CEC-exos interact with and enter cerebral endothelial cells and brain parenchymal cells, the inventors generated CEC-exos carrying CD63-GFP (CEE/CD63-GFP), from the supernatant of cerebral endothelial cells transfected with the CD63-GFP plasmid (FIG. 11 A, B). CD63 is enriched in the exosome membrane and has been used as an exosomal marker. CEC-exos/CD63-GFP were administered to the rat via a tail vein, and sacrificed 4 h after the injection. 3D confocal microscopic analysis showed puncta GFP signals within cerebral endothelial cells (FIG. 11C) and neurons (FIG. 11D). These data indicate that CEC-exos are internalized by cerebral endothelial cells, cross the BBB and enter parenchymal cells.

Without wishing to be bound by any particular theory, it is believed that most of the therapeutic effects of exosomes are attributed to their cargo miRNAs. Various embodiments of the present examples are presented herein to investigate whether CEC-exosome cargo miRNAs underlie the therapeutic effect of CEC-exosomes on enhancement of thrombolysis and reduction of neurovascular damage.

To examine whether CEC exosomal cargo miRNAs contribute to the therapeutic effect of CEC-exosomes, Dicer knockdown exosomes (CEC-exosomes/Dicer) are generated. Briefly, cerebral endothelial cells harvested from young adult male rats are transfected with siRNAs against Dicer1 (Dharmacon) or with a scrambled siRNA by means of the electroporation. Exosomes are isolated from these transfected endothelial cells, named as CEC-exosomes/Dicer or CEC-exosomes/Scr, respectively. CEC-exosomes/Scr and are used as control. Dicer is essential for miRNA biogenesis and ablation of Dicer in parent cells leads to 88% reduction of individual miRNAs within exosomes. miRNA microarrays are performed and qRT-PCR is used to analyze the profile and levels of miRNAs, respectively, in CEC-exosomes/Dicer and CEC-exosomes/Scr. After verification of reduction of exosomal miRNAs in particular for miRNAs involved in the network detected in preliminary data, aged male and female rats are subjected to embolic MCAO. Neurological severity prior to the treatment is measured. CEC-exosomes at a dose determined in in experiments described above and tPA treatments are initiated via a tail vein 2 h after MCAO and a second dose of CEC-exosomes is administered (IV) 24 h after MCAO. For each gender, rats are to be assigned to the following groups according to a pre-generated randomization schema: 1) CEC-exosomes/Dicer/tPA, 2) CEC-exosomes/Scr/tPA, 3) naive CEC-exosomes/tPA, or 4) saline. An array of behavioral tests will be performed to measure sensorimotor deficits 1 d and 7 d after MCAO, as shown in the above examples. All animals are sacrificed 1 week after MCAO and histopathological analysis of infarct volume and hemorrhagic areas is performed according to published protocols (See for example, Zhang Z, Zhang L, Yepes M, Jiang Q, Li Q, Arniego P, et al. Adjuvant treatment with neuroserpin increases the therapeutic window for tissue-type plasminogen activator administration in a rat model of embolic stroke. Circulation. 2002; 106(6):740-5, and Jiang Q, Zhang R L, Zhang Z G, Knight R A, Ewing J R, Ding G, et al. Magnetic resonance imaging characterization of hemorrhagic transformation of embolic stroke in the rat. J Cereb Blood Flow Metab. 2002; 22(5):559-68, (the disclosures of which are incorporated herein by reference in their entireties).

It is anticipated that Dicer-siRNA will substantially reduce the majority of CEC exosomal miRNAs as the inventors previously demonstrated that ablation of Dicer in adult neural stem cells resulted in remarkable decrease their exosomal cargo miRNAs. The inventors also expect that administration of CEC-exosomes/Dicer will substantially attenuate observed therapeutic effect of CEC-exosomes compared to control scrambled siRNA or naïve exosomes. These data will provide insight into the cause-effect of CEC exosomal miRNAs. Ago2 is attenuated if substantial reduction of exosomal miRNAs is not achieved by knocking down of Dicer. Ago2 is one of the main components of RISC and plays a key role in mediating the activity of miRNA-guided mRNA cleavage or translational inhibition. The inventors have previously demonstrated that Ago2 knockdown exosomes abolish exosomal miRNA mediated axonal growth of cortical neurons. The inventors have taken into consideration that exosomes contain proteins and do not exclude the possibility that exosomal proteins may play a role in the therapeutic effect of CEC-exosomes.

In another experimental example, CEC exosomal cargo miRNAs are examined to determine whether they contribute to rapid recanalization and microvascular perfusion. In another set of experiments, ischemic male rats are treated as provided in the experiments described above. MRI is used to dynamically measure recanalization of the occluded MCA, and vascular patency and integrity in male aged rats. Briefly, aged male rats are subjected to embolic MCAO by placing an Evans blue pre-labeled embolus that emits red fluorochrome, as previously described. Rats are assigned to the following groups according to a pre-generated randomization schema: 1) CEC-exosomes/Dicer/tPA, 2) CEC-exosomes/Scr/tPA, 3) naive CEC-exosomes/tPA, or 4) saline. Recanalization of the occluded MCA, CBF and vessel leakage, brain edema, and ischemic lesion are examined with well-established MRI indices. MRI measurements include MRA for recanalization, perfusion weighted imaging for CBF, blood-to-brain transfer constant of GD-DTPA (Ki) for BBB leakage, ADC, and transverse relaxation time (T2) for ischemic lesion volume. MRI measurements are performed before the treatment, and after the treatment at 1 h and 24 h. Immediately after the last MRI measurement, rats are administered FITC-dextran (IV) 5 minutes before sacrifice. FITC-perfused vessels will be analyzed with LSCM as indicted in the experimental examples provided above. Vascular leakage of albumin and fibrin on adjacent immunostained coronal sections will be measured as outlined in in the experimental examples provided above. Thus, these MRI and histopathological data provide strong data regarding the effect of reduced cargo miRNAs of CEC-exosomes on reperfusion, BBB leakage and development of infarction.

At histopathological levels, recanalization and cerebral microvascular perfusion and integrity is further examined in these rats. Immediately after the last MRI measurement, rats are administered FITC-dextran (IV) 5 minutes before sacrifice. After sacrifice, their brains are cut into a series of coronal sections (100-pm/section). Four coronal sections (1.0 to −1.0 mm from bregma) per rat are used for the measurement of residual embolus and FITC-dextran perfused vessels by means of LSCM according to the inventors published protocols. By combining results of the MR angiogram and of confocal images for the Evans blue (red) and FITC-dextran (green) at the origin of the MCA, data is acquired regarding thrombolysis and recanalization of the occluded MCA. By analysis of data of MRI CBF and FITC-dextran perfused vessels within the MCA territory, brain reperfusion information is acquired for further analysis. Additionally, double immunofluorescent staining is performed on adjacent coronal sections according to published protocols (the disclosures of which are incorporated herein by reference in their entireties). For vascular thrombosis, the number endothelial barrier antigen (EBA) positive cerebral vessels with intravascular deposition of fibrin (EBA+/fibrin+), platelets (EBA+/thrombocytes+), and leukocytes (EBA+/myeloperoxidase, MPO+) is measured and analyzed. For vascular leakage, the number of cerebral vessels (EBA+vessels) with extravascular albumin (albumin+) and fibrin (fibrin+) deposition and cerebral vessels with basal lamina collagen IV (EBA+/collagen IV+) is measured and analyzed. By correlating these immunochemistry data with GD-DTPA contrast results, information of BBB disruption is obtained. Brain hemorrhage is also measured on adjacent sections. Collectively, these MRI and histopathological data provides strong data to determine whether CEC exosomal cargo miRNAs contribute to the therapeutic effect of CEC-exosomes in combination with tPA on recanalization, reperfusion, BBB leakage and development of infarction.

Without wishing to be bound by theory, it is expected that the combination of naïve CEC-exosomes/tPA, when administered intravenously, promotes recanalization of the occluded MCA and enhance downstream microvascular perfusion, and reduce BBB leakage and ischemic lesion volume without increasing brain hemorrhage. In contrast, these therapeutic effects of naïve CEC-exosomes/tPA are significantly attenuated in ischemic rats treated with CEC-exosomes/Dicer/tPA, which provides strong support of the role of CEC exosomal cargo miRNAs in mediating the therapeutic effect of CEC-exosomes on recanalization, reperfusion, and infarction. Endogenous thrombolysis occurs 24 h after MCAO, which results in clot lysis. To accurately analyze the combination therapy on recanalization, rats are sacrificed 24 h after MCAO. If the inventors fail to detect that the treatment with CEC-exosomes/Dicer/tPA affects CEC-exosomes/tPA-enhanced rapid recanalization and reperfusion, but abolishes the effect of CEC-exosomes/tPA on reduction of ischemic lesion, this would imply that CEC exosomal cargo miRNAs primarily act on protecting ischemic brain cell damage as CEC-exosomes cross the BBB and are presented in neurons as demonstrated in preliminary data (shown in FIG. 11D). Subsequently, detailed histological analysis is performed to examine ischemic neuronal damage. Alternatively, the effect of CEC-exosomes/tPA and CEC-exosomes/Dicer/tPA on stroke-increased angiogenesis and neurogenesis is examined. The inventors have previously demonstrated that exosomal cargo miRNAs affect angiogenesis and neurogenesis. In these experimental examples described herein, male aged rats are employed. However, if data from the above experimental results demonstrate that gender affects the therapeutic effect of CEC-exosomes in combination with tPA, the instant experiments with female aged rats are performed and compared to the male aged rats.

### Example 4. CEC-Exosomes Cargo miR-19a, miR-21 and miR-146a Contribute to the Therapeutic Effect of CEC-Exosomes on Stroke-Induced Neurovascular Damage by Suppressing a Network of Pro-Vascular Injury and Thrombogenicity Genes, Including Cell Adhesion, Tissue Factor and Toll-Like Receptor Signaling

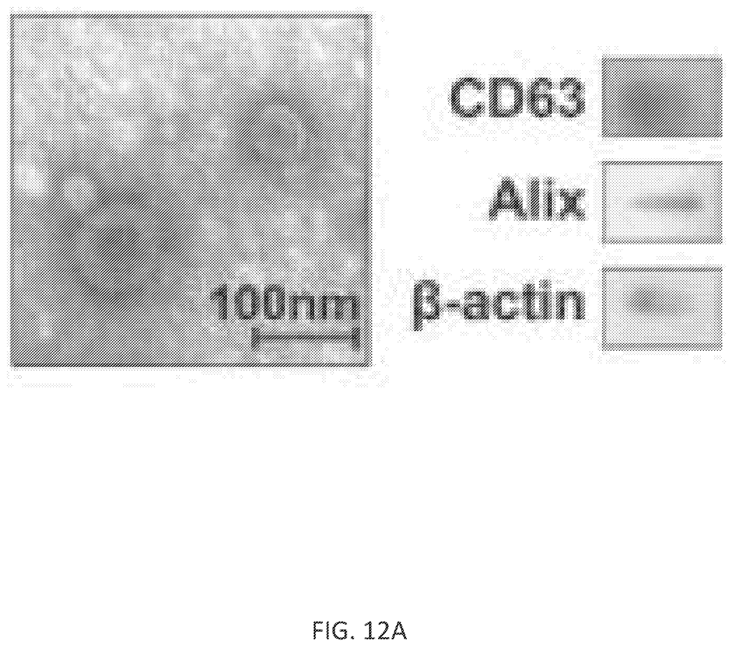

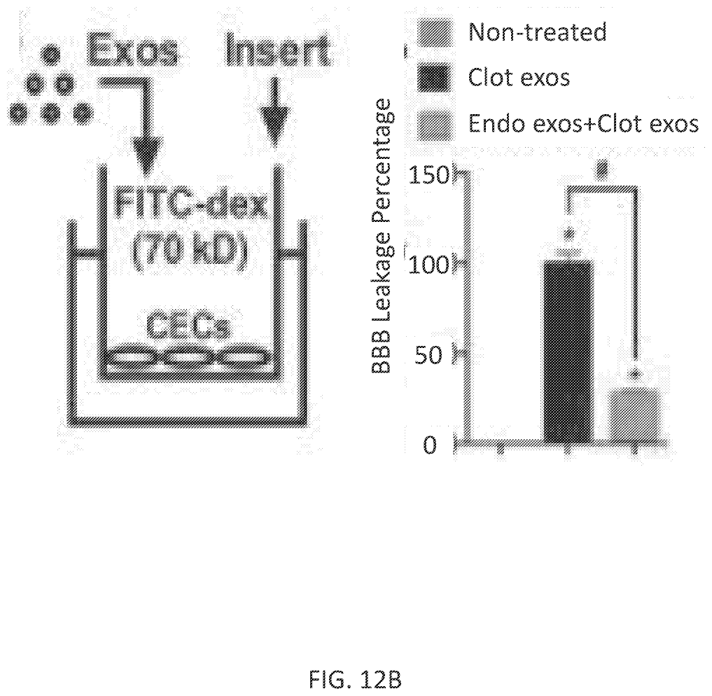

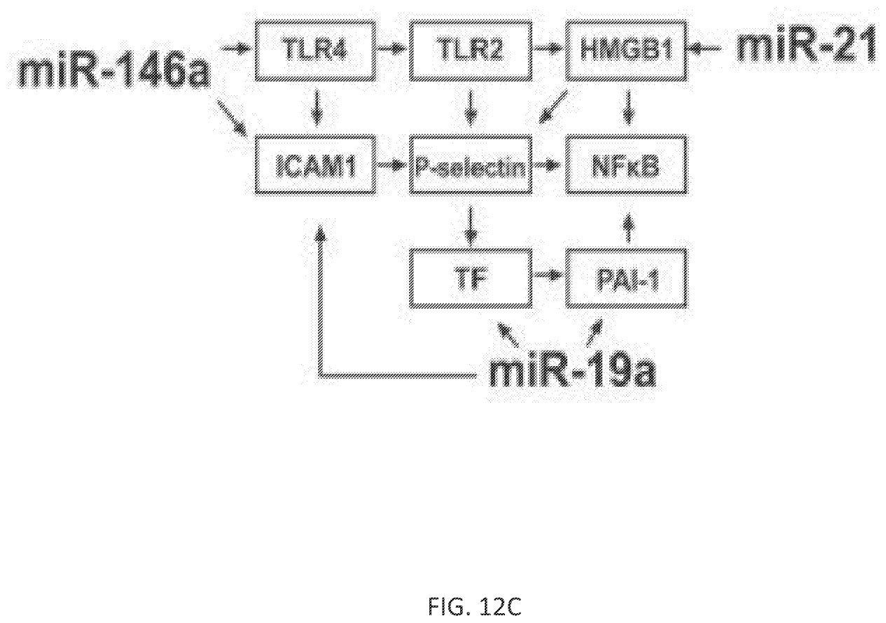

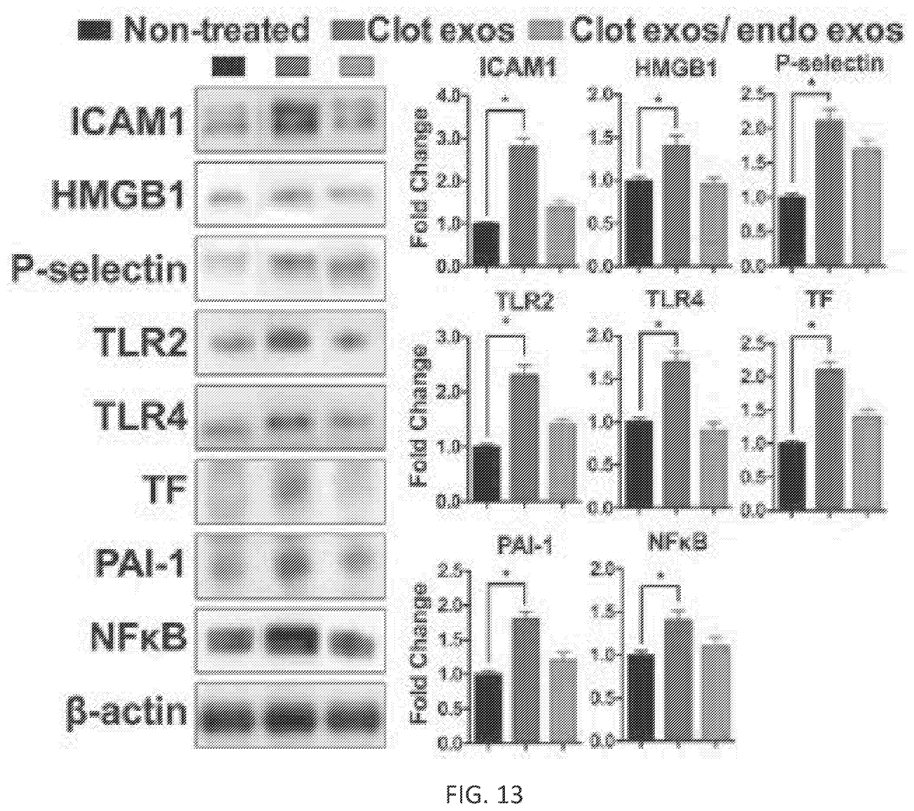

Stroke patient clot-injured exosomes induce healthy cerebral endothelial cells to activate a network miRNAs/proteins that promote vascular injury and thrombogenicity. The inventors have isolated exosomes from thrombi retrieved by thrombectomy from patients with acute stroke. TEM revealed that these exosomes had doughnut morphology and mean diameter ˜100 nm (see FIG. 12A). Western blot analysis showed the presence of CD63 and Alix markers in these isolated CEC clot-injured exosomes (as shown in FIG. 12A). Thus, these are exosomes, which differ from microparticles released from the plasma membrane during budding or shedding and microparticles having diameters ranging between 100 nm and 1 μm. Incubation of healthy human cerebral endothelial cells (CEC) with stroke patient clot-injured exosomes increased BBB permeability measured by an in vitro BBB method showing an increase of FITC-dextran crossing the endothelial cell layer (see for example, the results shown in FIG. 12B). Stroke patient-derived exosomes induce healthy cerebral endothelial cells to activate a network of miRs/proteins that promote thrombogenicity and BBB leakage. Quantitative RT-PCR analysis showed that the patient-derived exosomes triggered the endothelial cells to significantly (p<0.05) reduce miR-19a (0.4±0.01 vs 1), -21 (0.5±0.06 vs 1), and -146a (0.4±0.03 vs 1) and to increase proteins of TLR2/4 and HMGB1 as well as their related proteins of ICAM1, P-selectin, PAI-1, TF, and NF-κB (FIG. 13). However, CEC-exos significantly reversed levels of proteins altered by patient-derived exosomes (FIG. 13). These data suggest that CEC-exos repress proteins in the network in cerebral endothelial cells to reduce BBB leakage and thrombosis, consequently leading to increase of cerebral perfusion and reduction of infarction. Bioinformatics analysis revealed that miR-19a, -21 and -146a form a network with either direct or indirect target genes coding proteins that are highly involved in pro-thrombosis including TF, PAI-1, ICAM-1, and TLR/NF-κB signaling. These molecular data are parallel to the preliminary findings of functional and histological data, suggesting that CEC-exos transfer their cargo miRNAs, in particular miR-19, -21 and -146a, to cerebral endothelial cells to suppress stroke- and tPA-triggered pro-thrombotic genes in endothelial cells, leading to improvement of vascular patency and BBB integrity. (see FIG. 12C). These data exemplified herein, demonstrate for the first time, that stroke patient clot-injured exosomes play an important role in mediating ischemic neurovascular damage.

Healthy CEC-exosomes inactivate the thrombogenicity network of miRNAs/proteins. Healthy CEC-exosomes significantly reverse miRNAs and proteins altered by stroke patient clot-injured exosomes and BBB leakage as demonstrated and illustrated in FIG. 12B. These in vitro patient data are consistent with rat preliminary data present in FIGS. 9 and 11.

Using a miRNA array, miRNAs within CEC-exosomes were analyzed and it was found that miRNAs were enriched in CEC-exosomes and that miR-19a, miR-21 and miR-146a were among the top 10 enriched miRNAs. Thus, the goal of the present example is to investigate whether CEC-exosomes deliver their cargo miR-19a, miR-21, and miR-146a to cerebral endothelial cells to repress the network of miRNAs/proteins.

The objective of the present experimental example was to examine whether CEC-exosomes with reduced miR-19a, miR-21, and miR-146a attenuate the inhibitory effect of naïve CEC-exosomes on proteins in the network, consequently leading to the abolition of naïve CEC-exosomes induced rapid recanalization and microvascular perfusion. To generate CEC-exosomes containing the reduced miRNAs, cerebral endothelial cells harvested from young adult male rats will be transfected with siRNAs against miR-19a, miR-21, and miR-146a (Dharmacon) by means of the electroporation. Endothelial cells transfected with scramble RNAs will be used as a control group. Exosomes will be isolated from supernatants of these cultured cerebral endothelial cells. Reduction of miR-19a, miR-21, and miR-146a within CEC-exosomes will be verified with qRT-PCR prior to in vivo administration. Aged male rats will be subjected to embolic MCAO.

Thirty min after MCAO, the Longa scores are performed to assess neurological severity prior to the treatment. CEC-exosomes at a dose determined in the above examples and tPA treatments are introduced via a tail vein 2 h after MCAO and a second dose of CEC-exosomes is administered (IV) 24 h after MCAO. Rats are assigned to the following groups according to a pre-generated randomization schema: 1) CEC-exosomes (containing miR-19a, miR-21, and miR-146a)/tPA, 2) CEC-exosomes/Scr/tPA, 3) naive CEC-exosomes/tPA, or 4) saline. Recanalization of the occluded MCA, CBF and vessel leakage, brain edema, and ischemic lesion is examined before the treatment, and after the treatment at 1 h and 24 h by means of MRI, as outlined in the examples provided above. Histological analysis is performed for FITC-dextran perfused vessels, vascular thrombosis, and BBB leakage, as outlined in the relevant example above.

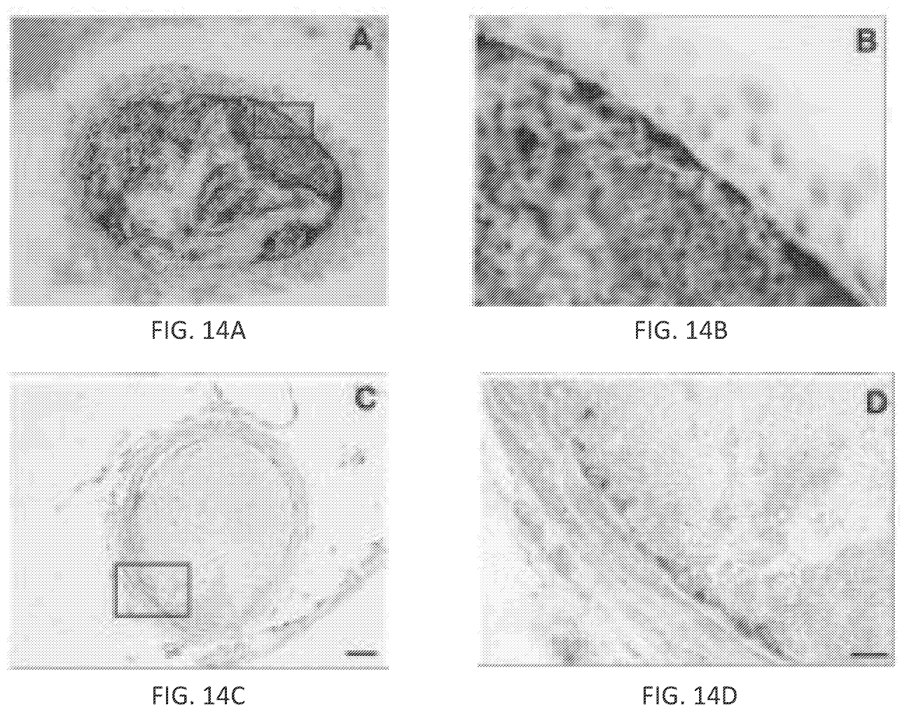

The effect of CEC-exosomes with reduced miR-19a, miR-21, and miR-146a on their target genes coding HMGB1, ICAM-1, P-selectin, PAI-1, TLR2/4 and TF proteins in cerebral endothelial cells are examined. Briefly, primary cerebral endothelial cells are isolated from cerebral microvessels of cohorts of rats sacrificed 24 h after MCAO as described previously 142. Using Taqman primers specific to mature miRNAs and mRNAs, levels of miR-21 and -146a, and mRNAs of HMGB1, ICAM-1, P-selectin, PAI-1, TLR2/4 and TF in cerebral endothelial cells are analyzed as described according to Zhang L, Chopp M, Liu X, Teng H, Tang T, Kassis H, et al. Combination therapy with VELCADE and tissue plasminogen activator is neuroprotective in aged rats after stroke and targets microRNA-146a and the toll-like receptor signaling pathway. Arterioscler Thromb Vasc Biol. 2012; 32(8):1856-64 and Liu X S, Chopp M, Pan W L, Wang X L, Fan B Y, Zhang Y, et al. MicroRNA-146a Promotes Oligodendrogenesis in Stroke. Mol Neurobiol. 2016, 10.1007/s12035-015-9655-7, the disclosures of which are incorporated herein by reference in their entireties. Endothelial protein levels of the noted mRNAs are measured by Western blot analysis. As the preliminary data suggests, the network of miRNAs/proteins affect formation of NETs, the effect of alteration of this network on NETs in emboli localized to the MCA is examined. Immunohistological staining is performed with antibodies against citrullinated histone H3, neutrophil elastase, and MPO on brain coronal sections containing emboli localized to the MCA (see for example, FIG. 14, as shown in the inventors' published article: Zhang Z G, Zhang L, Tsang W, Goussev A, Powers C, Ho K, et al. Dynamic platelet accumulation at the site of the occluded middle cerebral artery and in downstream microvessels is associated with loss of microvascular integrity after embolic middle cerebral artery occlusion. Brain Res. 2001; 912(2):181-94, (the disclosure of which is incorporated herein by reference in its entirety). These miRNA and protein data is correlated to recanalization and tissue reperfusion data generated with MRI and histopathological analysis.

Experiment 3a-2: To directly examine whether CEC-exosomes with reduced miR-19a, miR-21, and miR-146a attenuate the inhibitory effect of naïve CEC-exosomes on proteins in the network. The inventors will perform in vitro experiment. Briefly, CEC-exosomes (containing miR-19a, miR-21, and miR-146a) (0.5 ml of 3×10⁸ exosomes/mL) will be added into healthy human cerebral endothelial cells (density of 1×10⁴) treated with patient-clot-derived exosomes. The endothelial cells will be collected 24 h after CEC-exosomes (containing miR-19a, miR-21, and miR-146a) treatment and miRNA levels of miR-19a, miR-21, and miR-146a and the proteins noted in the network is analyzed. BBB leakage is also analyzed according to a published protocol as described in Niego B, Freeman R, Puschmann T B, Turnley A M, Medcalf R L. t-PA-specific modulation of a human blood-brain barrier model involves plasmin-mediated activation of the Rho kinase pathway in astrocytes. Blood. 2012; 119(20):4752-61, (the disclosure of which is incorporated herein by reference in its entirety), and as shown in FIG. 13. There will be four groups: 1) CEC-exosomes (miR-19a, miR-21, and miR-146a), 2) CEC-exosomes/Scr, 3) naive CEC-exosomes, or 4) PBS.

Based on the preliminary data shown herein, CEC-exosomes carrying reduced miR-19a, miR-21, and miR-146a will not suppress their listed target proteins and attenuate the therapeutic effect of naïve CEC-exosomes on enhancement of brain tissue perfusion. Without being limited to any particular theory, it is believed that naïve CEC-exosomes will significantly reduce formation of NETs, which will be substantially diminished by CEC-exosomes with reduced levels of these three miRNAs (miR-19a, miR-21, and miR-146a). The inventors also expect that in vitro CEC-exosomes (e.g. CEC-exosomes containing or enriched with miR-19a, miR-21, and miR-146a) will not attenuate the proteins increased by patient-clot-derived exosomes. Collectively, these in vivo and in vitro data provide strong cause-effect evidence to demonstrate that the three miRNAs delivered by CEC-exosomes play an important role in mediating therapeutic effect of naïve CEC-exosomes. Based on bioinformatics analysis data showing these three miRNAs have interactive roles in suppressing pro-thrombotic proteins, the inventors thus select to reduce all of them. The inventors are aware that suppressing these miRNAs in endothelial cells by siRNAs may substantially induce cell dysfunction. If so, the inventors will individually attenuate these miRNAs and reexamine their interactive roles in suppressing pro-thrombotic proteins.

Experiment 3b: To examine whether tailored CEC-exosomes carrying elevated amounts of miR-21 or miR-146a further improves the therapeutic effect of naïve CEC-exosomes. To generate CEC-exosomes with the elevated miRNAs, cerebral endothelial cells harvested from young adult male rats will be transfected with mimics of miR-19a, miR-21, or miR-146a by means of the electroporation. Cells transfected with scramble RNAs will be used as a control group. Exosomes will be isolated from supernatants of these cultured cerebral endothelial cells, as demonstrated in the preliminary data (as shown in FIG. 2). Elevation of miR-19a, miR-21, and miR-146a within CEC-exosomes is verified with qRT-PCR prior to in vivo administration. Aged male rats are subjected to embolic MCAO. Thirty min after MCAO, the Longa scores are performed to assess neurological severity prior to the treatment. CEC-exosomes at a dose determined in the above examples in combination with tPA treatments are initiated via a tail vein 2 h after MCAO and a second dose of CEC-exosomes is administered (IV) 24 h after MCAO. Rats are assigned to the following groups according to a pre-generated randomization schema: 1) CEC-exosomes (elevated levels of +miR-19a, +miR-21, and +miR-146a)/tPA, 2) CEC-exosomes/Scr/tPA, 3) naive CEC-exosomes/tPA, or 4) saline. An array of behavioral tests are performed, as outlined in the above examples. All animals are sacrificed 1 week after MCAO and infarct volume and hemorrhagic areas are measured as outlined in the experimental examples described above.

Without wishing to be bound by any particular theory, based on the preliminary data, it is expected that tailored CEC-exosomes with elevated levels miR-19a, miR-21, and miR-146a will robustly reduce infarct volume and improve neurological outcome compared to the scramble-CEC-exosomes and naïve-CEC-exosomes groups. Because exosomes natively transport biological information between cells, exosomes are well-suited to delivery of therapeutic molecules. Thus, the expected results from the proposed experimental procedures outlined in this example, will show that these engineered exosomes provide a more potent and effective therapy when compared to naïve CEC-exosomes. The inventors have previously engineered mesenchymal stromal cell derived exosomes carrying elevated or reduced miRNAs for treatment of ischemic stroke 1, 39. Should the Exp 3a-1 and 3a-2 procedures fail to demonstrate that these three miRNAs evoke the therapeutic effect of CEC-exosomes, other miRNAs than these three miRNAs will be selected for enriching the CEC-exosomes described herein and determine whether other specific microRNAs may be used to selectively regulate cerebral vascular inflammation and thrombosis. These additional candidate miRNAs can include the miR-17-92 cluster in which individual members regulate cerebral vascular inflammation and thrombosis.

C3 Statistical Consideration: This is a study of CEC-exosome therapy as an adjunctive treatment to enhance tPA and thrombectomy treatment of acute ischemic stroke. The proposed experiments consist of male and female rats and sex will be included in all the analysis as a stratification variable. The primary endpoint is the reduction of neurovascular damage with the expectation of possible synergistic effects of the adjuvant therapy on facilitation of ischemic tissue reperfusion and on improvement of neurological outcome. To study synergistic effects of CEC-exosomes (A) in combination with tPA (B) or CEC-exosomes (A) immediately following reperfusion after transient MCAO (B), the inventors consider a complete 2×2 factorial design with all combinations of treatments in 4 groups (A alone, B alone, no A or B and A+B) and ANOVA or ANCOVA if there is repeated outcome assessments (functional tests or MRI measurements over time). The analysis begins with the testing for A and B interaction, following by assessment of super-additive/sub-additive effects of treatment A and B if the interaction is detected at the criteria of 0.05. The super-additive effects of adjuvant therapy indicates that the effect of combined therapy is superior (in term of infarction reduction) to the additive effects from both A alone and B alone, therefore suggesting synergistic effects, which is what the inventors expect for lesion control. This analysis approach can be extended to have the third factor (e.g., sex) in all Aims and also can be used to study pathway factors of the treatments (miR-19a, miR-21, and miR-146a) in the Examples provided above. In addition, to study the effect of the treatment and MRI indices on recanalization and tissue perfusion to predict reduction of infarction, a mixed regression model is considered with inclusion of the combination treatments and MRI indices as covariates and infarction size (percentage) as outcome of interest. The model begins testing for the individual covariate effect or covariate by treatment or sex interaction, followed by multivariable modeling. The final multivariable model will include covariate(s) or covariate by treatment/sex interaction with p<0.05 with estimation the model goodness of fit.

Sample size/power calculation: This is a proof-of-concept study and animals will be shared among treatments. The preliminary data showed effect size of 6.26 and 0.99 on 2 and 7 day infarction, respectively, between tPA alone and tPA in combination with CEC-exosomes. For each sex, considering alpha=0.05, a two-sided test, 9/group for 28 day infarction, and 6/group for earlier day assessments, 4 groups (2×2 groups for combination treatment or pathway study) in Aims 1-3, the inventors will have 80% power to detect those effect sizes, assuming equal space of means. Single sex, will be used in the subsequent Aims if there is no difference between male and female groups in the above recited examples. With a pool of 48 (2×2×2 groups 6 sex) or 54 (2×2×2, 9 per group) assuming equal correlation among groups, the inventors have over 80% to detect a significant correlation between MRI measurements and 28 days infarction if the observed correlation coefficient, is at least 0.40 or 0.38. The inventors should have sufficient samples to fully address their hypotheses.

Methods

Models of MCAO: Rectal temperature is kept at 37° C.±0.5° C. during surgical procedure. The physiological variables of mean arterial blood pressure, arterial pH, PO₂, PCO₂ are measured before ischemia and after CEC-exosome administration. 1) Embolic MCAO, aged Wistar rats (18 months) are subjected to embolic MCAO by placement of a 24-hour-old allogeneic clot at the origin of the MCA via an intravascular catheter. 2) Transient MCAO by a filament, aged rats will be subjected to transient (2 h) MCAO by a coated nylon filament as previously described in Jiang Q, Zhang Z G, Chopp M, Helpern J A, Ordidge R J, Garcia J H, et al. Temporal evolution and spatial distribution of the diffusion constant of water in rat brain after transient middle cerebral artery occlusion. J Neurol Sci. 1993; 120(2):123-30; Chopp M, Zhang R L, Chen H, Li Y, Jiang N, Rusche J R. Postischemic administration of an anti-Mac-1 antibody reduces ischemic cell damage after transient middle cerebral artery occlusion in rats. Stroke. 1994; 25(4):869-75; Zhang Z G, Reif D, Macdonald J, Tang W X, Kamp D K, Gentile R J, et al. ARL 17477, a potent and selective neuronal NOS inhibitor decreases infarct volume after transient middle cerebral artery occlusion in rats. J Cereb Blood Flow Metab. 1996; 16(4):599-604; Zhang Z G, Zhang L, Jiang Q, Chopp M. Bone marrow-derived endothelial progenitor cells participate in cerebral neovascularization after focal cerebral ischemia in the adult mouse. Circ Res. 2002; 90(3):284-8; and Zhang Z, Davies K, Prostak J, Fenstermacher J, Chopp M. Quantitation of microvascular plasma perfusion and neuronal microtubule-associated protein in ischemic mouse brain by laser-scanning confocal microscopy. J Cereb Blood Flow Metab. 1999; 19(1):68-78, (the disclosures of which are incorporated herein by reference in their entireties).

Behavioral tests: A baseline of neurological deficits before the treatment will be assayed 30 min after MCAO by means of the Longa five point score that is a simple but reliable method for rapid evaluation of neurological deficits for acute stroke. Rats with a score of 1 and above will be enrolled into experimental groups. An array of behavioral tests including adhesive removable test, foot-fault test, and modified mNSS will be performed. These tests have been well established in the inventor's laboratory, and are sensitive and reliable indices of sensorimotor impairments in the rat following MCAO.

MRI: MRI measurements will be performed before and after the treatment to detect ischemic damage, recanalization of occluded MCA, CBF, and BBB leakage using ADC, T2, MRA, CBF, and vascular permeability (Ki), respectively as described in methods provided in Ding G, Jiang Q, Li L, Zhang L, Zhang Z G, Panda S, et al. MRI of combination treatment of embolic stroke in rat with rtPA and atorvastatin. J Neurol Sci. 2006; 246(1-2):139-47; Jiang Q, Ewing J R, Ding G L, Zhang L, Zhang Z G, Li L, et al. Quantitative evaluation of BBB permeability after embolic stroke in rat using MRI. J Cereb Blood Flow Metab. 2005; 25(5):583-92; Jiang Q, Zhang Z G, Ding G L, Silver B, Zhang L, Meng H, et al. MRI detects white matter reorganization after neural progenitor cell treatment of stroke. Neuroimage. 2006; 32(3):1080-9; and Jiang Q, Zhang Z G, Ding G L, Zhang L, Ewing J R, Wang L, et al. Investigation of neural progenitor cell induced angiogenesis after embolic stroke in rat using MRI. Neuroimage. 2005; 28(3):698-707; the disclosures of which are incorporated herein by reference in their entireties. MRI measurements on the rat are performed in accordance with the inventors previously described methods, see for example, Ding G, Zhang Z, Chopp M, Li L, Zhang L, Li Q, et al. MRI evaluation of BBB disruption after adjuvant AcSDKP treatment of stroke with tPA in rat. Neuroscience. 2014; 271:1-8; Ding G, Jiang Q, Li L, Zhang L, Zhang Z G, Panda S, et al. MRI of combination treatment of embolic stroke in rat with rtPA and atorvastatin. J Neurol Sci. 2006; 246(1-2):139-47; Jiang Q, Ewing J R, Ding G L, Zhang L, Zhang Z G, Li L, et al. Quantitative evaluation of BBB permeability after embolic stroke in rat using MRI. J Cereb Blood Flow Metab. 2005; 25(5):583-92; Jiang Q, Zhang Z G, Ding G L, Silver B, Zhang L, Meng H, et al. MRI detects white matter reorganization after neural progenitor cell treatment of stroke. Neuroimage. 2006; 32(3):1080-9; and Jiang Q, Zhang Z G, Ding G L, Zhang L, Ewing J R, Wang L, et al. Investigation of neural progenitor cell induced angiogenesis after embolic stroke in rat using MRI. Neuroimage. 2005; 28(3):698-707; Jiang Q, Zhang Z G, Ding G L, Zhang L, Ewing J R, Wang L, et al. Investigation of neural progenitor cell induced angiogenesis after embolic stroke in rat using MRI. Neuroimage. 2005; 28(3):698-707; Zhang Z G, Zhang L, Ding G, Jiang Q, Zhang R L, Zhang X, et al. A model of mini-embolic stroke offers measurements of the neurovascular unit response in the living mouse. Stroke. 2005; 36(12):2701-4; Jiang Q, Zhang R L, Zhang Z G, Ewing J R, Divine G W, Chopp M. Diffusion-, T2-, and perfusion-weighted nuclear magnetic resonance imaging of middle cerebral artery embolic stroke and recombinant tissue plasminogen activator intervention in the rat. J Cereb Blood Flow Metab. 1998; 18(7):758-67; and Jiang Q, Chopp M, Zhang Z G, Knight R A, Jacobs M, Windham J P, et al. The temporal evolution of MRI tissue signatures after transient middle cerebral artery occlusion in rat. J Neurol Sci. 1997; 145:15-23, the disclosures of which are incorporated herein by reference in their entireties.

Primary cerebral endothelial cell culture and exosomes isolation: Primary rat cerebral endothelial cells are harvested from young adult male rats in accordance with established methods (as described in Zhang L, Chopp M, Teng H, Ding G, Jiang Q, Yang X P, et al. Combination treatment with N-acetyl-seryl-aspartyl-lysyl-proline and tissue plasminogen activator provides potent neuroprotection in rats after stroke. Stroke. 2014; 45(4):1108-14; Teng H, Zhang Z G, Wang L, Zhang R L, Zhang L, Morris D, et al. Coupling of angiogenesis and neurogenesis in cultured endothelial cells and neural progenitor cells after stroke. J Cereb Blood Flow Metab. 2008; 28(4):764-71; Teng H, Chopp M, Hozeska-Solgot A, Shen L, Lu M, Tang C, et al. Tissue plasminogen activator and plasminogen activator inhibitor 1 contribute to sonic hedgehog-induced in vitro cerebral angiogenesis. PLoS ONE. 2012; 7(3):e33444; and Wang L, Chopp M, Teng H, Bolz M, Francisco M A, Aluigi D M, et al. Tumor necrosis factor alpha primes cerebral endothelial cells for erythropoietin-induced angiogenesis. J Cereb Blood Flow Metab. 2011; 31(2):640-7, the disclosures of which are incorporated herein by reference in their entireties), and are cultured in exosome free media. Exosomes are isolated from the supernatant of cultured endothelial cells according to published protocols described in Xin H, Li Y, Cui Y, Yang J J, Zhang Z G, Chopp M. Systemic administration of exosomes released from mesenchymal stromal cells promote functional recovery and neurovascular plasticity after stroke in rats. J Cereb Blood Flow Metab. 2013; 33(11):1711-5; and Xin H, Li Y, Buller B, Katakowski M, Zhang Y, Wang X, et al. Exosome-mediated transfer of miR-133b from multipotent mesenchymal stromal cells to neural cells contributes to neurite outgrowth. Stem Cells. 2012; 30(7):1556-64, the disclosures of which are incorporated herein by reference in their entireties. Briefly, the supernatant is filtered via a 0.22 μm filter (Millipore, CA) to remove dead cells and large growth debris, followed by a centrifugation step comprising centrifuging at 10,000×g for 30 minutes to further eliminate small debris. A 100,000×g centrifugation step for 3 hours is performed to collect the exosome pellet. The exosomes will be re-suspended in sterile PBS and quantified (numbers and size of the exosome particles) with a qNano system (IZON, UK). The quantified exosomes are administered to recipient within 1 h after isolation.

### Example 5. Intra-Arterial Administration of Healthy CEC-Exosomes Reduces Infarct Volume after Transient Middle Cerebral Artery Occlusion (MCAO)

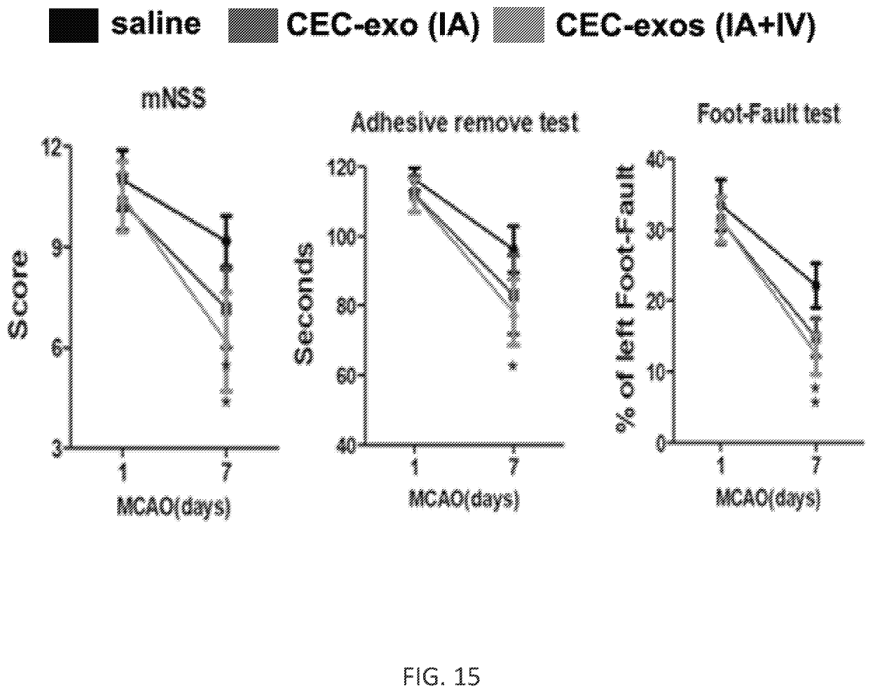

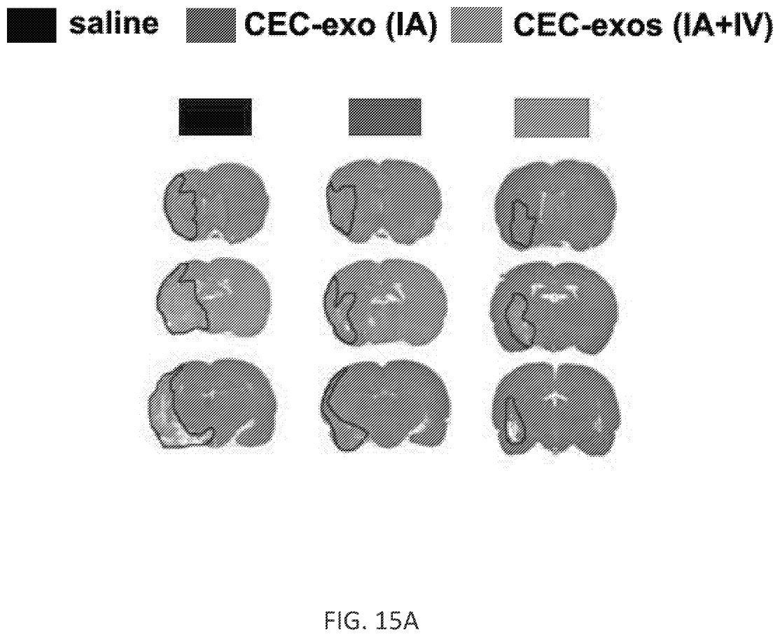

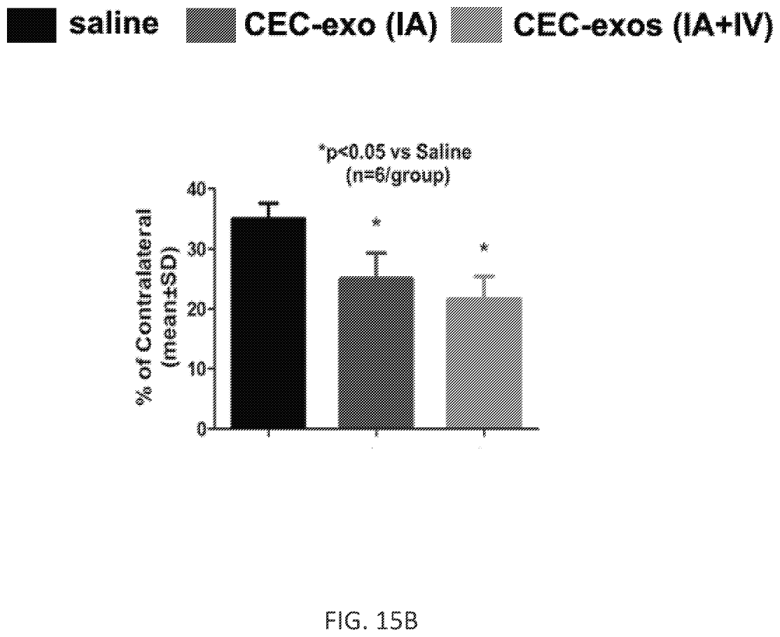

To mimic thrombectomy, a model of transient MCAO was employed. Briefly, the right middle cerebral artery (MCA) was occluded by a filament for 2 hours and the filament was then withdrawn. Immediately the removal of the filament, rats were randomly treated with intracarotid artery injection of CEC-exos (1×10¹¹ particles/injection, CEC-exos+IA), or the same volume of saline (saline). Some ischemic rats received a second dose of CEC-exos (1×10¹¹ particles/injection, CEC-exos+IA+IV) 24 hours after transient MCAO via a tail vein (IV). The inventors have determined that both CEC-exos+IA (n=6) and CEC-exos+IA+IV (n=6) significantly reduced infarct volume (FIGS. 15A and B) and improved neurological outcome (FIG. 15C) compared to the saline treatment (n=6) at 7 days after transient MCAO. These data indicate that healthy CEC-exos have a therapeutic effect on acute stroke, suggesting that this approach can be applied to thrombectomy for further enhancement of neuroprotection.

### Example 6. Intra-Arterial Administration of Human Stroke Patient Derived Exosomes Exacerbates Infarct Volume after Transient MCAO

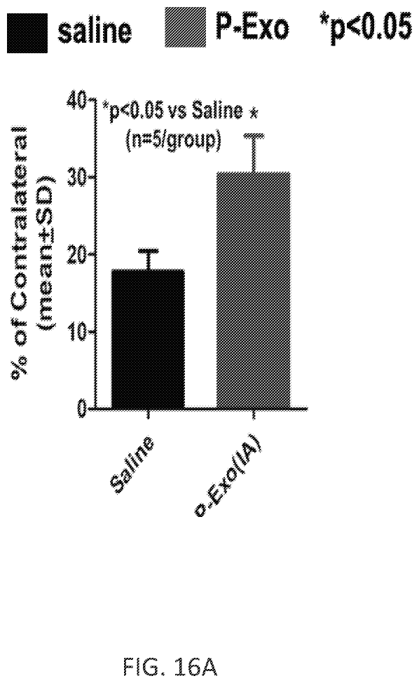

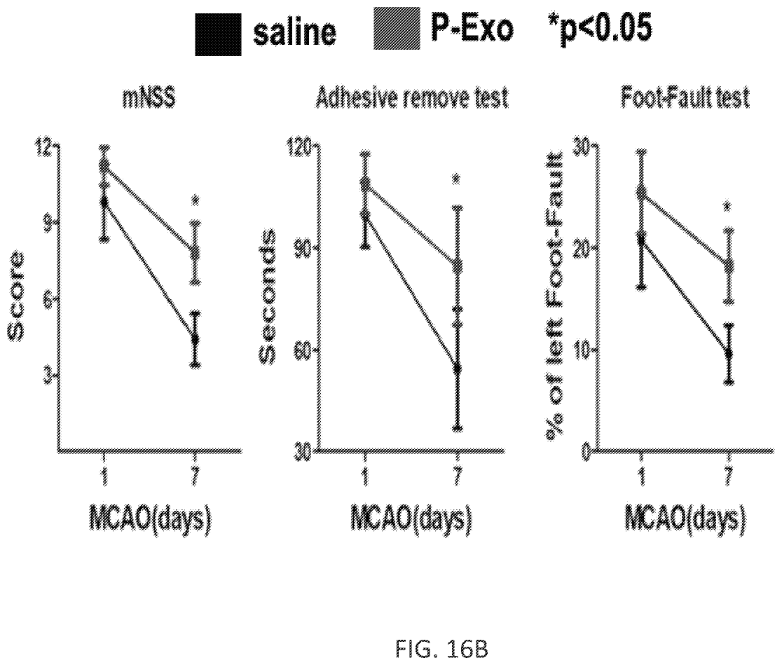

To test the hypothesis that exosomes released by clot-injured cerebral endothelial cells exacerbate infarction after thrombectomy, the inventors administered human stroke patient-derived exosomes to rats subjected to 1 hour tMCAO. One hour of MCAO induces a smaller volume of cerebral infarction than 2 hours of tMCAO. Exosomes were isolated from arterial blood samples collected during thrombectomy of patients with acute ischemic stroke. Young adult male rats were subjected to 1 hour tMCAO by a filament. Immediately upon withdrawing the filament, rats were randomly treated with patient-derived exosomes (1×10¹¹ particles/injection, P-exos, n=5), or the same volume of saline (saline, n=5) via the internal carotid artery. The inventors found that compared to the saline treatment, patient-derived exosomes significantly enlarged infract volume by approximately 37% (FIG. 16A) and worsened neurological outcome (FIG. 16B). These data suggest that exosomes released by clot-injured cerebral endothelial cells can lead to further damage of the neurovascular unit. These data also provide insight into clinical observations that recanalization achieved by the thrombectomy still results in ˜30% of patients without improvement or worsening of their neurological outcomes.

### Example 7. Increased Inflammatory and Pro-Thrombotic Protein Levels in Exosomes Isolated from Arterial Blood Samples Collected During Thrombectomy of Patients with Acute Ischemic Stroke are Correlated to Reduced Improvement of Patient Neurological Function after Thrombectomy

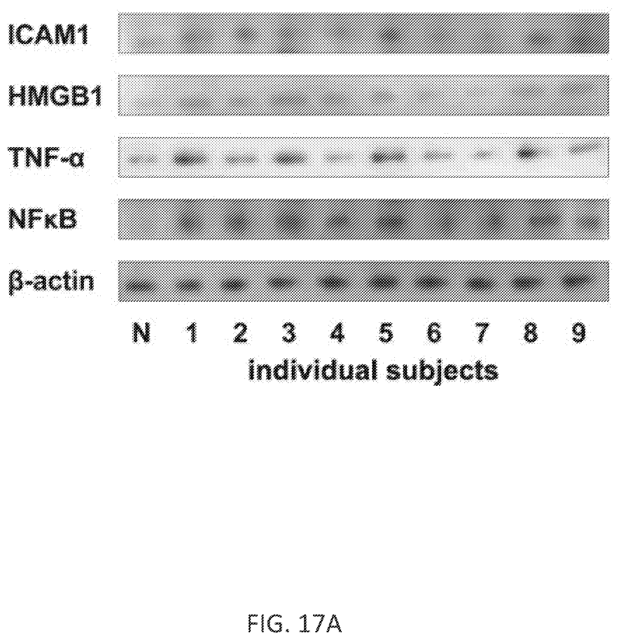

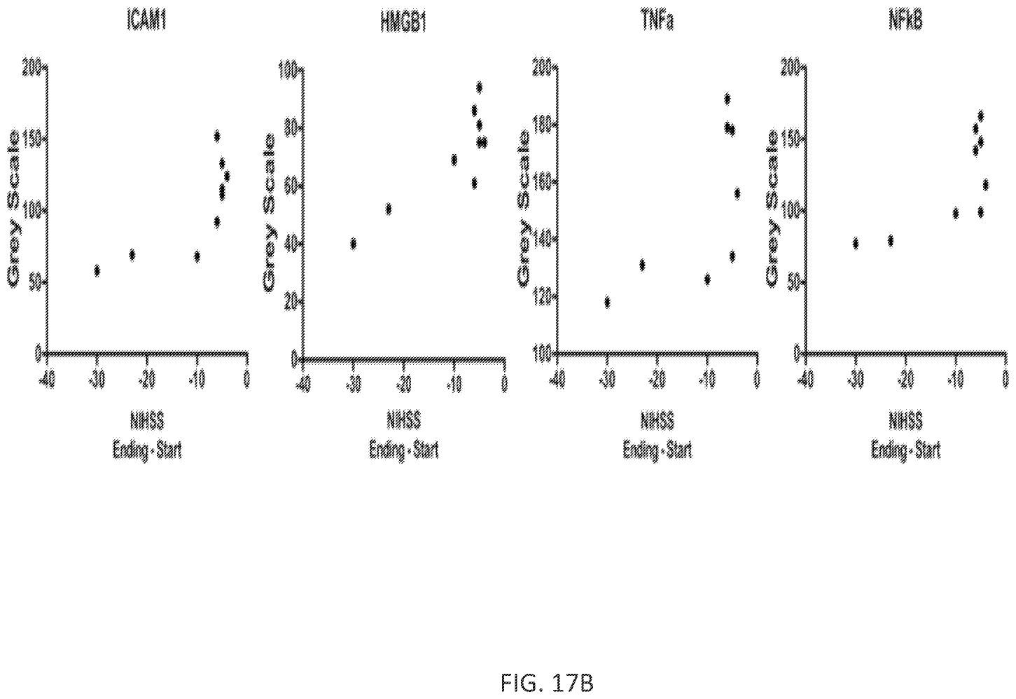

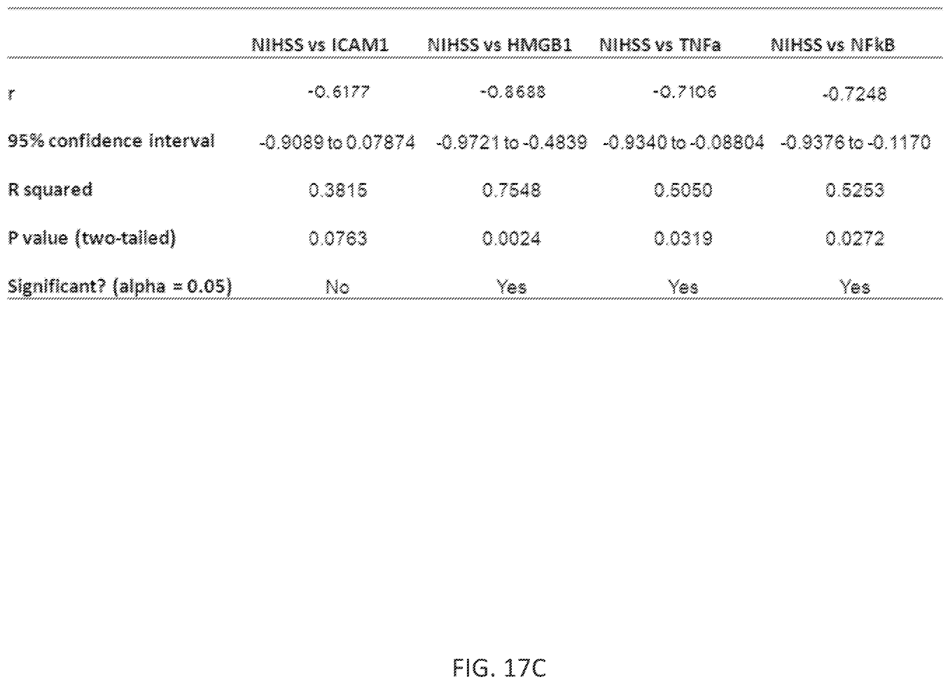

Exosomes were isolated from arterial blood samples collected during thrombectomy of patients with acute ischemic stroke and then measured selected exosomal proteins by means of Western blot. The selected proteins are involved in inflammation and thrombosis. Using NIH stroke scores obtained prior to the thrombectomy and at discharge of individual patients, the inventors correlated NIH stroke scores with exosomal protein levels. The inventors found that there was an inverse and significant correlation between levels of these exosomal proteins and improvement of neurological function at discharge (FIG. 17A-17C). These data suggest that exosomal cargo proteins may be used as potential biomarkers to predict patient functional outcome after thrombectomy. These results also provide additional insight into the adverse effects of stroke generated vascular exosomes on inducing secondary thrombosis and evolving neurovascular damage.

Methods to Isolate Plasma Exosomes from Arterial Blood Samples

Arterial blood samples were collected into plasma separator tubes and then were processed to collect plasma using centrifuge at 3,000 g for 10 min. Plasma were filtered using a 0.2 m syringe filter. After that, filtered plasma were processed by means of differential ultracentrifugation to acquire exosomes. Western blot, transmission electron microscope, and qNano particle analysis were performed to confirm exosomes.

Arterial blood samples were acquired from a cerebral artery during thrombectomy of patients with acute ischemic stroke

Discussion

Based on preclinical data, the inventors expect that administration of exosomes derived from healthy human cerebral endothelial cells (healthy CEC-exosomes) to patients with acute stroke will prevent ischemic lesion expansion by inhibiting cerebral vascular secondary thrombosis and by suppressing BBB leakage. Moreover, intra cerebral artery (IA) administration of healthy CEC-exosomes immediately follow thrombectomy will further enhance cerebral vascular perfusion and BBB integrity, consequently reducing neurovascular damage and improvement of neurological function.

### Example 8. Stroke Patient-Derived Exosomes Promote BBB Leakage of Healthy Cerebral Endothelial Cells, which can be Blocked by CEC-Exosomes

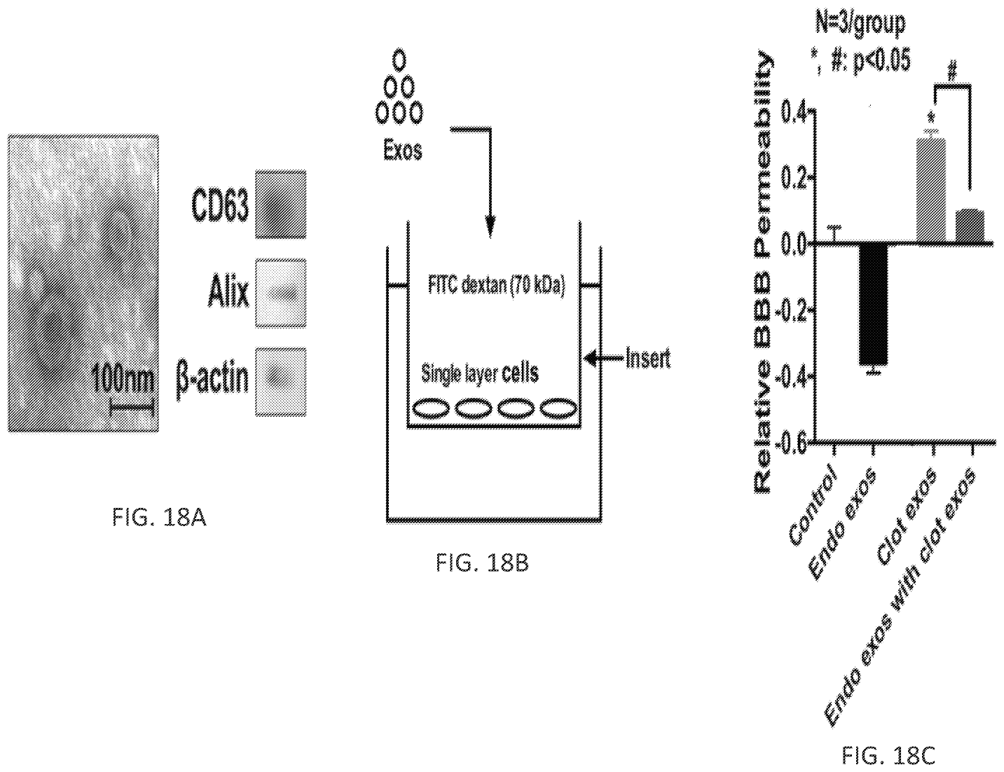

The inventors isolated exosomes from thrombectomy-retrieved thrombi and arterial blood from elderly patients with acute stroke. TEM revealed that these exosomes had doughnut morphology and mean diameter ˜100 nm (FIG. 18A). Western blot analysis showed the presence of CD63 and Alix (FIG. 18A). Thus, these are exosomes, which differ from microparticles that are released from the plasma membrane during budding or shedding and have diameter range from 0.1 to 1 μm. Parent cells for derived exosomes likely include blood cells, platelets and endothelial cells. Using an in vitro BBB permeability assay, the inventors first examined the effect of stroke patient derived exosomes on BBB permeability. Healthy human cerebral endothelial cells were seeded in a single layer in the inserted and FITC-dextran was added into the inserted well (FIG. 18B). By quantifying FITC-dextran signal in the main well, the inventors were able to measure BBB leakage (FIG. 18B). Incubation of healthy human cerebral endothelial cells with patient-derived exosomes increased BBB leakage measured by an in vitro BBB method) showing an increase of FITC-dextran crossing the cell layer (FIG. 18C). However, application of CEC-exos significantly BBB leakage induced by stroke patient derived exosomes. Moreover, CEC-exos significantly reduced BBB leakage compared to the control group (FIG. 18C). These data for the first time provide evidence that stroke patient-derived exosomes promote BBB leakage. More importantly, CEC-exos can suppress patient-exosome-promoted BBB leakage.

### Example 9. Tailored CEC-Exosomes or MSC-Exosomes with Elevated miR-146a have a Superior Effect on Reduction of BBB Leakage Compared to Naïve CEC-Exosomes or MSC-Exosomes

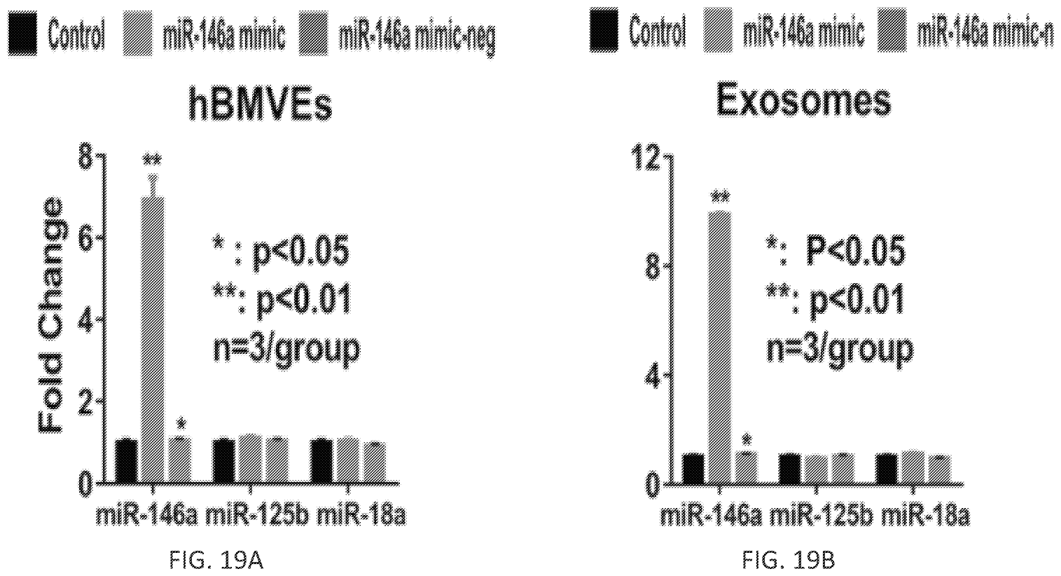

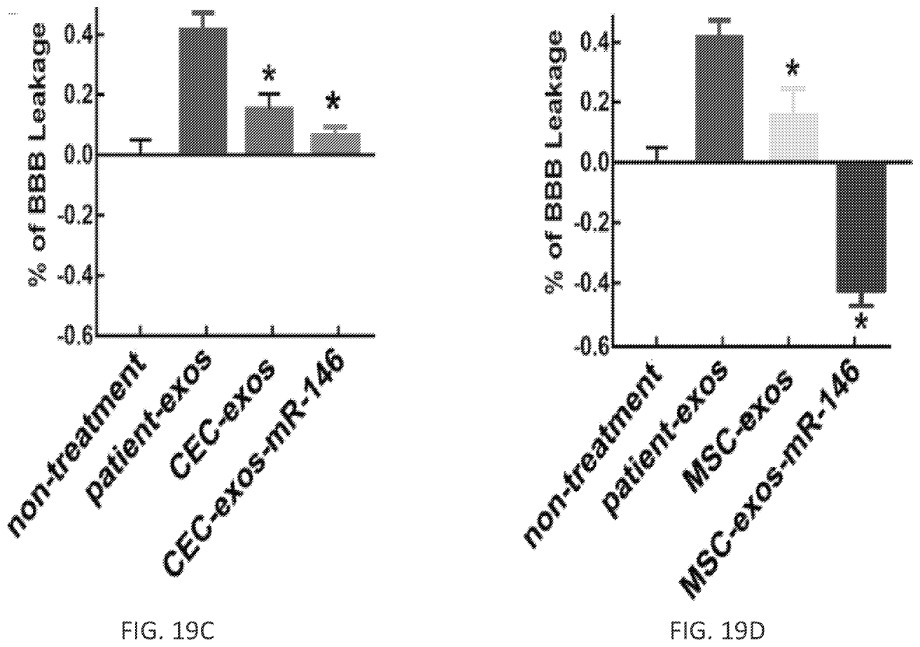

To examine the effect of the tailored CEC-exosomes carrying elevated miR-146a on BBB leakage, the inventors transfected with CECs with miR-146a mimics (System Biosciences, Cat #XMIR-146a) or mimic control (System Biosciences, Cat #XMIR-POS) and then isolated exosomes from supernatants of the transfected cells. Quantitative RT-PCR analysis showed that compared to control without transfection and control with transfection of mimic negative, transfection of miR-146a mimics elevated ˜8 fold and ˜10 fold of miR-146a in CECs (FIG. 19A) and CEC-exosomes (FIG. 19B), respectively, but did not affect other miRNAs, such as miR-125b and -18a, which are present in CECs and CEC-exosomes (FIG. 19A, B), suggesting that elevation of miR-146a in the CECs and CEC-exosomes is specific. Using the in vitro BBB permeability assay, the inventors then examined the effect of the miR-146a tailored CEC-exosomes on BBB leakage. The inventors found that naïve CEC-exosomes significantly reduced stroke patient-exosomes increased BBB leakage, whereas CEC-exosomes carrying elevated miR-146a further significantly reduced BBB leakage compared to naïve CEC-exosomes (FIG. 19C). MSC-exosomes also reduced patient-exosomes increased BBB leakage and MSC-exosomes carrying elevated miR-146a robustly blocked patient-exosomes-increased BBB leakage (FIG. 19D). Together, these data indicate that tailored exosomes carrying elevated miR-146a are more potent to reduce BBB leakage.

While some embodiments have been particularly shown and described with reference to the foregoing preferred and alternative embodiments, it should be understood by those skilled in the art that various alternatives to the embodiments described herein may be employed in practicing the invention without departing from the spirit and scope of the invention as defined in the following claims. It is intended that the following claims define the scope of the invention and that the methods, systems, and compositions within the scope of these claims and their equivalents be covered thereby. This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A combination comprising mammalian exosomes and Tissue Plasminogen Activator (tPA) for use in the treatment of stroke, wherein the combination of mammalian exosomes and tPA is for administration to a subject in need thereof in a therapeutically effective amount.

2. A combination comprising mammalian exosomes and tPA for use in the treatment or prevention of cerebrovascular injury, wherein the combination of mammalian exosomes and tPA is for administration to a subject in need thereof in a therapeutically effective amount.

3. Mammalian exosomes for use in the treatment of stroke in a subject in need thereof, wherein the treatment further comprises performing a thrombectomy and wherein the mammalian exosomes are for admiration to the subject in a therapeutically effective amount.

4. A combination comprising mammalian exosomes and tPA for use in the treatment or prevention of secondary thrombosis in downstream brain microvessels, wherein the combination of mammalian exosomes and tPA is for administration to a subject in need thereof in a therapeutically effective amount.

5. A combination comprising mammalian exosomes and tPA for use in the treatment or prevention of blood brain barrier impairment, wherein the combination of mammalian exosomes and tPA is for administration to a subject in need thereof in a therapeutically effective amount.

6. A combination comprising mammalian exosomes and tPA for use in the treatment or prevention of blood brain barrier leakage, wherein the combination of mammalian exosomes and tPA is for administration to a subject in need thereof in a therapeutically effective amount.

7. A combination for use according to any of the preceding claims, wherein the subject is a subject that has suffered a stroke.

8. A combination for use according to any of the preceding claims, wherein the therapeutically effective amount of the combination provides prevention, amelioration or reduction of a symptom related to cerebrovascular injury.

9. A combination for use according to any of the preceding claims, wherein the cerebrovascular injury is one or more of: neuronal damage, residual clot persistence, microvascular hypoperfusion, blood-brain-barrier leakage, and ischemic lesion expansion.

10. A combination for use according to any of the preceding claims, wherein the subject is a human.

11. A combination for use according to any of the preceding claims, wherein the stroke is an ischemic stroke.

12. A combination for use according to any of the preceding claims, wherein a therapeutically effective amount of the mammalian exosomes ranges from 0.0001 μg/kg to 1.0 mg/kg the subject's body weight.

13. A combination for use according to any of the preceding claims, wherein the therapeutically effective amount of the mammalian exosomes ranges from 0.0007 μg/kg to 7.0 mg/kg the subject's body weight.

14. A combination for use according to any of the preceding claims, wherein the therapeutically effective amount of tPA ranges from 0.6 mg/kg to 7.0 mg/kg the subject's body weight.

15. A combination for use according to any of the preceding claims, wherein the therapeutically effective amount of tPA ranges from 0.6 mg/kg to 1.0 mg/kg the subject's body weight.

16. A combination for use according to any of the preceding claims, wherein the mammalian exosomes is an exosome containing at least one of the miRNAs miRNA-19a, miRNA-21, or miRNA-146a.

17. A combination for use according to any of the preceding claims, wherein the miRNA-146a is selectively overexpressed in the mammalian exosome over the level of miRNA-146a expression in naïve or control exosomes.

18. A combination for use according to any of the preceding claims, wherein the mammalian exosomes are enriched with miR-146a.

19. A combination for use according to any of the preceding claims, wherein the concentration of miR-146a in the mammalian exosomes is at least about twice the concentration of miR-146a in naïve or control exosomes.

20. A combination for use according to any of the preceding claims, wherein the concentration of miR-146a in the mammalian exosomes is at least about three times the concentration of miR-146a in naïve or control exosomes.

21. A combination for use according to any of the preceding claims, wherein the concentration of miR-146a in the mammalian exosomes is at least about four times the concentration of miR-146a in naïve or control exosomes.

22. A combination for use according to any of the preceding claims, wherein the concentration of miR-146a in the mammalian exosomes is at least about five times the concentration of miR-146a in naïve or control exosomes.

23. A combination for use according to any of the preceding claims, wherein the concentration of miR-146a in the mammalian exosomes is at least about six times the concentration of miR-146a in naïve or control exosomes.

24. A combination for use according to any of the preceding claims, wherein the concentration of miR-146a in the mammalian exosomes is at least about seven times the concentration of miR-146a in naïve or control exosomes.

25. A combination for use according to any of the preceding claims, wherein the concentration of miR-146a in the mammalian exosomes is at least about eight times the concentration of miR-146a in naïve or control exosomes.

26. A combination for use according to any of the preceding claims, wherein the concentration of miR-146a in the mammalian exosomes is at least about nine times the concentration of miR-146a in naïve or control exosomes.

27. A combination for use according to any of the preceding claims, wherein the concentration of miR-146a in the mammalian exosomes is at least about 10 times the concentration of miR-146a in naïve or control exosomes.

28. A combination for use according to any of the preceding claims, wherein the concentration of miR-146a in the mammalian exosomes is at least about 100 times the concentration of miR-146a in naïve or control exosomes.

29. A combination for use according to any of the preceding claims, wherein the concentration of miR-146a in the mammalian exosomes is at least about 1000 times the concentration of miR-146a in naïve or control exosomes.

30. A combination for use according to any of the preceding claims, wherein the mammalian exosomes are derived or isolated from stem cells, mesenchymal stromal cells, umbilical cord cells, endothelial cells, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes.

31. A combination for use according to any of the preceding claims, wherein a therapeutically effective amount of the mammalian exosomes comprises from about 1×10⁷ to about 1×10¹⁷ exosomes.

32. A combination for use according to any of the preceding claims, wherein the therapeutically effective amount of mammalian exosomes comprises from about 1×10¹² to about 1×10¹⁵ exosomes.

33. A combination for use according to any of the preceding claims, wherein the mammalian exosomes are administered by intravenous injection, intra-arterial injection, subcutaneous injection, intramuscular injection, intraperitoneally, stereotactically, intranasally, mucosally, intravitreally, intrastriatally, or intrathecally.

34. A combination for use according to any of the preceding claims, wherein the mammalian exosomes are administered by intravenous injection.

35. A combination for use according to any of the preceding claims, wherein the therapeutically effective amount of a combination of mammalian exosomes and tPA are administered after the onset of stroke symptoms.

36. A combination for use according to any of the preceding claims, wherein the mammalian exosomes are administered after the onset of stroke symptoms.

37. A combination for use according to any of the preceding claims, wherein the mammalian exosomes are administered 1 minute to 9 hours after the onset of stroke symptoms.

38. A combination for use according to any of the preceding claims, wherein the mammalian exosomes are administered about 10 minutes to about 6 hours after the occurrence of stroke.

39. A combination for use according to any of the preceding claims, wherein the mammalian exosomes are administered about 10 minutes to about 12 hours after the occurrence of stroke.

40. A combination for use according to any of the preceding claims, wherein the mammalian exosomes are administered about 10 minutes to about 24 hours after the occurrence of stroke.

41. A combination for use according to any of the preceding claims, wherein the mammalian exosomes are administered about 10 minutes to about 48 hours after the occurrence of stroke.

42. A combination for use according to any of the preceding claims, wherein the mammalian exosomes are administered about 10 minutes to about 36 hours after the occurrence of stroke.

43. A combination for use according to any of the preceding claims, wherein the mammalian exosomes are administered about 10 minutes to about 72 hours after the occurrence of stroke.

44. A combination for use according to any of the preceding claims, wherein the mammalian exosomes are administered about 10 minutes to about 4 days after the occurrence of stroke.

45. A combination for use according to any of the preceding claims, wherein the mammalian exosomes are administered about 10 minutes to about 5 days after the occurrence of stroke.

46. A combination for use according to any of the preceding claims, wherein the mammalian exosomes are administered about 10 minutes to about 6 days after the occurrence of stroke.

47. A combination for use according to any of the preceding claims, wherein the mammalian exosomes are administered about 10 minutes to about 7 days after the occurrence of stroke.

48. A combination for use according to any of the preceding claims, wherein the mammalian exosomes are administered about 10 minutes to about 8 days after the occurrence of stroke.

49. A combination for use according to any of the preceding claims, wherein the mammalian exosomes are administered about 10 minutes to about 9 days after the occurrence of stroke.

50. A combination for use according to any of the preceding claims, wherein the mammalian exosomes are administered about 10 minutes to about 10 days after the occurrence of stroke.

51. A combination for use according to any of the preceding claims, wherein the tPA is administered after the onset of stroke symptoms.

52. A combination for use according to any of the preceding claims, wherein the tPA is administered 1 minute to 9 hours after the onset of stroke symptoms.

53. A combination for use according to any of the preceding claims, wherein the mammalian exosomes and tPA are administered concomitantly or sequentially.

54. A combination for use according to any of the preceding claims, wherein the administration of the mammalian exosomes increases the therapeutic window in which tPA may be administered.

55. A combination for use according to any of the preceding claims, wherein the increase of the therapeutic window in which tPA may be administered after the onset of stroke symptoms is 6 hours to 12 hours.

56. A combination for use according to any of the preceding claims, wherein the administration of the therapeutically effective combination provides one or more therapeutic benefits to the subject treated with the combination: (a) increased proteolysis of fibrin in a clot, (b) extends the therapeutic window beyond 3-4.5 hours for administering tPA (c) increases the rate and extent of vessel recanalization, (d) increases microvascular reperfusion without increased brain hemorrhage, (e) reduces leakage of the blood-brain-barrier, (f) attenuates infarct expansion, (g) reduces prothrombotic procoagulant vascular conditions, (h) reduces vascular and/or cerebral brain cell inflammation, and (i) reduces prothrombotic procoagulant vascular conditions and vascular and subsequent cerebral brain cell inflammation.

57. A combination for use according to any of the preceding claims, wherein the administration of the therapeutically effective combination provides an extension of the therapeutic window for administering tPA to cause a measurable thrombolytic effect in the subject having the stroke.

58. A combination for use according to any of the preceding claims, wherein the thrombectomy is performed with a stent retriever, coil retriever, aspiration device, balloon maceration device, hydrodynamic device, acoustic energy device, spinning brush, or spinning wire device.

59. A combination for use according to any of the preceding claims, wherein the therapeutically effective amount of mammalian exosomes are administered, and the thrombectomy is performed, after the onset of stroke symptoms.

60. A combination for use according to any of the preceding claims, wherein the thrombectomy is performed after the onset of stroke symptoms.

61. A combination for use according to any of the preceding claims, wherein the thrombectomy is performed 1 minute to 24 hours after the onset of stroke symptoms.

62. A combination for use according to any of the preceding claims, wherein the mammalian exosomes are administered, and the thrombectomy is performed, concomitantly or sequentially.

63. A combination for use according to any of the preceding claims, wherein the administration of the therapeutically effective amount of mammalian exosomes and the performance of the thrombectomy in combination provides one or more therapeutic benefits to the subject treated with the combination: (a) increased proteolysis of fibrin in a clot, (b) increases the rate and extent of vessel recanalization, (c) increases microvascular reperfusion without increased brain hemorrhage, (d) reduces leakage of the blood-brain-barrier, and (e) attenuates infarct expansion.

64. A combination for use according to any of the preceding claims, wherein the administration of the therapeutically effective combination provides an extension of the therapeutic window for administering tPA to cause a measurable thrombolytic effect in the subject having the stroke.

65. A combination for use according to any of the preceding claims, wherein the mammalian exosomes containing or enriched with miRNAs miRNA-19a, miRNA-21, or miRNA-146a comprise human endothelial cells, or endothelial cell progenitor cells.

66. A combination for use according to any of the preceding claims, wherein the human endothelial cells comprise primary or tissue cultured cerebral endothelial cells (CEC).

67. A combination for use according to any of the preceding claims, wherein the method further comprises: (a) administration of a therapeutically effective dose of tPA prior to, or subsequent to the administration of the mammalian exosomes, or (b) a thrombectomy procedure performed prior to, or subsequent to the administration of the mammalian exosomes.

68. A composition comprising mammalian exosomes enriched with at least one miRNAs selected from the group consisting of: miRNA-19a, miRNA-21, and miRNA-146a.

69. The composition of claim 68, wherein miRNA-146a is selectively overexpressed in the mammalian exosomes over the level of miRNA-146a expression in naïve or control exosomes.

70. The composition of any of claims 68-69, wherein the mammalian exosomes are human exosomes derived from a human cell culture.

71. The composition of any of claims 68-70, wherein the human exosomes are derived from human endothelial cells, or human endothelial cell progenitor cells.

72. A composition comprising a modified population of cells, wherein the cells overexpress miR-146a over the level of expression of said miRNA-146a in naïve or control cells.

73. The composition of claim 72, wherein the cells have been modified through transient transfection with an miRNA-146a mimic.

74. The composition of any of claims 72-73, wherein the control cells are cells that have been transfected with a mimic control that does not express miRNA-146a.

75. The composition of any of claims 72-74, wherein the cells are human endothelial cells, or human endothelial cell progenitor cells.

76. The composition of any of claims 72-75, wherein the cells overexpress miR-146a at least 2 fold as compared to the level of expression of said miRNA-146a in naïve or control cells.

77. The composition of any of claims 72-76, wherein the cells overexpress miR-146a by at least 3 fold as compared to the level of expression of said miRNA-146a in naïve or control cells.

78. The composition of any of claims 72-77, wherein the cells overexpress miR-146a by at least 5 fold as compared to the level of expression of said miRNA-146a in naïve or control cells.

79. The composition of any of claims 72-78, wherein the cells overexpress miR-146a by at least 10 fold as compared to the level of expression of said miRNA-146a in naïve or control cells.

80. The composition of any of claims 72-79, wherein the cells overexpress miR-146a by at least 5% when compared to the level of expression of said miRNA-146a in naïve or control cells.

81. The composition of any of claims 72-80, wherein the cells overexpress miR-146a by at least 10% when compared to the level of expression of said miRNA-146a in naïve or control cells.

82. The composition of any of claims 72-81, wherein the cells overexpress miR-146a by at least 25% when compared to the level of expression of said miRNA-146a in naïve or control cells.

83. The composition of any of claims 72-82, wherein the cells overexpress miR-146a by at least 50% when compared to the level of expression of said miRNA-146a in naïve or control cells.

84. A composition comprising a plurality of mammalian exosomes, wherein the mammalian exosomes comprise miR-146a.

85. The composition of claim 84, wherein the mammalian exosomes are enriched with miR-146a.

86. The composition of any of claims 84-85, wherein the concentration of miR-146a in the mammalian exosomes is at least about twice the concentration of miR-146a in naïve or control exosomes.

87. The composition of any of claims 84-86, wherein the concentration of miR-146a in the mammalian exosomes is at least about three times the concentration of miR-146a in naïve or control exosomes.

88. The composition of any of claims 84-87, wherein the concentration of miR-146a in the mammalian exosomes is at least about four times the concentration of miR-146a in naïve or control exosomes.

89. The composition of any of claims 84-88, wherein the concentration of miR-146a in the mammalian exosomes is at least about five times the concentration of miR-146a in naïve or control exosomes.

90. The composition of any of claims 84-89, wherein the concentration of miR-146a in the mammalian exosomes is at least about six times the concentration of miR-146a in naïve or control exosomes.

91. The composition of any of claims 84-90, wherein the concentration of miR-146a in the mammalian exosomes is at least about seven times the concentration of miR-146a in naïve or control exosomes.

92. The composition of any of claims 84-91, wherein the concentration of miR-146a in the mammalian exosomes is at least about eight times the concentration of miR-146a in naïve or control exosomes.

93. The composition of any of claims 84-92, wherein the concentration of miR-146a in the mammalian exosomes is at least about nine times the concentration of miR-146a in naïve or control exosomes.

94. The composition of any of claims 84-93, wherein the concentration of miR-146a in the mammalian exosomes is at least about 10 times the concentration of miR-146a in naïve or control exosomes.

95. The composition of any of claims 84-94, wherein the concentration of miR-146a in the mammalian exosomes is at least about 100 times the concentration of miR-146a in naïve or control exosomes.

96. The composition of any of claims 84-95, wherein the concentration of miR-146a in the mammalian exosomes is at least about 1000 times the concentration of miR-146a in naïve or control exosomes.

97. The composition of any of claims 84-96, wherein the mammalian exosomes are derived from a mammalian cell.

98. The composition of any of claims 84-97, wherein the mammalian exosomes are derived or isolated from stem cells, mesenchymal stromal cells, umbilical cord cells, endothelial cells, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes.

99. The composition of any of claims 84-98, wherein the mammalian exosomes are derived from human endothelial cells or human endothelial cell progenitor cells that have been transfected with a miRNA-146a mimic.

100. A composition comprising mammalian exosomes enriched with at least one miRNAs selected from the group consisting of: miRNA-19a, miRNA-21, and miRNA-146a.

101. The composition of any of claims 84-100, wherein the mammalian exosomes overexpress miR-146a by at least 2 fold as compared to the level of expression of said miRNA-146a in naïve or control cells.

102. The composition of any of claims 84-101, wherein the mammalian exosomes overexpress miR-146a by at least 3 fold as compared to the level of expression of said miRNA-146a in naïve or control cells.

103. The composition of any of claims 84-102, wherein the mammalian exosomes overexpress miR-146a by at least 5 fold as compared to the level of expression of said miRNA-146a in naïve or control cells.

104. The composition of any of claims 84-103 wherein the mammalian exosomes overexpress miR-146a by at least 10 fold as compared to the level of expression of said miRNA-146a in naïve or control cells.

105. The composition of any of claims 84-104, wherein the mammalian exosomes overexpress miR-146a by at least 5% when compared to the level of expression of said miRNA-146a in naïve or control cells.

106. The composition of any of claims 84-105, wherein the mammalian exosomes overexpress miR-146a by at least 10% when compared to the level of expression of said miRNA-146a in naïve or control cells.

107. The composition of any of claims 84-106, wherein the mammalian exosomes overexpress miR-146a by at least 25% when compared to the level of expression of said miRNA-146a in naïve or control cells.

108. The composition of any of claims 84-107, wherein the mammalian exosomes overexpress miR-146a by at least 50% when compared to the level of expression of said miRNA-146a in naïve or control cells.

109. A combination for use according to any of claims 1-67, wherein the mammalian exosomes overexpress miR-146a by at least 2 fold as compared to the level of expression of said miRNA-146a in naïve or control cells.

110. A combination for use according to any of claims 1-67, wherein the mammalian exosomes overexpress miR-146a by at least 3 fold as compared to the level of expression of said miRNA-146a in naïve or control cells.

111. A combination for use according to any of claims 1-67, wherein the mammalian exosomes overexpress miR-146a by at least 5 fold as compared to the level of expression of said miRNA-146a in naïve or control cells.

112. A combination for use according to any of claims 1-67, wherein the mammalian exosomes overexpress miR-146a by at least 10 fold as compared to the level of expression of said miRNA-146a in naïve or control cells.

113. A combination for use according to any of claims 1-67, wherein the mammalian exosomes overexpress miR-146a by at least 5% when compared to the level of expression of said miRNA-146a in naïve or control cells.

114. A combination for use according to any of claims 1-67, wherein the mammalian exosomes overexpress miR-146a by at least 10% when compared to the level of expression of said miRNA-146a in naïve or control cells.

115. A combination for use according to any of claims 1-67, wherein the mammalian exosomes overexpress miR-146a by at least 25% when compared to the level of expression of said miRNA-146a in naïve or control cells.

116. A combination for use according to any of claims 1-67, wherein the mammalian exosomes overexpress miR-146a by at least 50% when compared to the level of expression of said miRNA-146a in naïve or control cells.

117. A combination for use according to any of the preceding claims, wherein the mammalian exosomes are enriched with miR-19a.

118. A combination for use according to any of the preceding claims, wherein the concentration of miR-19a in the mammalian exosomes is at least about twice, at least about three time, at least about four times, at least about five times, at least about six times, at least about seven times, at least about eight times, at least about nine times, at least about 10 times, at least about 100 times, at least about 1000 times the concentration of miR-19a in naïve or control exosomes.

119. A composition comprising a modified population of cells, wherein the cells overexpress miR-19a over the level of expression of said miR-19a in naïve or control cells.

120. The composition of any of the preceding claims, wherein the cells have been modified through transient transfection with an miR-19a mimic.

121. The composition of any of the preceding claims, wherein the control cells are cells that have been transfected with a mimic control that does not express miR-19a.

122. The composition of any of the preceding claims, wherein the cells are human endothelial cells, or human endothelial cell progenitor cells.

123. The composition of any of the preceding claims, wherein the cells overexpress miR-19a at least 2 fold, at least 3 fold, at least 5 fold or at least 10 fold as compared to the level of expression of said miRNA-19a in naïve or control cells.

124. The composition of any of the preceding claims, wherein the cells overexpress miR-19a by at least 5%, by at least 10%, by at least 25% or by at least 50% when compared to the level of expression of said miRNA-19a in naïve or control cells.

125. A composition comprising a plurality of mammalian exosomes, wherein the mammalian exosomes comprise miR-19a.

126. The composition of any of the preceding claims, wherein the mammalian exosomes are enriched with miR-19a.

127. The composition of any of the preceding claims, wherein the concentration of miR-19a in the mammalian exosomes is at least about twice, at least about three times, at least about four times, at least about five times, at least about six times, at least about seven times, at least about eight times, at least about nine times, at least about ten times, at least about 100 times, or at least about 1000 times the concentration of miR-19a in naïve or control exosomes.

128. The composition of any of the preceding claims, wherein the mammalian exosomes are derived from a mammalian cell.

129. The composition of any of the preceding claims, wherein the mammalian exosomes are derived from human endothelial cells or human endothelial cell progenitor cells that have been transfected with a miR-19a mimic.

130. A combination for use according to any of the preceding claims, wherein the mammalian exosomes overexpress miR-19a by at least 2 fold, by at least 4 fold, by at least 5 fold or by at least 10 fold as compared to the level of expression of said miR-19a.

131. A combination for use according to any of the preceding claims wherein the mammalian exosomes overexpress miR-19a by at least 5%, by at least 10%, by at least 35% or by at least 50% when compared to the level of expression of said miR-19a in naïve or control cells.

132. A combination for use according to any of the preceding claims, wherein the mammalian exosomes are enriched with miR-21.

133. A combination for use according to any of the preceding claims, wherein the concentration of miR-21 in the mammalian exosomes is at least about twice, at least about three time, at least about four times, at least about five times, at least about six times, at least about seven times, at least about eight times, at least about nine times, at least about 10 times, at least about 100 times, at least about 1000 times the concentration of miR-21 in naïve or control exosomes.

134. A composition comprising a modified population of cells, wherein the cells overexpress miR-21 over the level of expression of said miR-21 in naïve or control cells.

135. The composition of any of the preceding claims wherein the cells have been modified through transient transfection with an miR-21 mimic.

136. The composition of any of the preceding claims, wherein the control cells are cells that have been transfected with a mimic control that does not express miR-21.

137. The composition of any of the preceding claims, wherein the cells are human endothelial cells, or human endothelial cell progenitor cells.

138. The composition of any of the preceding claims, wherein the cells overexpress miR-21 at least 2 fold, at least 3 fold, at least 5 fold or at least 10 fold as compared to the level of expression of said miR-21 in naïve or control cells.

139. The composition of any of the preceding claims, wherein the cells overexpress miR-21 by at least 5%, by at least 10%, by at least 25% or by at least 50% when compared to the level of expression of said miR-21 in naïve or control cells.

140. A composition comprising a plurality of mammalian exosomes, wherein the mammalian exosomes comprise miR-21.

141. The composition of any of the preceding claims, wherein the mammalian exosomes are enriched with miR-21.

142. The composition of any of the preceding claims, wherein the concentration of miR-21 in the mammalian exosomes is at least about twice, at least about three times, at least about four times, at least about five times, at least about six times, at least about seven times, at least about eight times, at least about nine times, at least about ten times, at least about 100 times, or at least about 1000 times the concentration of miR-21 in naïve or control exosomes.

143. The composition of any of the preceding claims, wherein the mammalian exosomes are derived from a mammalian cell.

144. The composition of any of the preceding claims wherein the mammalian exosomes are derived from human endothelial cells or human endothelial cell progenitor cells that have been transfected with a miR-21 mimic.

145. A combination for use according to any of the preceding claims, wherein the mammalian exosomes overexpress miR-21 by at least 2 fold, by at least 4 fold, by at least 5 fold or by at least 10 fold as compared to the level of expression of said miRNA-21.

146. A combination for use according to any of the preceding claims, wherein the mammalian exosomes overexpress miR-21 by at least 5%, by at least 10%, by at least 35% or by at least 50% when compared to the level of expression of said miR-21 in naïve or control cells.

147. The composition of any of the preceding claims wherein the mammalian exosomes are derived or isolated from stem cells, mesenchymal stromal cells, umbilical cord cells, endothelial cells, cerebral endothelial cells, epithelial cells, Schwann cells, hematopoietic cells, reticulocytes, monocyte-derived dendritic cells (MDDCs), monocytes, B lymphocytes, antigen-presenting cells, glial cells, astrocytes, neurons, oligodendrocytes, spindle neurons, microglia, or mastocytes.

148. A kit comprising at least one therapeutically effective dose of mammalian exosomes of any of the preceding claims, at least one therapeutically effective dose of tPA, and a package insert comprising instructions for using the mammalian exosomes and tPA in combination to treat or prevent stroke in a subject in need thereof.

149. A kit comprising at least one therapeutically effective dose of mammalian exosomes of any of the preceding claims, at least one therapeutically effective dose of tPA, and a package insert comprising instructions for using the mammalian exosomes and tPA in combination to treat or prevent a cerebrovascular injury in a subject in need thereof.

150. A kit comprising at least one therapeutically effective dose of mammalian exosomes of any of the preceding claims, at least on therapeutically effective dose of tPA, and a package insert comprising instructions for using the mammalian exosomes and tPA in combination to treat or prevent secondary thrombosis in downstream brain microvessels in a subject.

151. A kit comprising at least one therapeutically effective dose of mammalian exosomes of any of the preceding claims, at least one therapeutically effective dose of tPA, and a package insert comprising instructions for using the mammalian exosomes and tPA in combination to treat or prevent a blood brain barrier impairment in a subject.

152. A kit comprising at least one therapeutically effective dose of mammalian exosomes of any of the preceding claims, at least one therapeutically effective dose of tPA, and a package insert comprising instructions for using the mammalian exosomes and tPA in combination to treat or prevent a cerebrovascular injury.

153. The kit of any of the preceding claims, wherein the cerebrovascular injury is neuronal damage, residual clot persistence, microvascular hypoperfusion, blood-brain-barrier leakage, or ischemic lesion expansion.

154. The kit of any of the preceding claims, wherein the cerebrovascular injury is the presentation of symptoms consistent with is neuronal damage, residual clot persistence, microvascular hypoperfusion, blood-brain-barrier leakage, or ischemic lesion expansion.

155. A kit comprising at least one therapeutically effective dose of mammalian exosomes of any of the preceding claims, at least one therapeutically effective dose of tPA, at least one thrombectomy device, and a package insert comprising instructions for using the mammalian exosomes, tPA and the thrombectomy device in combination to treat or prevent a cerebrovascular injury.


SEQUENCE LISTING (19 sequences)

SEQ ID NO: 1 - DNA, 82 bp, Homo sapiens
gcagtcctctgttagttttgcatagttgcactacaagaagaatgtagttgtgcaaatctatgcaaaactgatggtggcctgc

SEQ ID NO: 2 - DNA, 72 bp, Homo sapiens
tgtcgggtagcttatcagactgatgttgactgttgaatctcatggcaacaccagtcgatgggctgtctgaca

SEQ ID NO: 3 - DNA, 99 bp, Homo sapiens
ccgatgtgtatcctcagctttgagaactgaattccatgggttgtgtcagtgtcagacctctgaaattcagttcttcagctgggatatctctgtcatcgt

SEQ ID NO: 4 - RNA, 22 nt, Homo sapiens
aguuuugcauaguugcacuaca

SEQ ID NO: 5 - RNA, 22 nt, Homo sapiens
uagcuuaucagacugauguuga

SEQ ID NO: 6 - RNA, 22 nt, Homo sapiens
ugagaacugaauuccauggguu

SEQ ID NO: 7 - RNA, 23 nt, Homo sapiens
ugugcaaaucuaugcaaaacuga

SEQ ID NO: 8 - RNA, 82 nt, Homo sapiens
gcaguccucuguuaguuuugcauaguugcacuacaagaagaauguaguugugcaaaucuaugcaaaacugaugguggccugc

SEQ ID NO: 9 - DNA, 82 bp, Homo sapiens; identical to SEQ ID NO: 1

SEQ ID NO: 10 - RNA, 22 nt, Homo sapiens
aguuuugcauaguugcacuaca

SEQ ID NO: 11 - DNA, 22 bp, Homo sapiens
agttttgcatagttgcactaca

SEQ ID NO: 12 - RNA, 72 nt, Homo sapiens
ugucggguagcuuaucagacugauguugacuguugaaucucauggcaacaccagucgaugggcugucugaca

SEQ ID NO: 13 - DNA, 72 bp, Homo sapiens; identical to SEQ ID NO: 2

SEQ ID NO: 14 - RNA, 22 nt, Homo sapiens
uagcuuaucagacugauguuga

SEQ ID NO: 15 - DNA, 22 bp, Homo sapiens
tagcttatcagactgatgttga

SEQ ID NO: 16 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 17 - DNA, 99 bp, Homo sapiens; identical to SEQ ID NO: 3

SEQ ID NO: 18 - RNA, 22 nt, Homo sapiens
ugagaacugaauuccauggguu

SEQ ID NO: 19 - DNA, 22 bp, Homo sapiens
tgagaactgaattccatgggtt